SEQUENCE LISTING (303 sequences)

SEQ ID NO: 1 - protein, 264 aa, synthetic construct
  REGION 1..264: note = >SEQ ID NO: 1 - human CD38-ECD-C-His
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEIHHHHHH

SEQ ID NO: 2 - protein, 264 aa, synthetic construct
  REGION 1..264: note = >SEQ ID NO: 2 - cynomolgus monkey CD38-ECD-C-His
VPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGIHHHHHH

SEQ ID NO: 3 - protein, 157 aa, synthetic construct
  REGION 1..157: note = >SEQ ID NO: 3 - human CD47(C33S)-ECD-Avi-His
QLLFNKTKSVEFTFSNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEGGGGTGGLNDIFEAQKIEWHEGGGHHHHHHHHHH

SEQ ID NO: 4 - protein, 157 aa, synthetic construct
  REGION 1..157: note = >SEQ ID NO: 4 - cynomolgus monkey CD47(C33S)-ECD-Avi-His
QLLFNKTKSVEFTFSNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNEGGGGTGGLNDIFEAQKIEWHEGGGHHHHHHHHHH

SEQ ID NO: 5 - protein, 299 aa, Macaca fascicularis
  REGION 1..299: note = >SEQ ID NO: 5 - Full-length human CD38
ANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 6 - protein, 300 aa, synthetic construct
  REGION 1..300: note = >SEQ ID NO: 6 - Full-length cynomolgus monkey CD38
ANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 7 - protein, 305 aa, Homo sapiens
  REGION 1..305: note = >SEQ ID NO: 7 - Full-length human CD47
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - protein, 305 aa, Macaca fascicularis
  REGION 1..305: note = >SEQ ID NO: 8 - Full-length cynomolgus CD47
QLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 9 - protein, 322 aa, synthetic construct
  REGION 1..322: note = >SEQ ID NO: 9 - human SIRPa-v1-ECD(E31-H352)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAH

SEQ ID NO: 10 - protein, 214 aa, Homo sapiens
  REGION 1..214: note = >SEQ ID NO: 10 - Vk3-15/Jk1 light chain
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 231 aa, synthetic construct
  REGION 1..231: note = >SEQ ID NO: 11 - anti-CD47-UCP01-A1 FAB heavy chain
QVQLVQSGAEVKKPGSSVKVSCKASGGFFRLYAFSWVRQAPGQGLEWMGGIIPTDGFVGYAQKFQGRVTITADESTSTAYMELSSLRSEDTAVYYCARSRYYRPYGEYYVVGYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 226 aa, synthetic construct
  REGION 1..226: note = >SEQ ID NO: 12 - anti-CD47-UCP01-G1 FAB heavy chain
EVQLLESGGGLVQPGGSLRLSCAASGFTFSNYAMAWVRQAPGKGLEWVSVISVEGGLTVYADSVKGRFTISRDNSKNTLYLQMNSLRAEDTAVYYCARGSRDSRGKYGAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 226 aa, synthetic construct
  REGION 1..226: note = >SEQ ID NO: 13 - anti-CD47-UCP01-A2 FAB heavy chain
EVQLLESGGGLVQPGGSLRLSCAASGFTFSRFAMGWVRQAPGKGLEWVSIISPNGHVTAYADSVKGRFTISRDNSKNTLYLQMNSLRAEDTAVYYCARDGLSVSSQGVAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 14 - protein, 225 aa, synthetic construct
  REGION 1..225: note = >SEQ ID NO: 14 - anti-CD47-UCP01-G2 FAB heavy chain
EVQLVESGGGLIQPGGSLRLSCAASGFTVSNSYMTWVRQAPGKGLEWVSVIYKYGKTSYADSVKGRFTISRDNSKNTLYLQMNSLRAEDTAVYYCARGLTVSPYWGGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 15 - protein, 226 aa, synthetic construct
  REGION 1..226: note = >SEQ ID NO: 15 - anti-CD47-UCP01-H2 FAB heavy chain
EVQLVESGGGLIQPGGSLRLSCAASGFTLSRNTMHWVRQAPGKGLEWVSVIYQSGVTRYADSVKGRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 16 - protein, 231 aa, synthetic construct
  REGION 1..231: note = >SEQ ID NO: 16 - anti-CD47-UCP01-A3 FAB heavy chain
EVQLVESGGGLIQPGGSLRLSCAASGFTFTNSWMSWVRQAPGKGLEWVSVIYKYGFTEYADSVKGRFTISRDNSKNTLYLQMNSLRAEDTAVYYCARGSSIAAYAYAAPYRGGWMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 17 - protein, 225 aa, synthetic construct
  REGION 1..225: note = >SEQ ID NO: 17 - anti-CD47-UCP01-B3 FAB heavy chain
EVQLVESGGGLIQPGGSLRLSCAASGFTFSNNYMSWVRQAPGKGLEWVSVIYKKGFTNYADSVKGRFTISRDNSKNTLYLQMNSLRAEDTAVYYCARGLSVDKYDGGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 227 aa, synthetic construct
  REGION 1..227: note = >SEQ ID NO: 18 - anti-CD47-UCP02-C1 FAB heavy chain
QVQLVQSGAEVKKPGSSVKVSCKASGGFFSAHTISWVRQAPGQGLEWMGRITAVRGVPTYAQKFQGRVTITADESTSTAYMELSSLRSEDTAVYYCARASYGGPRYYWDGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 222 aa, synthetic construct
  REGION 1..222: note = >SEQ ID NO: 19 - anti-CD47-UCP02-C2 FAB heavy chain
EVQLVESGGGLIQPGGSLRLSCAASGFSVSSNYMGWVRQAPGKGLEWVSLINKNGQTGYADSVKGRFTISRDNSKNTLYLQMNSLRAEDTAVYYCARSYARSSYQEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 20 - protein, 230 aa, synthetic construct
  REGION 1..230: note = >SEQ ID NO: 20 - anti-CD47-UCP02-B3 FAB heavy chain
QVQLVQSGAEVKKPGSSVKVSCKASGGRFRLYGINWVRQAPGQGLEWMGGISPIDGYAAYAQKFQGRVTITADESTSTAYMELSSLRSEDTAVYYCARSRRPYSASASYPGYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 231 aa, synthetic construct; identical to SEQ ID NO: 11
  REGION 1..231: note = >SEQ ID NO: 21 - anti-CD47-UCP02-H3 FAB heavy chain

SEQ ID NO: 22 - protein, 227 aa, synthetic construct
  REGION 1..227: note = >SEQ ID NO: 22 - anti-CD38-UCP01-B1 FAB heavy chain
QVQLVQSGAEVKKPGSSVKVSCKASGGDFDQYYISWVRQAPGQGLEWMGRIIPTLGDSQYAQKFQGRVTITADESTSTAYMELSSLRSEDTAVYYCARAVIVSPYYYIYVFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 223 aa, synthetic construct
  REGION 1..223: note = >SEQ ID NO: 23 - anti-CD38-UCP01-D1 FAB heavy chain
QVQLVQSGAEVKKPGSSVKVSCKASGGIFNGYLISWVRQAPGQGLEWMGGIIPVNGLADYAQKFQGRVTITADESTSTAYMELSSLRSEDTAVYYCARTIGRYIIAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 24 - protein, 224 aa, synthetic construct
  REGION 1..224: note = >SEQ ID NO: 24 - anti-CD38-UCP01-F1 FAB heavy chain
QVQLVQSGAEVKKPGSSVKVSCKASGGVFSLYTITWVRQAPGQGLEWMGSIIPLLGDPEYAQKFQGRVTITADESTSTAYMELSSLRSEDTAVYYCARYPYPYGSKYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 225 aa, synthetic construct
  REGION 1..225: note = >SEQ ID NO: 25 - anti-CD38-UCP01-G1 FAB heavy chain
QVQLVQSGAEVKKPGSSVKVSCKASGGGFSKYFITWVRQAPGQGLEWMGGVIPKIGYTSYAQKFQGRVTITADESTSTAYMELSSLRSEDTAVYYCARGSDDYASYSPLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 220 aa, synthetic construct
  REGION 1..220: note = >SEQ ID NO: 26 - anti-CD38-UCP01-H1 FAB heavy chain
QVQLVQSGAEVKKPGSSVKVSCKASGDFFSGYAINWVRQAPGQGLEWMGRIIPLLGDTYYAQKFQGRVTITADESTSTAYMELSSLRSEDTAVYYCARSAAGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 219 aa, synthetic construct
  REGION 1..219: note = >SEQ ID NO: 27 - anti-CD38-UCP01-C2 FAB heavy chain
QVQLVQSGAEVKKPGSSVKVSCKASGGYLGIYAISWVRQAPGQGLEWMGRIIPILGEADYAQKFQGRVTITADESTSTAYMELSSLRSEDTAVYYCARGGVWWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 226 aa, synthetic construct
  REGION 1..226: note = >SEQ ID NO: 28 - anti-CD38-UCP01-E2 FAB heavy chain
QVQLVQSGAEVKKPGSSVKVSCKASGGSFSNYAIGWVRQAPGQGLEWMGRIIPVFGSAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 226 aa, synthetic construct
  REGION 1..226: note = >SEQ ID NO: 29 - anti-CD38-UCP02-A7 FAB heavy chain
EVQLLESGGGLVQPGGSLRLSCAASGFTFSFYDMSWVRQAPGKGLEWVSTITLHGGDTYYADSVKGRFTISRDNSKNTLYLQMNSLRAEDTAVYYCARDPSYIYSTSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 30 - protein, 228 aa, synthetic construct
  REGION 1..228: note = >SEQ ID NO: 30 - anti-CD38-UCP02-B1 FAB heavy chain
QVQLVQSGAEVKKPGSSVKVSCKASGGQFSFYAINWVRQAPGQGLEWMGEIIPDYGVTHYAQKFQGRVTITADESTSTAYMELSSLRSEDTAVYYCARWSYTYGIRYYYKLFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 225 aa, synthetic construct
  REGION 1..225: note = >SEQ ID NO: 31 - anti-CD38-UCP02-B7 FAB heavy chain
EVQLVESGGGLVKPGGSLRLSCAASGFSFNEAWMTWVRQAPGKGLEWVGRIKSYSDGGYTGYAAPVKGRFTISRDDSKNTLYLQMNSLKTEDTAVYYCARSPGIPYHYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 32 - protein, 224 aa, synthetic construct
  REGION 1..224: note = >SEQ ID NO: 32 - anti-CD38-UCP02-C3 FAB heavy chain
QVQLVQSGAEVKKPGSSVKVSCKASGDYFNNYAINWVRQAPGQGLEWMGRIIPFLDDANYAQKFQGRVTITADESTSTAYMELSSLRSEDTAVYYCARATSPLYSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 227 aa, synthetic construct; identical to SEQ ID NO: 22
  REGION 1..227: note = >SEQ ID NO: 33 - anti-CD38-UCP02-D1 FAB heavy chain

SEQ ID NO: 34 - protein, 225 aa, synthetic construct
  REGION 1..225: note = >SEQ ID NO: 34 - anti-CD38-UCP02-D5 FAB heavy chain
QVQLVQSGAEVKKPGSSVKVSCKASGGIFSVHAIHWVRQAPGQGLEWMGEIIPVIGEADYAQKFQGRVTITADESTSTAYMELSSLRSEDTAVYYCAREQYYYALGGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 220 aa, synthetic construct
  REGION 1..220: note = >SEQ ID NO: 35 - anti-CD38-UCP02-E5 FAB heavy chain
QVQLVQSGAEVKKPGSSVKVSCKASGGGFRHYFISWVRQAPGQGLEWMGGIIPAAATPYYAQKFQGRVTITADESTSTAYMELSSLRSEDTAVYYCARGYWHWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 224 aa, synthetic construct
  REGION 1..224: note = >SEQ ID NO: 36 - anti-CD38-UCP02-F2 FAB heavy chain
QVQLVQSGAEVKKPGSSVKVSCKASGDVFSLLAIGWVRQAPGQGLEWMGRIIPSLDATHYAQKFQGRVTITADESTSTAYMELSSLRSEDTAVYYCARGRDVLRSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 37 - protein, 224 aa, synthetic construct
  REGION 1..224: note = >SEQ ID NO: 37 - anti-CD38-UCP02-F5 FAB heavy chain
QVQLVQSGAEVKKPGSSVKVSCKASGGVFSYYTISWVRQAPGQGLEWMGRIIPSLGATHYAQKFQGRVTITADESTSTAYMELSSLRSEDTAVYYCARSDSFYYAYYMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 226 aa, synthetic construct
  REGION 1..226: note = >SEQ ID NO: 38 - anti-CD38-UCP02-F6 FAB heavy chain
EVQLLESGGGLVQPGGSLRLSCAASGFTFSFYDMSWVRQAPGKGLEWVSAVSLYGDETYYADSVKGRFTISRDNSKNTLYLQMNSLRAEDTAVYYCAREPVSYDYYGSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 39 - protein, 223 aa, synthetic construct
  REGION 1..223: note = >SEQ ID NO: 39 - anti-CD38-UCP02-H1 FAB heavy chain
QVQLVQSGAEVKKPGSSVKVSCKASGDKFNTYAISWVRQAPGQGLEWMGGITPRYDYAHYAQKFQGRVTITADESTSTAYMELSSLRSEDTAVYYCARSWGFRAGYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 40 - protein, 224 aa, synthetic construct
  REGION 1..224: note = >SEQ ID NO: 40 - anti-CD38-UCP03-B3 FAB heavy chain
QVQLVQSGAEVKKPGSSVKVSCKASGGDFTLYSISWVRQAPGQGLEWMGRIIPTGANANYAQKFQGRVTITADESTSTAYMELSSLRSEDTAVYYCARSWPSLGSGWFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 224 aa, synthetic construct
  REGION 1..224: note = >SEQ ID NO: 41 - anti-CD38-UCP03-C3 FAB heavy chain
QVQLVQSGAEVKKPGSSVKVSCKASGGKFSNYAISWVRQAPGQGLEWMGRIIPQIGDAAYAQKFQGRVTITADESTSTAYMELSSLRSEDTAVYYCARASFLYYSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 42 - protein, 218 aa, synthetic construct
  REGION 1..218: note = >SEQ ID NO: 42 - anti-CD38-UCP03-H5 FAB heavy chain
QVQLVQSGAEVKKPGSSVKVSCKASGGLFSFYAISWVRQAPGQGLEWMGGIIPFFGEPQYAQKFQGRVTITADESTSTAYMELSSLRSEDTAVYYCARGSYRFDVWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 43 - protein, 224 aa, synthetic construct
  REGION 1..224: note = >SEQ ID NO: 43 - anti-CD38-UCP03-B6 FAB heavy chain
QVQLVQSGAEVKKPGSSVKVSCKASGGHISNSAINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 44 - protein, 225 aa, synthetic construct
  REGION 1..225: note = >SEQ ID NO: 44 - anti-CD38-UCP03-E3 FAB heavy chain
QVQLVQSGAEVKKPGSSVKVSCKASGGSFSIYAISWVRQAPGQGLEWMGGIIPNSGDTDYAQKFQGRVTITADESTSTAYMELSSLRSEDTAVYYCARGPYDSYGESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 45 - protein, 224 aa, synthetic construct
  REGION 1..224: note = >SEQ ID NO: 45 - anti-CD38-UCP03-A6 FAB heavy chain
QVQLVQSGAEVKKPGSSVKVSCKASGGDFSLYAISWVRQAPGQGLEWMGGIIPLLGEPVYAQKFQGRVTITADESTSTAYMELSSLRSEDTAVYYCARGHRVYSSSPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 225 aa, synthetic construct; identical to SEQ ID NO: 25
  REGION 1..225: note = >SEQ ID NO: 46 - anti-CD38-UCP03-B1 FAB heavy chain

SEQ ID NO: 47 - protein, 224 aa, synthetic construct
  REGION 1..224: note = >SEQ ID NO: 47 - anti-CD38-UCP03-C2 FAB heavy chain
QVQLVQSGAEVKKPGSSVKVSCKASGGTFSNYAISWVRQAPGQGLEWMGGIIPAFGDAGYAQKFQGRVTITADESTSTAYMELSSLRSEDTAVYYCARGRVLYYSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 223 aa, synthetic construct
  REGION 1..223: note = >SEQ ID NO: 48 - anti-CD38-UCP03-H2 FAB heavy chain
QVQLVQSGAEVKKPGSSVKVSCKASGGEFSIYVISWVRQAPGQGLEWMGRVIPSTGDTNYAQKFQGRVTITADESTSTAYMELSSLRSEDTAVYYCARGKAYYSGWLAIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 49 - protein, 218 aa, synthetic construct
  REGION 1..218: note = >SEQ ID NO: 49 - anti-CD38-UCP03-H3 FAB heavy chain
QVQLVQSGAEVKKPGSSVKVSCKASGGVFSQFPISWVRQAPGQGLEWMGGIIAGYGATEYAQKFQGRVTITADESTSTAYMELSSLRSEDTAVYYCARVYGGIDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 50 - protein, 226 aa, synthetic construct
  REGION 1..226: note = >SEQ ID NO: 50 - anti-CD38-E2-UCP01-A10 FAB heavy chain
QVQLVQSGAEVKKPGSSVKVSCKASGLPDATYAIGWVRQAPGQGLEWMGRIIPVFGSAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 226 aa, synthetic construct
  REGION 1..226: note = >SEQ ID NO: 51 - anti-CD38-E2-UCP02-E7 FAB heavy chain
QVQLVQSGAEVKKPGSSVKVSCKASGGSFSNYAIGWVRQAPGQGLEWMGRIIPALAATHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 52 - protein, 226 aa, synthetic construct
  REGION 1..226: note = >SEQ ID NO: 52 - anti-CD38-E2-UCP02-F3 FAB heavy chain
QVQLVQSGAEVKKPGSSVKVSCKASGGSFSNYAIGWVRQAPGQGLEWMGRIIPVLDAAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 53 - protein, 226 aa, synthetic construct
  REGION 1..226: note = >SEQ ID NO: 53 - anti-CD38-E2-UCP02-D6 FAB heavy chain
QVQLVQSGAEVKKPGSSVKVSCKASGGSFSNYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 54 - protein, 226 aa, synthetic construct
  REGION 1..226: note = >SEQ ID NO: 54 - anti-CD38-E2-UCP02-G8 FAB heavy chain
QVQLVQSGAEVKKPGSSVKVSCKASGGSFSNYAIGWVRQAPGQGLEWMGRIIPALGGVHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 55 - protein, 226 aa, synthetic construct
  REGION 1..226: note = >SEQ ID NO: 55 - anti-CD38-E2-UCP02-F8 FAB heavy chain
QVQLVQSGAEVKKPGSSVKVSCKASGGSFSNYAIGWVRQAPGQGLEWMGRIIPSLDAGHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 56 - protein, 226 aa, synthetic construct
  REGION 1..226: note = >SEQ ID NO: 56 - anti-CD38-E2-UCP02-A5 FAB heavy chain
QVQLVQSGAEVKKPGSSVKVSCKASGGSFSNYAIGWVRQAPGQGLEWMGRIIPDLGAAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 57 - protein, 226 aa, synthetic construct
  REGION 1..226: note = >SEQ ID NO: 57 - anti-CD38-E2-RecA FAB heavy chain
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 58 - protein, 226 aa, synthetic construct
  REGION 1..226: note = >SEQ ID NO: 58 - anti-CD38-E2-RecB FAB heavy chain
QVQLVQSGAEVKKPGSSVKVSCKASGLPDATYAIGWVRQAPGQGLEWMGRIIPALAATHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 59 - protein, 226 aa, synthetic construct
  REGION 1..226: note = >SEQ ID NO: 59 - anti-CD38-E2-RecC FAB heavy chain
QVQLVQSGAEVKKPGSSVKVSCKASGLPDATYAIGWVRQAPGQGLEWMGRIIPVLDAAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - protein, 226 aa, synthetic construct
  REGION 1..226: note = >SEQ ID NO: 60 - anti-CD38-E2-RecD FAB heavy chain
QVQLVQSGAEVKKPGSSVKVSCKASGLPDATYAIGWVRQAPGQGLEWMGRIIPALGGVHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 224 aa, synthetic construct
  REGION 1..224: note = >SEQ ID NO: 61 - anti-CD38-B6-MP01-D9 FAB heavy chain
QVQLVQSGAEVKKPGSSVKVSCKASGGFFSHYIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 62 - protein, 224 aa, synthetic construct
  REGION 1..224: note = >SEQ ID NO: 62 - anti-CD38-B6-MP01-B4 FAB heavy chain
QVQLVQSGAEVKKPGSSVKVSCKASGYEFNTYIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 63 - protein, 224 aa, synthetic construct
  REGION 1..224: note = >SEQ ID NO: 63 - anti-CD38-B6-MP02-C1 FAB heavy chain
QVQLVQSGAEVKKPGSSVKVSCKASGGHQLGKIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 224 aa, synthetic construct
  REGION 1..224: note = >SEQ ID NO: 64 - anti-CD38-B6-MP02-B10 FAB heavy chain
QVQLVQSGAEVKKPGSSVKVSCKASGGHNLGKIINWVRQPPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 65 - protein, 224 aa, synthetic construct
  REGION 1..224: note = >SEQ ID NO: 65 - anti-CD38-B6-MP01-H3a FAB heavy chain
QVQLVQSGAEVKKPGSSVKVSCKASGGPFNANIVNWIRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 66 - protein, 224 aa, synthetic construct
  REGION 1..224: note = >SEQ ID NO: 66 - anti-CD38-B6-MP01-H3b FAB heavy chain
QVQLVQSGAEVKKPGSSVKVSCKASGGPFQSNIVNWIRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 67 - protein, 224 aa, synthetic construct
  REGION 1..224: note = >SEQ ID NO: 67 - anti-CD38-B6-MP01-A4 FAB heavy chain
QVQLVQSGAEVKKPGSSVKVSCKASGGPFSDYVINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 68 - protein, 224 aa, synthetic construct
  REGION 1..224: note = >SEQ ID NO: 68 - anti-CD38-B6-MP01-C2 FAB heavy chain
QVQLVQSGAEVKKPGSSVKVSCKASGGAFSDYVISWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 69 - protein, 224 aa, synthetic construct
  REGION 1..224: note = >SEQ ID NO: 69 - anti-CD38-B6-MP02-C2 FAB heavy chain
QVQLVQSGAEVKKPGSSVKVSCKASGGHGQQRIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 70 - protein, 224 aa, synthetic construct
  REGION 1..224: note = >SEQ ID NO: 70 - anti-CD38-B6-MP02-G11 FAB heavy chain
QVQLVQSGAEVKKPGSSVKVSCKASGGHTLPPIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 71 - protein, 10 aa, synthetic construct
  REGION 1..10: note = >SEQ ID NO: 71 - anti-CD47-UCP01-A1 CDRH1
GGFFRLYAFS

SEQ ID NO: 72 - protein, 10 aa, synthetic construct
  REGION 1..10: note = >SEQ ID NO: 72 - anti-CD47-UCP01-G1 CDRH1
GFTFSNYAMA

SEQ ID NO: 73 - protein, 10 aa, synthetic construct
  REGION 1..10: note = >SEQ ID NO: 73 - anti-CD47-UCP01-A2 CDRH1
GFTFSRFAMG

SEQ ID NO: 74 - protein, 10 aa, synthetic construct
  REGION 1..10: note = >SEQ ID NO: 74 - anti-CD47-UCP01-G2 CDRH1
GFTVSNSYMT

SEQ ID NO: 75 - protein, 10 aa, synthetic construct
  REGION 1..10: note = >SEQ ID NO: 75 - anti-CD47-UCP01-H2 CDRH1
GFTLSRNTMH

SEQ ID NO: 76 - protein, 10 aa, synthetic construct
  REGION 1..10: note = >SEQ ID NO: 76 - anti-CD47-UCP01-A3 CDRH1
GFTFTNSWMS

SEQ ID NO: 77 - protein, 10 aa, synthetic construct
  REGION 1..10: note = >SEQ ID NO: 77 - anti-CD47-UCP01-B3 CDRH1
GFTFSNNYMS

SEQ ID NO: 78 - protein, 10 aa, synthetic construct
  REGION 1..10: note = >SEQ ID NO: 78 - anti-CD47-UCP02-C1 CDRH1
GGFFSAHTIS

SEQ ID NO: 79 - protein, 10 aa, synthetic construct
  REGION 1..10: note = >SEQ ID NO: 79 - anti-CD47-UCP02-C2 CDRH1
GFSVSSNYMG

SEQ ID NO: 80 - protein, 10 aa, synthetic construct
  REGION 1..10: note = >SEQ ID NO: 80 - anti-CD47-UCP02-H3 CDRH1
GGFFRLYAFS

SEQ ID NO: 81 - protein, 10 aa, synthetic construct
  REGION 1..10: note = >SEQ ID NO: 81 - anti-CD47-UCP02-B3 CDRH1
GGRFRLYGIN

SEQ ID NO: 82 - protein, 10 aa, synthetic construct
  REGION 1..10: note = >SEQ ID NO: 82 - anti-CD38-UCP01-B1 CDRH1
GGDFDQYYIS

SEQ ID NO: 83 - protein, 10 aa, synthetic construct
  REGION 1..10: note = >SEQ ID NO: 83 - anti-CD38-UCP01-D1 CDRH1
GGIFNGYLIS

SEQ ID NO: 84 - protein, 10 aa, synthetic construct
  REGION 1..10: note = >SEQ ID NO: 84 - anti-CD38-UCP01-F1 CDRH1
GGVFSLYTIT

SEQ ID NO: 85 - protein, 10 aa, synthetic construct
  REGION 1..10: note = >SEQ ID NO: 85 - anti-CD38-UCP01-G1 CDRH1
GGGFSKYFIT

SEQ ID NO: 86 - protein, 10 aa, synthetic construct
  REGION 1..10: note = >SEQ ID NO: 86 - anti-CD38-UCP01-H1 CDRH1
GDFFSGYAIN

SEQ ID NO: 87 - protein, 10 aa, synthetic construct
  REGION 1..10: note = >SEQ ID NO: 87 - anti-CD38-UCP01-C2 CDRH1
GGYLGIYAIS

SEQ ID NO: 88 - protein, 10 aa, synthetic construct
  REGION 1..10: note = >SEQ ID NO: 88 - anti-CD38-UCP01-E2 CDRH1
GGSFSNYAIG

SEQ ID NO: 89 - protein, 10 aa, synthetic construct
  REGION 1..10: note = >SEQ ID NO: 89 - anti-CD38-UCP02-A7 CDRH1
GFTFSFYDMS

SEQ ID NO: 90 - protein, 10 aa, synthetic construct
  REGION 1..10: note = >SEQ ID NO: 90 - anti-CD38-UCP02-B1 CDRH1
GGQFSFYAIN

SEQ ID NO: 91 - protein, 10 aa, synthetic construct
  REGION 1..10: note = >SEQ ID NO: 91 - anti-CD38-UCP02-B7 CDRH1
GFSFNEAWMT

SEQ ID NO: 92 - protein, 10 aa, synthetic construct
  REGION 1..10: note = >SEQ ID NO: 92 - anti-CD38-UCP02-C3 CDRH1
GDYFNNYAIN

SEQ ID NO: 93 - protein, 10 aa, synthetic construct
  REGION 1..10: note = >SEQ ID NO: 93 - anti-CD38-UCP02-D1 CDRH1
GGDFDQYYIS

SEQ ID NO: 94 - protein, 10 aa, synthetic construct
  REGION 1..10: note = >SEQ ID NO: 94 - anti-CD38-UCP02-D5 CDRH1
GGIFSVHAIH

SEQ ID NO: 95 - protein, 10 aa, synthetic construct
  REGION 1..10: note = >SEQ ID NO: 95 - anti-CD38-UCP02-E5 CDRH1
GGGFRHYFIS

SEQ ID NO: 96 - protein, 10 aa, synthetic construct
  REGION 1..10: note = >SEQ ID NO: 96 - anti-CD38-UCP02-F2 CDRH1
GDVFSLLAIG

SEQ ID NO: 97 - protein, 10 aa, synthetic construct
  REGION 1..10: note = >SEQ ID NO: 97 - anti-CD38-UCP02-F5 CDRH1
GGVFSYYTIS

SEQ ID NO: 98 - protein, 10 aa, synthetic construct
  REGION 1..10: note = >SEQ ID NO: 98 - anti-CD38-UCP02-F6 CDRH1
GFTFSFYDMS

SEQ ID NO: 99 - protein, 10 aa, synthetic construct
  REGION 1..10: note = >SEQ ID NO: 99 - anti-CD38-UCP02-H1 CDRH1
GDKFNTYAIS

SEQ ID NO: 100 - protein, 10 aa, synthetic construct
  REGION 1..10: note = >SEQ ID NO: 100 - anti-CD38-UCP03-B3 CDRH1
GGDFTLYSIS

SEQ ID NO: 101 - protein, 10 aa, synthetic construct
  REGION 1..10: note = >SEQ ID NO: 101 - anti-CD38-UCP03-C3 CDRH1
GGKFSNYAIS

SEQ ID NO: 102 - protein, 10 aa, synthetic construct
  REGION 1..10: note = >SEQ ID NO: 102 - anti-CD38-UCP03-H5 CDRH1
GGLFSFYAIS

SEQ ID NO: 103 - protein, 10 aa, synthetic construct
  REGION 1..10: note = >SEQ ID NO: 103 - anti-CD38-UCP03-B6 CDRH1
GGHISNSAIN

SEQ ID NO: 104 - protein, 10 aa, synthetic construct
  REGION 1..10: note = >SEQ ID NO: 104 - anti-CD38-UCP03-E3 CDRH1
GGSFSIYAIS

SEQ ID NO: 105 - protein, 10 aa, synthetic construct
  REGION 1..10: note = >SEQ ID NO: 105 - anti-CD38-UCP03-A6 CDRH1
GGDFSLYAIS

SEQ ID NO: 106 - protein, 10 aa, synthetic construct
  REGION 1..10: note = >SEQ ID NO: 106 - anti-CD38-UCP03-B1 CDRH1
GGGFSKYFIT

SEQ ID NO: 107 - protein, 10 aa, synthetic construct
  REGION 1..10: note = >SEQ ID NO: 107 - anti-CD38-UCP03-C2 CDRH1
GGTFSNYAIS

SEQ ID NO: 108 - protein, 10 aa, synthetic construct
  REGION 1..10: note = >SEQ ID NO: 108 - anti-CD38-UCP03-H2 CDRH1
GGEFSIYVIS

SEQ ID NO: 109 - protein, 10 aa, synthetic construct
  REGION 1..10: note = >SEQ ID NO: 109 - anti-CD38-UCP03-H3 CDRH1
GGVFSQFPIS

SEQ ID NO: 110 - protein, 10 aa, synthetic construct
  REGION 1..10: note = >SEQ ID NO: 110 - anti-CD38-E2-UCP01-A10 CDRH1
GLPDATYAIG

SEQ ID NO: 111 - protein, 10 aa, synthetic construct
  REGION 1..10: note = >SEQ ID NO: 111 - anti-CD38-E2-UCP02-E7 CDRH1
GGSFSNYAIG

SEQ ID NO: 112 - protein, 10 aa, synthetic construct
  REGION 1..10: note = >SEQ ID NO: 112 - anti-CD38-E2-UCP02-F3 CDRH1
GGSFSNYAIG

SEQ ID NO: 113 - protein, 10 aa, synthetic construct
  REGION 1..10: note = >SEQ ID NO: 113 - anti-CD38-E2-UCP02-D6 CDRH1
GGSFSNYAIG

SEQ ID NO: 114 - protein, 10 aa, synthetic construct
  REGION 1..10: note = >SEQ ID NO: 114 - anti-CD38-E2-UCP02-G8 CDRH1
GGSFSNYAIG

SEQ ID NO: 115 - protein, 10 aa, synthetic construct
  REGION 1..10: note = >SEQ ID NO: 115 - anti-CD38-E2-UCP02-F8 CDRH1
GGSFSNYAIG

SEQ ID NO: 116 - protein, 10 aa, synthetic construct
  REGION 1..10: note = >SEQ ID NO: 116 - anti-CD38-E2-UCP02-A5 CDRH1
GGSFSNYAIG

SEQ ID NO: 117 - protein, 10 aa, synthetic construct
  REGION 1..10: note = >SEQ ID NO: 117 - anti-CD38-E2-RecA CDRH1
GLPDATYAIG

SEQ ID NO: 118 - protein, 10 aa, synthetic construct
  REGION 1..10: note = >SEQ ID NO: 118 - anti-CD38-E2-RecB CDRH1
GLPDATYAIG

SEQ ID NO: 119 - protein, 10 aa, synthetic construct
  REGION 1..10: note = >SEQ ID NO: 119 - anti-CD38-E2-RecC CDRH1
GLPDATYAIG

SEQ ID NO: 120 - protein, 10 aa, synthetic construct
  REGION 1..10: note = >SEQ ID NO: 120 - anti-CD38-E2-RecD CDRH1
GLPDATYAIG

SEQ ID NO: 121 - protein, 10 aa, synthetic construct
  REGION 1..10: note = >SEQ ID NO: 121 - anti-CD38-B6-MP01-D9 CDRH1
GGFFSHYIIN

SEQ ID NO: 122 - protein, 10 aa, synthetic construct
  REGION 1..10: note = >SEQ ID NO: 122 - anti-CD38-B6-MP01-B4 CDRH1
GYEFNTYIIN

SEQ ID NO: 123 - protein, 10 aa, synthetic construct
  REGION 1..10: note = >SEQ ID NO: 123 - anti-CD38-B6-MP02-C1 CDRH1
GGHQLGKIIN

SEQ ID NO: 124 - protein, 10 aa, synthetic construct
  REGION 1..10: note = >SEQ ID NO: 124 - anti-CD38-B6-MP02-B10 CDRH1
GGHNLGKIIN

SEQ ID NO: 125 - protein, 10 aa, synthetic construct
  REGION 1..10: note = >SEQ ID NO: 125 - anti-CD38-B6-MP01-H3a CDRH1
GGPFNANIVN

SEQ ID NO: 126 - protein, 10 aa, synthetic construct
  REGION 1..10: note = >SEQ ID NO: 126 - anti-CD38-B6-MP01-H3b CDRH1
GGPFQSNIVN

SEQ ID NO: 127 - protein, 10 aa, synthetic construct
  REGION 1..10: note = >SEQ ID NO: 127 - anti-CD38-B6-MP01-A4 CDRH1
GGPFSDYVIN

SEQ ID NO: 128 - protein, 10 aa, synthetic construct
  REGION 1..10: note = >SEQ ID NO: 128 - anti-CD38-B6-MP01-C2 CDRH1
GGAFSDYVIS

SEQ ID NO: 129 - protein, 10 aa, synthetic construct
  REGION 1..10: note = >SEQ ID NO: 129 - anti-CD38-B6-MP02-C2 CDRH1
GGHGQQRIIN

SEQ ID NO: 130 - protein, 10 aa, synthetic construct
  REGION 1..10: note = >SEQ ID NO: 130 - anti-CD38-B6-MP02-G11 CDRH1
GGHTLPPIIN

SEQ ID NO: 131 - protein, 10 aa, synthetic construct
  REGION 1..10: note = >SEQ ID NO: 131 - anti-CD47-UCP01-A1 CDRH2
GIIPTDGFVG

SEQ ID NO: 132 - protein, 10 aa, synthetic construct
  REGION 1..10: note = >SEQ ID NO: 132 - anti-CD47-UCP01-G1 CDRH2
VISVEGGLTV

SEQ ID NO: 133 - protein, 10 aa, synthetic construct
  REGION 1..10: note = >SEQ ID NO: 133 - anti-CD47-UCP01-A2 CDRH2
IISPNGHVTA

SEQ ID NO: 134 - protein, 9 aa, synthetic construct
  REGION 1..9: note = >SEQ ID NO: 134 - anti-CD47-UCP01-G2 CDRH2
VIYKYGKTS

SEQ ID NO: 135 - protein, 9 aa, synthetic construct
  REGION 1..9: note = >SEQ ID NO: 135 - anti-CD47-UCP01-H2 CDRH2
VIYQSGVTR

SEQ ID NO: 136 - protein, 9 aa, synthetic construct
  REGION 1..9: note = >SEQ ID NO: 136 - anti-CD47-UCP01-A3 CDRH2
VIYKYGFTE

SEQ ID NO: 137 - protein, 9 aa, synthetic construct
  REGION 1..9: note = >SEQ ID NO: 137 - anti-CD47-UCP01-B3 CDRH2
VIYKKGFTN

SEQ ID NO: 138 - protein, 10 aa, synthetic construct
  REGION 1..10: note = >SEQ ID NO: 138 - anti-CD47-UCP02-C1 CDRH2
RITAVRGVPT

SEQ ID NO: 139 - protein, 10 aa, synthetic construct
  REGION 1..10: note = >SEQ ID NO: 139 - anti-CD47-UCP02-H3 CDRH2
GIIPTDGFVG

SEQ ID NO: 140 - protein, 10 aa, synthetic construct
  REGION 1..10: note = >SEQ ID NO: 140 - anti-CD47-UCP02-B3 CDRH2
GISPIDGYAA

SEQ ID NO: 141 - protein, 9 aa, synthetic construct
  REGION 1..9: note = >SEQ ID NO: 141 - anti-CD47-UCP02-C2 CDRH2
LINKNGQTG

SEQ ID NO: 142 - protein, 10 aa, synthetic construct
  REGION 1..10: note = >SEQ ID NO: 142 - anti-CD38-UCP01-B1 CDRH2
RIIPTLGDSQ

SEQ ID NO: 143 - protein, 10 aa, synthetic construct
  REGION 1..10: note = >SEQ ID NO: 143 - anti-CD38-UCP01-D1 CDRH2
GIIPVNGLAD

SEQ ID NO: 144 - protein, 10 aa, synthetic construct
  REGION 1..10: note = >SEQ ID NO: 144 - anti-CD38-UCP01-F1 CDRH2
SIIPLLGDPE

SEQ ID NO: 145 - protein, 10 aa, synthetic construct
  REGION 1..10: note = >SEQ ID NO: 145 - anti-CD38-UCP01-G1 CDRH2
GVIPKIGYTS

SEQ ID NO: 146 - protein, 10 aa, synthetic construct
  REGION 1..10: note = >SEQ ID NO: 146 - anti-CD38-UCP01-H1 CDRH2
RIIPLLGDTY

SEQ ID NO: 147 - protein, 10 aa, synthetic construct
  REGION 1..10: note = >SEQ ID NO: 147 - anti-CD38-UCP01-C2 CDRH2
RIIPILGEAD

SEQ ID NO: 148 - protein, 10 aa, synthetic construct
  REGION 1..10: note = >SEQ ID NO: 148 - anti-CD38-UCP01-E2 CDRH2
RIIPVFGSAH

SEQ ID NO: 149 - protein, 10 aa, synthetic construct
  REGION 1..10: note = >SEQ ID NO: 149 - anti-CD38-UCP02-A7 CDRH2
TITLHGGDTY

SEQ ID NO: 150 - protein, 10 aa, synthetic construct
  REGION 1..10: note = >SEQ ID NO: 150 - anti-CD38-UCP02-B1 CDRH2
EIIPDYGVTH

SEQ ID NO: 151 - protein, 12 aa, synthetic construct
  REGION 1..12: note = >SEQ ID NO: 151 - anti-CD38-UCP02-B7 CDRH2
RIKSYSDGGYTG

SEQ ID NO: 152 - protein, 10 aa, synthetic construct
  REGION 1..10: note = >SEQ ID NO: 152 - anti-CD38-UCP02-C3 CDRH2
RIIPFLDDAN

SEQ ID NO: 153 - protein, 10 aa, synthetic construct
  REGION 1..10: note = >SEQ ID NO: 153 - anti-CD38-UCP02-D1 CDRH2
RIIPTLGDSQ

SEQ ID NO: 154 - protein, 10 aa, synthetic construct
  REGION 1..10: note = >SEQ ID NO: 154 - anti-CD38-UCP02-D5 CDRH2
EIIPVIGEAD

SEQ ID NO: 155 - protein, 10 aa, synthetic construct
  REGION 1..10: note = >SEQ ID NO: 155 - anti-CD38-UCP02-E5 CDRH2
GIIPAAATPY

SEQ ID NO: 156 - protein, 10 aa, synthetic construct
  REGION 1..10: note = >SEQ ID NO: 156 - anti-CD38-UCP02-F2 CDRH2
RIIPSLDATH

SEQ ID NO: 157 - protein, 10 aa, synthetic construct
  REGION 1..10: note = >SEQ ID NO: 157 - anti-CD38-UCP02-F5 CDRH2
RIIPSLGATH

SEQ ID NO: 158 - protein, 10 aa, synthetic construct
  REGION 1..10: note = >SEQ ID NO: 158 - anti-CD38-UCP02-F6 CDRH2
AVSLYGDETY

SEQ ID NO: 159 - protein, 10 aa, synthetic construct
  REGION 1..10: note = >SEQ ID NO: 159 - anti-CD38-UCP02-H1 CDRH2
GITPRYDYAH

SEQ ID NO: 160 - protein, 10 aa, synthetic construct
  REGION 1..10: note = >SEQ ID NO: 160 - anti-CD38-UCP03-B3 CDRH2
RIIPTGANAN

SEQ ID NO: 161 - protein, 10 aa, synthetic construct
  REGION 1..10: note = >SEQ ID NO: 161 - anti-CD38-UCP03-C3 CDRH2
RIIPQIGDAA

SEQ ID NO: 162 - protein, 10 aa, synthetic construct
  REGION 1..10: note = >SEQ ID NO: 162 - anti-CD38-UCP03-H5 CDRH2
GIIPFFGEPQ

SEQ ID NO: 163 - protein, 10 aa, synthetic construct
  REGION 1..10: note = >SEQ ID NO: 163 - anti-CD38-UCP03-B6 CDRH2
RVIPVIDDAY

SEQ ID NO: 164 - protein, 10 aa, synthetic construct
  REGION 1..10: note = >SEQ ID NO: 164 - anti-CD38-UCP03-E3 CDRH2
GIIPNSGDTD

SEQ ID NO: 165 - protein, 10 aa, synthetic construct
  REGION 1..10: note = >SEQ ID NO: 165 - anti-CD38-UCP03-A6 CDRH2
GIIPLLGEPV

SEQ ID NO: 166 - protein, 10 aa, synthetic construct
  REGION 1..10: note = >SEQ ID NO: 166 - anti-CD38-UCP03-B1 CDRH2
GVIPKIGYTS

SEQ ID NO: 167 - protein, 10 aa, synthetic construct
  REGION 1..10: note = >SEQ ID NO: 167 - anti-CD38-UCP03-C2 CDRH2
GIIPAFGDAG

SEQ ID NO: 168 - protein, 10 aa, synthetic construct
  REGION 1..10: note = >SEQ ID NO: 168 - anti-CD38-UCP03-H2 CDRH2
RVIPSTGDTN

SEQ ID NO: 169 - protein, 10 aa, synthetic construct
  REGION 1..10: note = >SEQ ID NO: 169 - anti-CD38-UCP03-H3 CDRH2
GIIAGYGATE

SEQ ID NO: 170 - protein, 10 aa, synthetic construct
  REGION 1..10: note = >SEQ ID NO: 170 - anti-CD38-E2-UCP01-A10 CDRH2
RIIPVFGSAH

SEQ ID NO: 171 - protein, 10 aa, synthetic construct
  REGION 1..10: note = >SEQ ID NO: 171 - anti-CD38-E2-UCP02-E7 CDRH2
RIIPALAATH

SEQ ID NO: 172 - protein, 10 aa, synthetic construct
  REGION 1..10: note = >SEQ ID NO: 172 - anti-CD38-E2-UCP02-F3 CDRH2
RIIPVLDAAH

SEQ ID NO: 173 - protein, 10 aa, synthetic construct
  REGION 1..10: note = >SEQ ID NO: 173 - anti-CD38-E2-UCP02-D6 CDRH2
RIIPRLDAEH

SEQ ID NO: 174 - protein, 10 aa, synthetic construct
  REGION 1..10: note = >SEQ ID NO: 174 - anti-CD38-E2-UCP02-G8 CDRH2
RIIPALGGVH

SEQ ID NO: 175 - protein, 10 aa, synthetic construct
  REGION 1..10: note = >SEQ ID NO: 175 - anti-CD38-E2-UCP02-A5 CDRH2
RIIPDLGAAH

SEQ ID NO: 176 - protein, 10 aa, synthetic construct
  REGION 1..10: note = >SEQ ID NO: 176 - anti-CD38-E2-UCP02-F8 CDRH2
RIIPSLDAGH

SEQ ID NO: 177 - protein, 10 aa, synthetic construct
  REGION 1..10: note = >SEQ ID NO: 177 - anti-CD38-E2-RecA CDRH2
RIIPRLDAEH

SEQ ID NO: 178 - protein, 10 aa, synthetic construct
  REGION 1..10: note = >SEQ ID NO: 178 - anti-CD38-E2-RecB CDRH2
RIIPALAATH

SEQ ID NO: 179 - protein, 10 aa, synthetic construct
  REGION 1..10: note = >SEQ ID NO: 179 - anti-CD38-E2-RecC CDRH2
RIIPVLDAAH

SEQ ID NO: 180 - protein, 10 aa, synthetic construct
  REGION 1..10: note = >SEQ ID NO: 180 - anti-CD38-E2-RecD CDRH2
RIIPALGGVH

SEQ ID NO: 181 - protein, 10 aa, synthetic construct
  REGION 1..10: note = >SEQ ID NO: 181 - anti-CD38-B6-MP01-D9 CDRH2
RVIPVIDDAY

SEQ ID NO: 182 - protein, 10 aa, synthetic construct
  REGION 1..10: note = >SEQ ID NO: 182 - anti-CD38-B6-MP01-B4 CDRH2
RVIPVIDDAY

SEQ ID NO: 183 - protein, 10 aa, synthetic construct
  REGION 1..10: note = >SEQ ID NO: 183 - anti-CD38-B6-MP02-C1 CDRH2
RVIPVIDDAY

SEQ ID NO: 184 - protein, 10 aa, synthetic construct
  REGION 1..10: note = >SEQ ID NO: 184 - anti-CD38-B6-MP02-B10 CDRH2
RVIPVIDDAY

SEQ ID NO: 185 - protein, 10 aa, synthetic construct
  REGION 1..10: note = >SEQ ID NO: 185 - anti-CD38-B6-MP01-H3a CDRH2
RVIPVIDDAY

SEQ ID NO: 186 - protein, 10 aa, synthetic construct
  REGION 1..10: note = >SEQ ID NO: 186 - anti-CD38-B6-MP01-H3b CDRH2
RVIPVIDDAY

SEQ ID NO: 187 - protein, 10 aa, synthetic construct
  REGION 1..10: note = >SEQ ID NO: 187 - anti-CD38-B6-MP01-A4 CDRH2
RVIPVIDDAY

SEQ ID NO: 188 - protein, 10 aa, synthetic construct
  REGION 1..10: note = >SEQ ID NO: 188 - anti-CD38-B6-MP01-C2 CDRH2
RVIPVIDDAY

SEQ ID NO: 189 - protein, 10 aa, synthetic construct
  REGION 1..10: note = >SEQ ID NO: 189 - anti-CD38-B6-MP02-C2 CDRH2
RVIPVIDDAY

SEQ ID NO: 190 - protein, 10 aa, synthetic construct
  REGION 1..10: note = >SEQ ID NO: 190 - anti-CD38-B6-MP02-G11 CDRH2
RVIPVIDDAY

SEQ ID NO: 191 - protein, 21 aa, synthetic construct
  REGION 1..21: note = >SEQ ID NO: 191 - anti-CD47-UCP01-A1 CDRH3
ARSRYYRPYGEYYVVGYGFAY

SEQ ID NO: 192 - protein, 16 aa, synthetic construct
  REGION 1..16: note = >SEQ ID NO: 192 - anti-CD47-UCP01-G1 CDRH3
ARGSRDSRGKYGAFDV

SEQ ID NO: 193 - protein, 16 aa, synthetic construct
  REGION 1..16: note = >SEQ ID NO: 193 - anti-CD47-UCP01-A2 CDRH3
ARDGLSVSSQGVAFDY

SEQ ID NO: 194 - protein, 16 aa, synthetic construct
  REGION 1..16: note = >SEQ ID NO: 194 - anti-CD47-UCP01-G2 CDRH3
ARGLTVSPYWGGAFDY

SEQ ID NO: 195 - protein, 17 aa, synthetic construct
  REGION 1..17: note = >SEQ ID NO: 195 - anti-CD47-UCP01-H2 CDRH3
ARGRYGVAGGPGYAFDV

SEQ ID NO: 196 - protein, 22 aa, synthetic construct
  REGION 1..22: note = >SEQ ID NO: 196 - anti-CD47-UCP01-A3 CDRH3
ARGSSIAAYAYAAPYRGGWMDV

SEQ ID NO: 197 - protein, 16 aa, synthetic construct
  REGION 1..16: note = >SEQ ID NO: 197 - anti-CD47-UCP01-B3 CDRH3
ARGLSVDKYDGGPFDY

SEQ ID NO: 198 - protein, 17 aa, synthetic construct
  REGION 1..17: note = >SEQ ID NO: 198 - anti-CD47-UCP02-C1 CDRH3
ARASYGGPRYYWDGLDI

SEQ ID NO: 199 - protein, 21 aa, synthetic construct
  REGION 1..21: note = >SEQ ID NO: 199 - anti-CD47-UCP02-H3 CDRH3
ARSRYYRPYGEYYVVGYGFAY

SEQ ID NO: 200 - protein, 20 aa, synthetic construct
  REGION 1..20: note = >SEQ ID NO: 200 - anti-CD47-UCP02-B3 CDRH3
ARSRRPYSASASYPGYGMDV

SEQ ID NO: 201 - protein, 13 aa, synthetic construct
  REGION 1..13: note = >SEQ ID NO: 201 - anti-CD47-UCP02-C2 CDRH3
ARSYARSSYQEDY

SEQ ID NO: 202 - protein, 17 aa, synthetic construct
  REGION 1..17: note = >SEQ ID NO: 202 - anti-CD38-UCP01-B1 CDRH3
ARAVIVSPYYYIYVFDV

SEQ ID NO: 203 - protein, 13 aa, synthetic construct
  REGION 1..13: note = >SEQ ID NO: 203 - anti-CD38-UCP01-D1 CDRH3
ARTIGRYIIAFDY

SEQ ID NO: 204 - protein, 14 aa, synthetic construct
  REGION 1..14: note = >SEQ ID NO: 204 - anti-CD38-UCP01-F1 CDRH3
ARYPYPYGSKYFDI

SEQ ID NO: 205 - protein, 15 aa, synthetic construct
  REGION 1..15: note = >SEQ ID NO: 205 - anti-CD38-UCP01-G1 CDRH3
ARGSDDYASYSPLDS

SEQ ID NO: 206 - protein, 10 aa, synthetic construct
  REGION 1..10: note = >SEQ ID NO: 206 - anti-CD38-UCP01-H1 CDRH3
ARSAAGWFAY

SEQ ID NO: 207 - protein, 9 aa, synthetic construct
  REGION 1..9: note = >SEQ ID NO: 207 - anti-CD38-UCP01-C2 CDRH3
ARGGVWWDY

SEQ ID NO: 208 - protein, 16 aa, synthetic construct
  REGION 1..16: note = >SEQ ID NO: 208 - anti-CD38-UCP01-E2 CDRH3
ARGLGYYLYSSYYFDI

SEQ ID NO: 209 - protein, 16 aa, synthetic construct
  REGION 1..16: note = >SEQ ID NO: 209 - anti-CD38-UCP02-A7 CDRH3
ARDPSYIYSTSGYFDY

SEQ ID NO: 210 - protein, 18 aa, synthetic construct
  REGION 1..18: note = >SEQ ID NO: 210 - anti-CD38-UCP02-B1 CDRH3
ARWSYTYGIRYYYKLFDP

SEQ ID NO: 211 - protein, 13 aa, synthetic construct
  REGION 1..13: note = >SEQ ID NO: 211 - anti-CD38-UCP02-B7 CDRH3
ARSPGIPYHYFDV

SEQ ID NO: 212 - protein, 14 aa, synthetic construct
  REGION 1..14: note = >SEQ ID NO: 212 - anti-CD38-UCP02-C3 CDRH3
ARATSPLYSYYFDY

SEQ ID NO: 213 - protein, 17 aa, synthetic construct
  REGION 1..17: note = >SEQ ID NO: 213 - anti-CD38-UCP02-D1 CDRH3
ARAVIVSPYYYIYVFDV

SEQ ID NO: 214 - protein, 15 aa, synthetic construct
  REGION 1..15: note = >SEQ ID NO: 214 - anti-CD38-UCP02-D5 CDRH3
AREQYYYALGGGFDY

SEQ ID NO: 215 - protein, 10 aa, synthetic construct
  REGION 1..10: note = >SEQ ID NO: 215 - anti-CD38-UCP02-E5 CDRH3
ARGYWHWFDY

SEQ ID NO: 216 - protein, 14 aa, synthetic construct
  REGION 1..14: note = >SEQ ID NO: 216 - anti-CD38-UCP02-F2 CDRH3
ARGRDVLRSYYFDI

SEQ ID NO: 217 - protein, 14 aa, synthetic construct
  REGION 1..14: note = >SEQ ID NO: 217 - anti-CD38-UCP02-F5 CDRH3
ARSDSFYYAYYMDL

SEQ ID NO: 218 - protein, 16 aa, synthetic construct
  REGION 1..16: note = >SEQ ID NO: 218 - anti-CD38-UCP02-F6 CDRH3
AREPVSYDYYGSYFDY

SEQ ID NO: 219 - protein, 13 aa, synthetic construct
  REGION 1..13: note = >SEQ ID NO: 219 - anti-CD38-UCP02-H1 CDRH3
ARSWGFRAGYLDY

SEQ ID NO: 220 - protein, 14 aa, synthetic construct
  REGION 1..14: note = >SEQ ID NO: 220 - anti-CD38-UCP03-B3 CDRH3
ARSWPSLGSGWFDI

SEQ ID NO: 221 - protein, 14 aa, synthetic construct
  REGION 1..14: note = >SEQ ID NO: 221 - anti-CD38-UCP03-C3 CDRH3
ARASFLYYSYYFDY

SEQ ID NO: 222 - protein, 9 aa, synthetic construct
  REGION 1..9: note = >SEQ ID NO: 222 - anti-CD38-UCP03-H5 CDRH3
ARGSYRFDV

SEQ ID NO: 223 - protein, 14 aa, synthetic construct
  REGION 1..14: note = >SEQ ID NO: 223 - anti-CD38-UCP03-B6 CDRH3
ARSRGYYGSFYFDI

SEQ ID NO: 224 - protein, 15 aa, synthetic construct
  REGION 1..15: note = >SEQ ID NO: 224 - anti-CD38-UCP03-E3 CDRH3
ARGPYDSYGESYFDY

SEQ ID NO: 225 - protein, 14 aa, synthetic construct
  REGION 1..14: note = >SEQ ID NO: 225 - anti-CD38-UCP03-A6 CDRH3
ARGHRVYSSSPFDY

SEQ ID NO: 226 - protein, 15 aa, synthetic construct
  REGION 1..15: note = >SEQ ID NO: 226 - anti-CD38-UCP03-B1 CDRH3
ARGSDDYASYSPLDS

SEQ ID NO: 227 - protein, 14 aa, synthetic construct
  REGION 1..14: note = >SEQ ID NO: 227 - anti-CD38-UCP03-C2 CDRH3
ARGRVLYYSYYFDY

SEQ ID NO: 228 - protein, 13 aa, synthetic construct
  REGION 1..13: note = >SEQ ID NO: 228 - anti-CD38-UCP03-H2 CDRH3
ARGKAYYSGWLAI

SEQ ID NO: 229 - protein, 8 aa, synthetic construct
  REGION 1..8: note = >SEQ ID NO: 229 - anti-CD38-UCP03-H3 CDRH3
ARVYGGID

SEQ ID NO: 230 - protein, 16 aa, synthetic construct
  REGION 1..16: note = >SEQ ID NO: 230 - anti-CD38-E2-UCP01-A10 CDRH3
ARGLGYYLYSSYYFDI

SEQ ID NO: 231 - protein, 16 aa, synthetic construct
  REGION 1..16: note = >SEQ ID NO: 231 - anti-CD38-E2-UCP02-E7 CDRH3
ARGLGYYLYSSYYFDI

SEQ ID NO: 232 - protein, 16 aa, synthetic construct
  REGION 1..16: note = >SEQ ID NO: 232 - anti-CD38-E2-UCP02-F3 CDRH3
ARGLGYYLYSSYYFDI

SEQ ID NO: 233 - protein, 16 aa, synthetic construct
  REGION 1..16: note = >SEQ ID NO: 233 - anti-CD38-E2-UCP02-D6 CDRH3
ARGLGYYLYSSYYFDI

SEQ ID NO: 234 - protein, 16 aa, synthetic construct
  REGION 1..16: note = >SEQ ID NO: 234 - anti-CD38-E2-UCP02-G8 CDRH3
ARGLGYYLYSSYYFDI

SEQ ID NO: 235 - protein, 16 aa, synthetic construct
  REGION 1..16: note = >SEQ ID NO: 235 - anti-CD38-E2-UCP02-F8 CDRH3
ARGLGYYLYSSYYFDI

SEQ ID NO: 236 - protein, 16 aa, synthetic construct
  REGION 1..16: note = >SEQ ID NO: 236 - anti-CD38-E2-UCP02-A5 CDRH3
ARGLGYYLYSSYYFDI

SEQ ID NO: 237 - protein, 16 aa, synthetic construct
  REGION 1..16: note = >SEQ ID NO: 237 - anti-CD38-E2-RecA CDRH3
ARGLGYYLYSSYYFDI

SEQ ID NO: 238 - protein, 16 aa, synthetic construct
  REGION 1..16: note = >SEQ ID NO: 238 - anti-CD38-E2-RecB CDRH3
ARGLGYYLYSSYYFDI

SEQ ID NO: 239 - protein, 16 aa, synthetic construct
  REGION 1..16: note = >SEQ ID NO: 239 - anti-CD38-E2-RecC CDRH3
ARGLGYYLYSSYYFDI

SEQ ID NO: 240 - protein, 16 aa, synthetic construct
  REGION 1..16: note = >SEQ ID NO: 240 - anti-CD38-E2-RecD CDRH3
ARGLGYYLYSSYYFDI

SEQ ID NO: 241 - protein, 14 aa, synthetic construct
  REGION 1..14: note = >SEQ ID NO: 241 - anti-CD38-B6-MP01-D9 CDRH3
ARSRGYYGSFYFDI

SEQ ID NO: 242 - protein, 14 aa, synthetic construct
  REGION 1..14: note = >SEQ ID NO: 242 - anti-CD38-B6-MP01-B4 CDRH3
ARSRGYYGSFYFDI

SEQ ID NO: 243 - protein, 14 aa, synthetic construct
  REGION 1..14: note = >SEQ ID NO: 243 - anti-CD38-B6-MP02-C1 CDRH3
ARSRGYYGSFYFDI

SEQ ID NO: 244 - protein, 14 aa, synthetic construct
  REGION 1..14: note = >SEQ ID NO: 244 - anti-CD38-B6-MP02-B10 CDRH3
ARSRGYYGSFYFDI

SEQ ID NO: 245 - protein, 14 aa, synthetic construct
  REGION 1..14: note = >SEQ ID NO: 245 - anti-CD38-B6-MP01-H3a CDRH3
ARSRGYYGSFYFDI

SEQ ID NO: 246 - protein, 14 aa, synthetic construct
  REGION 1..14: note = >SEQ ID NO: 246 - anti-CD38-B6-MP01-H3b CDRH3
ARSRGYYGSFYFDI

SEQ ID NO: 247 - protein, 14 aa, synthetic construct
  REGION 1..14: note = >SEQ ID NO: 247 - anti-CD38-B6-MP01-A4 CDRH3
ARSRGYYGSFYFDI

SEQ ID NO: 248 - protein, 14 aa, synthetic construct
  REGION 1..14: note = >SEQ ID NO: 248 - anti-CD38-B6-MP01-C2 CDRH3
ARSRGYYGSFYFDI

SEQ ID NO: 249 - protein, 14 aa, synthetic construct
  REGION 1..14: note = >SEQ ID NO: 249 - anti-CD38-B6-MP02-C2 CDRH3
ARSRGYYGSFYFDI

SEQ ID NO: 250 - protein, 14 aa, synthetic construct
  REGION 1..14: note = >SEQ ID NO: 250 - anti-CD38-B6-MP02-G11 CDRH3
ARSRGYYGSFYFDI

SEQ ID NO: 251 - protein, 10 aa, synthetic construct
  REGION 1..10: note = >SEQ ID NO: 251 - anti-CD38-E2 derivatives CDRH1 consensus
GLPDATYAIQ

SEQ ID NO: 252 - protein, 10 aa, synthetic construct
  REGION 1..10: note = >SEQ ID NO: 252 - anti-CD38-E2 derivatives CDRH2 consensus
  VARIANT 5: note = Where X at position 5 can be V, A, S, D, R; Where X at position 7 can be A, G, D; Where X at position 2 can be G or L; Where X at position 8 can be A, G; Where X at position 9 can be V, A, G, T, E.
RIIPXFXXXH

SEQ ID NO: 253 - protein, 16 aa, synthetic construct
  REGION 1..16: note = >SEQ ID NO: 253 - anti-CD38-E2 derivatives CDRH3 consensus
ARGLGYYLYSSYYFDI

SEQ ID NO: 254 - skipped

SEQ ID NO: 255 - protein, 10 aa, synthetic construct
  REGION 1..10: note = >SEQ ID NO: 255 - anti-CD38-B6 derivatives CDRH2 consensus
RVIPVIDDAY

SEQ ID NO: 256 - protein, 14 aa, synthetic construct
  REGION 1..14: note = >SEQ ID NO: 256 - anti-CD38-B6 derivatives CDRH3 consensus
ARSRGYYGSFYFDI

SEQ ID NO: 257 - protein, 226 aa, synthetic construct
  REGION 1..226: note = >SEQ ID NO: 257 - BEAT (A) Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 258 - protein, 226 aa, synthetic construct
  REGION 1..226: note = >SEQ ID NO: 258 - BEAT (B) Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 259 - protein, 452 aa, synthetic construct
  REGION 1..452: note = >SEQ ID NO: 259 - BEAT CD38/CD47-3, -6, -19, -38 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 260 - protein, 452 aa, synthetic construct
  REGION 1..452: note = >SEQ ID NO: 260 - BEAT CD38/CD47-3 BEAT (B) Hc
QVQLVQSGAEVKKPGSSVKVSCKASGGSFSNYAIGWVRQAPGQGLEWMGRIIPVLDAAHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 261 - protein, 452 aa, synthetic construct
  REGION 1..452: note = >SEQ ID NO: 261 BEAT CD38/CD47-6 BEAT (B) Hc
QVQLVQSGAEVKKPGSSVKVSCKASGGSFSNYAIGWVRQAPGQGLEWMGRIIPSLDAGHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 262 - protein, 452 aa, synthetic construct
  REGION 1..452: note = >SEQ ID NO: 262 - BEAT CD38/CD47-19 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 263 - protein, 450 aa, synthetic construct
  REGION 1..450: note = >SEQ ID NO: 263 - Trastuzumab Hc
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 214 aa, synthetic construct
  REGION 1..214: note = >SEQ ID NO: 264 - Trastuzumab Lc
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 214 aa, synthetic construct
  REGION 1..214: note = >SEQ ID NO: 265 - ABC IgG1 Lc
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 446 aa, synthetic construct
  REGION 1..446: note = >SEQ ID NO: 266 - ABC IgG1 Hc
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 267 - protein, 444 aa, synthetic construct
  REGION 1..444: note = >SEQ ID NO: 267 - 5F9-G4 Hc
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 268 - protein, 219 aa, synthetic construct
  REGION 1..219: note = >SEQ ID NO: 268 - 5F9-G4 Lc
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 308 aa, synthetic construct
  REGION 1..308: note = >SEQ ID NO: 269 - human FcRn-Avi-His
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSGGGGTGGLNDIFEAQKIEWHEGGGHHHHHHHHHH

SEQ ID NO: 270 - protein, 99 aa, synthetic construct [sequence omitted]

SEQ ID NO: 271 - protein, 185 aa, synthetic construct
  REGION 1..185: note = >SEQ ID NO: 271 - human FcgR2a-C-10His
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPHHHHHHHHHH

SEQ ID NO: 272 - protein, 178 aa, synthetic construct
  REGION 1..178: note = >SEQ ID NO: 272 - human FcgR2b-C-His
APPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPHHHHHH

SEQ ID NO: 273 - protein, 185 aa, synthetic construct
  REGION 1..185: note = >SEQ ID NO: 273 - human FcgR3a-C-His
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGSAHHHHHH

SEQ ID NO: 274 - protein, 452 aa, synthetic construct
  REGION 1..452: note = >SEQ ID NO: 274 - BEAT CD38/CD47-21 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEETISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 275 - protein, 452 aa, synthetic construct
  REGION 1..452: note = >SEQ ID NO: 275 - BEAT CD38/CD47-21 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEETISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 276 - protein, 452 aa, synthetic construct
  REGION 1..452: note = >SEQ ID NO: 276 - BEAT CD38/CD47-22 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 277 - protein, 452 aa, synthetic construct
  REGION 1..452: note = >SEQ ID NO: 277 - BEAT CD38/CD47-22, -42 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 278 - protein, 452 aa, synthetic construct
  REGION 1..452: note = >SEQ ID NO: 278 - BEAT CD38/CD47-24, -34, -40 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIEATISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 279 - protein, 452 aa, synthetic construct
  REGION 1..452: note = >SEQ ID NO: 279 - BEAT CD38/CD47-24 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIEATISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 280 - protein, 452 aa, synthetic construct
  REGION 1..452: note = >SEQ ID NO: 280 - BEAT CD38/CD47-42, -48, -60 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVNNKALPAPEEKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 281 - protein, 452 aa, synthetic construct
  REGION 1..452: note = >SEQ ID NO: 281 - BEAT CD38/CD47-23, -32, -39 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIAKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 282 - protein, 452 aa, synthetic construct
  REGION 1..452: note = >SEQ ID NO: 282 - BEAT CD38/CD47-23 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIAKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 283 - protein, 452 aa, synthetic construct
  REGION 1..452: note = >SEQ ID NO: 283 - BEAT CD38/CD47-25 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVNNKALPAPIAKTISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 284 - protein, 452 aa, synthetic construct
  REGION 1..452: note = >SEQ ID NO: 284 - BEAT CD38/CD47-25 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVNNKALPAPIAKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 285 - protein, 452 aa, synthetic construct
  REGION 1..452: note = >SEQ ID NO: 285 - BEAT CD38/CD47-26 BEAT (A) Hc
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVNNKALPAPIEATISKAKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPG

SEQ ID NO: 286 - protein, 452 aa, synthetic construct
  REGION 1..452: note = >SEQ ID NO: 286 - BEAT CD38/CD47-26 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVNNKALPAPIEATISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 287 - protein, 693 aa, synthetic construct
  REGION 1..693: note = >SEQ ID NO: 287 - BEAT CD38/CD47-32 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIAKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 288 - protein, 693 aa, synthetic construct
  REGION 1..693: note = >SEQ ID NO: 288 - BEAT CD38/CD47-34 BEAT (B) Hc
QVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIEATISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 289 - protein, 691 aa, synthetic construct
  REGION 1..691: note = >SEQ ID NO: 289 - BEAT CD38/CD47-38 BEAT (B) Hc
QVQLVQSGAEVKKPGSSVKVSCKASGGHISNSAINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 290 - protein, 691 aa, synthetic construct
  REGION 1..691: note = >SEQ ID NO: 290 - BEAT CD38/CD47-39 BEAT (B) Hc
QVQLVQSGAEVKKPGSSVKVSCKASGGHISNSAINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIAKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 291 - protein, 691 aa, synthetic construct
  REGION 1..691: note = >SEQ ID NO: 291 - BEAT CD38/CD47-40 BEAT (B) Hc
QVQLVQSGAEVKKPGSSVKVSCKASGGHISNSAINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHDDPEVKFNWYVDGVEVHNAKTKPREEQYNATYRVVSVLTVLHQDWLNGKEYKCKVNNKALPAPIEATISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 292 - protein, 691 aa, synthetic construct
  REGION 1..691: note = >SEQ ID NO: 292 - BEAT CD38/CD47-48 BEAT (B) Hc
QVQLVQSGAEVKKPGSSVKVSCKASGGHISNSAINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 293 - protein, 691 aa, synthetic construct
  REGION 1..691: note = >SEQ ID NO: 293 - BEAT CD38/CD47-60 BEAT (B) Hc
QVQLVQSGAEVKKPGSSVKVSCKASGGFFSHYIINWVRQAPGQGLEWMGRVIPVIDDAYYAQKFQGRVTITADESTSTAYMELSSLRSEDTAVYYCARSRGYYGSFYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGSSVKVSCKASGLPDATYAIGWVRQAPGQGLEWMGRIIPRLDAEHYAQKFQGRVTITADESTSTAYMELSSLRSEDTAVYYCARGLGYYLYSSYYFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVNNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPG

SEQ ID NO: 294 - protein, 122 aa, Homo sapiens
  REGION 1..122: note = >SEQ ID NO: 294 - human CD38 isoform P28907-2
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKK

SEQ ID NO: 295 - protein, 300 aa, Homo sapiens
  REGION 1..300: note = >SEQ ID NO: 295 - human CD38 isoform P28907-1
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 296 - protein, 300 aa, Homo sapiens; identical to SEQ ID NO: 295
  REGION 1..300: note = >SEQ ID NO: 296- human CD38 isoform P28907-E

SEQ ID NO: 297 - protein, 4 aa, synthetic construct
  REGION 1..4: note = >SEQ ID NO: 297- 4G T linker
GGGT

SEQ ID NO: 298 - protein, 15 aa, synthetic construct
  REGION 1..15: note = >SEQ ID NO: 298- 4GS linker
GGGGSGGGGSGGGGS

SEQ ID NO: 299 - protein, 226 aa, synthetic construct
  REGION 1..226: note = FAB Anti-CD47-H2 with G65S mutation
EVQLVESGGGLIQPGGSLRLSCAASGFTLSRNTMHWVRQAPGKGLEWVSVIYQSGVTRYADSVKSRFTISRDNSKNTLYLQMNSLRAEDTAVYYCARGRYGVAGGPGYAFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 300 - protein, 323 aa, synthetic construct
  REGION 1..323: note = Human CD47 Isoform OA3-323
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 301 - protein, 292 aa, synthetic construct
  REGION 1..292: note = Human CD47 Isoform OA3-293
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 302 - protein, 305 aa, synthetic construct
  REGION 1..305: note = Human CD47Isoform OA3-305
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 303 - protein, 311 aa, synthetic construct
  REGION 1..311: note = Human CD47 Isoform OA3-312
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

(19) United States
(12) Patent Application Publication
(10) Pub. No.: US 2026/0365750 A1
(43) Pub. Date: Nov. 19, 2026

(54) CD47/CD38 BISPECIFIC ANTIBODIES AND METHODS OF USE TO TREAT LEUKEMIA

(71) Applicant: IGI Therapeutics SA, La Chaux-de-Fonds (CH)

(72) Inventors: Stefano SAMMICHELI, La Chaux-de-Fonds (CH); Mario PERRO, La Chaux-de-Fonds (CH); Camille GRANDCLEMENT, La Chaux-de-Fonds (CH)

(21) Appl. No.: 19/135,726

(22) PCT Filed: Dec. 7, 2023
(86) PCT No.: PCT/EP2023/084717

§ 371 Date: Jun. 4, 2025

(30) Foreign Application Priority Data
Dec. 7, 2022 (EP) 22211979.4

(51) Int. Cl. C07K 16/28; A61P 35/02

(52) U.S. Cl. CPC C07K 16/2896; A61P 35/02; C07K 16/2803; C07K 2317/31

(57) ABSTRACT

The present invention relates to a bispecific antibody comprising at least one binding portion which binds to human CD47 and at least two binding portions which bind to human CD38, for use in the treatment of Acute myeloid leukemia or T-cell acute lymphoblastic leukemia.

### FIELD OF THE INVENTION

The present invention relates a bispecific antibody comprising at least one binding portion which binds to human CD47 and at least two binding portions which bind to human CD38, for use in the treatment of Acute myeloid leukemia or T-cell acute lymphoblastic leukemia.

### BACKGROUND OF THE INVENTION

CD47 is a surface protein expressed ubiquitously and is the ligand for the signal regulatory protein alpha (SIRPα), which is constitutively expressed on myeloid cells. The binding of CD47 to SIRPa triggers a signal transduction cascade that leads to the inhibition of phagocytosis, also called a ‘don't eat me’ signal. CD47 is overexpressed in many cancers and its increased expression has been associated to increased tumor invasion and metastasis, tumor progression and shortened patient survival. The expression of CD47 on the surface of tumor cells allows them to overcome intrinsic prophagocytic signals, and thereby escape phagocytosis. Blocking of the ‘don't eat me’ signal with an antibody has demonstrated efficacy both preclinically and clinically across various malignancies including acute myeloid leukemia (AML), diffuse large B cell lymphoma (DLBCL), as well as in combination with PD(L)1 checkpoint blockade inhibitor in solid tumors. CD47 is also highly expressed on newly-formed red blood cells (RBCs) to prevent their macrophage-dependent recycling over senescent RBCs that express lower level of CD47. Therefore, antibodies targeting CD47 should be carefully designed to avoid sink, due to ubiquitous CD47 expression, and to prevent hemolytic anemia due to the on target off tumor on RBCs. In AML, unleashing tumor cell phagocytosis using anti-CD47 mAb (magrolimab) shows clinical benefit in combination with demethylating agents and BCL-2 inhibitors.

CD38 is a surface protein that is expressed on plasma cells and is highly expressed in hematological malignancies including MM, AML, T-cell acute lymphoblastic leukemia (T-ALL) and DLBCL. The expression of CD38 in hematologic malignancies led to the development of two CD38 mAb therapies, daratumumab and isatuximab, both approved for the treatment of MM. These therapies have significantly prolonged survival of patients, especially when used in combination therapies with proteasome inhibitors (PI) and immunomodulatory drugs (IMIDs).

Despite significant breakthroughs in the treatment of hematological malignancies, most patients eventually relapse all treatment options. The treatment of CD38-overexpressing cancers such as acute myeloid leukemia, multiple myeloma and T-cell acute lymphoblastic leukemia still remains an unmet need.

### DETAILED DESCRIPTION

Many human cancers express high levels of CD47, a surface protein expressed ubiquitously which is the ligand for the signal regulatory protein alpha (SIRPα), which is constitutively expressed on myeloid cells. The binding of CD47 to SIRPα triggers a signal transduction cascade that leads to the inhibition of phagocytosis, also called a ‘don't eat me’ signal, CD47 overexpression has been therefore associated to increased tumor invasion and metastasis, tumor progression and shortened patient survival. CD38 is a surface protein expressed on plasma cells and is highly expressed in Multiple Myeloma (MM), the second most common hematological malignancy worldwide, as well as in other hematological malignancies, such as Acute myeloid leukemia (AML) and T-cell acute lymphoblastic leukemia (T-ALL).

To block the CD47-SIRPα interaction selectively in CD38 expressing cancer cells, the inventors have set out to provide improved effector cell redirecting antibody-based therapeutics, that specifically act on the tumor cells.

The present invention relates to a bispecific antibody comprising at least two binding portions, at least one of which binds to human CD38 and at least one of which binds to human CD47.

The present invention also relates to a bispecific antibody comprising at least one binding portion which binds to human CD47 and at least two binding portions which bind to human CD38.

The present invention also relates to a bispecific antibody comprising at least one binding portion which binds to human CD47 and at least two binding portions which bind to human CD38, wherein the at least two CD38 binding portions are monoparatopic.

The present invention also relates to a bispecific antibody comprising at least one binding portion which binds to human CD47 and at least two binding portions which bind to human CD38, wherein the at least two CD38 binding portions are biparatopic.

The present invention also relates to a bispecific antibody comprising at least two binding portions, at least one of which binds to human CD38 and at least one of which binds to human CD47, wherein at least one of the binding portions which binds to human CD47 can also bind to cynomologus CD47.

The present invention also relates to a bispecific antibody comprising at least two binding portions, at least one of which binds to human CD38 and at least one of which binds to human CD47, wherein at least one of the binding portions which binds to human CD38 can also bind to cynomologus CD38.

The present invention also relates to the bispecific antibody above for use as a medicament.

More in particular, for use in treating multiple myeloma, acute lymphoblastic leukemia, chronic lymphocytic leukemia, acute myeloid leukemia, lymphoma, breast cancer such as Her2+ breast cancer, prostate cancer, cervival cancer, germinal center B-cell lympohoma or B-cell acute lymphoblastic leukemia, Chronic lymphocytic leukemia (CLL), Myelodisplastic syndrome (MDS), Non-Hodgkin lymphoma, diffuse large B-cell lymphoma, non small cell lung cancer (NSCLC), Hepatocellular carcinoma (HCC), High-grade serous ovarian carcinoma, peritoneal cancer.

In particular the present invention relates to a bispecific antibody comprising at least one binding portion which binds to human CD47 and at least two binding portions which bind to human CD38, for use in the treatment of a subject having Acute myeloid leukemia (AML) or T-cell acute lymphoblastic leukemia (T-ALL); or for use in preventing Acute myeloid leukemia (AML) or T-cell acute lymphoblastic leukemia (T-ALL) in a subject.

The present invention also relates to a method for treating a subject having Acute myeloid leukemia or T-cell acute lymphoblastic leukemia or for preventing the development of Acute myeloid leukemia or T-cell acute lymphoblastic leukemia in a subject comprising administering a therapeutically effective amount of the bispecific antibody disclosed herein.

The present invention also relates to use of a bispecific antibody comprising at least one binding portion which binds to human CD47 and at least two binding portions which bind to human CD38 as medicament in the treatment of a subject having Acute myeloid leukemia or T-cell acute lymphoblastic leukemia or for the prevention of the development of Acute myeloid leukemia or T-cell acute lymphoblastic leukemia in a subject.

Specifically, the bispecific antibody for use in the treatment of Acute myeloid leukemia or T-cell acute lymphoblastic leukemia, has at least two CD38 binding portions which are biparatopic.

In certain embodiments the bispecific antibody for use in the treatment of Acute myeloid leukemia or T-cell acute lymphoblastic leukemia has at least one binding portion which binds to human CD38 comprises a CDR set selected from the group comprising: SEQ ID NO: 103, SEQ ID NO: 163, SEQ ID NO: 223; SEQ ID NO: 117, SEQ ID NO: 177, SEQ ID NO: 237; SEQ ID NO: 121; SEQ ID NO: 181, SEQ ID NO: 241.

In certain embodiments the bispecific antibody for use in the treatment of Acute myeloid leukemia or T-cell acute lymphoblastic leukemia has said at least one binding portion which binds to human CD38 comprises a CDR set comprising the amino acid sequence of SEQ ID NO: 117, SEQ ID NO: 177, SEQ ID NO: 237; and a CDR set comprising the amino acid sequence of SEQ ID NO: 121; SEQ ID NO: 181, SEQ ID NO: 241.

In certain embodiments the bispecific antibody for use in the treatment of Acute myeloid leukemia or T-cell acute lymphoblastic leukemia has at least one binding portion which binds to human CD47 comprises a CDR set comprising the amino acid sequence of SEQ ID NO: 75, SEQ ID NO: 135, SEQ ID NO: 195.

The present invention also relates to a method of treatment Acute myeloid leukemia (AML) or T-cell acute lymphoblastic leukemia (T-ALL) using a bispecific antibody comprising at least one binding portion which binds to human CD47 and at least two binding portions which bind to human CD38.

Specifically, the present invention relates to a method of treatment Acute myeloid leukemia (AML) or T-cell acute lymphoblastic leukemia (T-ALL) using a bispecific antibody that has at least two CD38 binding portions which are biparatopic.

Specifically, the present invention relates to a method of treatment Acute myeloid leukemia (AML) or T-cell acute lymphoblastic leukemia (T-ALL) using a bispecific antibody that has at least one binding portion which binds to human CD38 comprises a CDR set selected from the group comprising: SEQ ID NO: 103, SEQ ID NO: 163, SEQ ID NO: 223; SEQ ID NO: 117, SEQ ID NO: 177, SEQ ID NO: 237; SEQ ID NO: 121; SEQ ID NO: 181, SEQ ID NO: 241.

Specifically, the present invention relates to a method of treatment Acute myeloid leukemia (AML) or T-cell acute lymphoblastic leukemia (T-ALL) using a bispecific antibody that has at least one binding portion which binds to human CD38 comprises a CDR set comprising the amino acid sequence of SEQ ID NO: 117, SEQ ID NO: 177, SEQ ID NO: 237; and a CDR set comprising the amino acid sequence of SEQ ID NO: 121; SEQ ID NO: 181, SEQ ID NO: 241.

Specifically, the present invention relates to a method of treatment Acute myeloid leukemia (AML) or T-cell acute lymphoblastic leukemia (T-ALL) using a bispecific antibody that has at least one binding portion which binds to human CD47 comprises a CDR set comprising the amino acid sequence of SEQ ID NO: 75, SEQ ID NO: 135, SEQ ID NO: 195.

The present invention also relates to the use of a bispecific antibody comprising at least one binding portion which binds to human CD47 and at least two binding portions which bind to human CD38 to treat Acute myeloid leukemia (AML) or T-cell acute lymphoblastic leukemia (T-ALL).

Specifically, the present invention relates to the use of a bispecific antibody that has at least two CD38 binding portions which are biparatopic to treat Acute myeloid leukemia (AML) or T-cell acute lymphoblastic leukemia (T-ALL).

Specifically, the present invention relates the use of a bispecific antibody that has at least one binding portion which binds to human CD38 comprises a CDR set selected from the group comprising: SEQ ID NO: 103, SEQ ID NO: 163, SEQ ID NO: 223; SEQ ID NO: 117, SEQ ID NO: 177, SEQ ID NO: 237; SEQ ID NO: 121; SEQ ID NO: 181, SEQ ID NO: 241, to treat Acute myeloid leukemia (AML) or T-cell acute lymphoblastic leukemia (T-ALL).

Specifically, the present invention relates to the use of a bispecific antibody that has at least one binding portion which binds to human CD38 comprises a CDR set comprising the amino acid sequence of SEQ ID NO: 117, SEQ ID NO: 177, SEQ ID NO: 237; and a CDR set comprising the amino acid sequence of SEQ ID NO: 121; SEQ ID NO: 181, SEQ ID NO: 241, to treat Acute myeloid leukemia (AML) or T-cell acute lymphoblastic leukemia (T-ALL).

Specifically, the present invention relates to the use of a bispecific antibody that has at least one binding portion which binds to human CD47 comprises a CDR set comprising the amino acid sequence of SEQ ID NO: 75, SEQ ID NO: 135, SEQ ID NO: 195, to treat Acute myeloid leukemia (AML) or T-cell acute lymphoblastic leukemia (T-ALL).

In certain embodiments, the bispecific antibody according to the present invention comprises a Fc region.

In particular, said Fc region is a variant which comprises at least one amino acid modification relative to the Fc region of the parent antibody, whereas the antibody comprising the variant Fc region exhibits altered effector function compared to the parent antibody.

In certain embodiments, the bispecific antibody according to the present invention comprises at least one binding portion which binds to human CD47 that has an affinity to human CD47 lower than the affinity of at least one binding portion which binds to human CD38 has to human CD38.

The present invention also relates to an epitope on the human CD38 extracellular domain which is bound by the bispecific antibody disclosed herein.

The present invention also relates to an epitope on the human CD47 extracellular domain which is bound by the bispecific antibody disclosed herein.

The present invention also relates to an isolated nucleic acid encoding the bispecific antibody disclosed herein.

The present invention also relates to a vector comprising an isolated nucleic acid encoding the bispecific antibody disclosed herein.

The present invention also relates to a host cell comprising an isolated nucleic acid encoding the bispecific antibody disclosed herein or a vector comprising an isolated nucleic acid encoding the bispecific antibody disclosed herein.

The present invention also relates to a composition comprising the bispecific antibody according to the present invention and a pharmaceutically acceptable carrier, and to said composition further comprising another pharmaceutically active agent.

The present invention also relates to an immunoconjugate comprising the bispecific antibody according to the present invention linked to a therapeutic agent.

The present invention also relates to a pharmaceutical formulation comprising the antibody for use in the treatment of a subject having Acute myeloid leukemia (AML) or T-cell acute lymphoblastic leukemia (T-ALL) and a pharmaceutically acceptable carrier.

As utilized in accordance with the present disclosure, the following terms, unless otherwise indicated, shall be understood to have the following meanings. Unless otherwise required by context, singular terms shall include pluralities and plural terms shall include the singular. As used in this specification and the appended claims, the singular forms “a”, “an” and “the” include plural referents unless the content clearly dictates otherwise. Thus, for example, reference to “a molecule” optionally includes a combination of two or more such molecules, and the like.

It is understood that aspects and embodiments of the present disclosure described herein include “comprising,” “consisting,” and “consisting essentially of aspects and embodiments.

The term “polynucleotide” as used herein refers to single-stranded or double-stranded nucleic acid polymers of at least 10 nucleotides in length. In certain embodiments, the nucleotides comprising the polynucleotide can be ribonucleotides or deoxyribonucleotides or a modified form of either type of nucleotide. Such modifications include base modifications such as bromuridine, ribose modifications such as arabinoside and 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate. The term “polynucleotide” specifically includes single-stranded and double-stranded forms of DNA.

An “isolated polynucleotide” is a polynucleotide of genomic, cDNA, or synthetic origin or some combination thereof, which: (1) is not associated with all or a portion of a polynucleotide in which the isolated polynucleotide is found in nature, (2) is linked to a polynucleotide to which it is not linked in nature, or (3) does not occur in nature as part of a larger sequence.

An “isolated polypeptide” is one that: (1) is free of at least some other polypeptides with which it would normally be found, (2) is essentially free of other polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a polypeptide with which the “isolated polypeptide” is associated in nature, (6) is operably associated (by covalent or noncovalent interaction) with a polypeptide with which it is not associated in nature, or (7) does not occur in nature.

Such an isolated polypeptide can be encoded by genomic DNA, cDNA, mRNA or other RNA, of synthetic origin, or any combination thereof. Preferably, the isolated polypeptide is substantially free from polypeptides or other contaminants that are found in its natural environment that would interfere with its use (therapeutic, diagnostic, prophylactic, research or otherwise).

The term “antibody” as referred to herein includes whole antibodies and any antigen binding fragments or single chains thereof. Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (VH) and three constant domains (CH1, CH2, and CH3), wherein the VH domain is at the amino-terminus of the polypeptide and the CH3domain is at the carboxyl-terminus, and a full-length light chain immunoglobulin polypeptide includes a variable domain (VL) and a constant domain (CL), wherein the VL domain is at the amino-terminus of the polypeptide and the CL domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al, SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol Biol. 196: 901-17; Chothia et al, 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. Identification of predicted CDRs using the amino acid sequence is well known in the field, such as in Martin, A. C. “Protein sequence and structure analysis of antibody variable domains,” In Antibody Engineering, Vol. 2. Kontermann R., Diibel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments. All such alternative definitions are encompassed by the current invention and the sequences provided in this specification are not intended to exclude alternatively defined CDR sequences which may only comprise a portion of the CDR sequences provided in the sequence listing

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins. Fc molecules are made up of monomeric polypeptides that can be linked into dimeric or multimeric forms by covalent {i.e., disulfide bonds) and non-covalent association. The number of intermolecular disulfide bonds between monomeric subunits of native Fc molecules ranges from 1 to 4 depending on class {e.g., IgG, IgA, and IgE) or subclass {e.g., IgG1, IgG2, IgG3, IgAI, IgGA2, and IgG4). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the VH and CH1 domains of one heavy chain, wherein the VH-CH1 heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a CH1 domain.

A F(ab′) fragment typically includes one light chain and a portion of one heavy chain that contains more of the constant region (between the CH1 and CH2 domains), such that an interchain disulfide bond can be formed between two heavy chains to form a F(ab′)2molecule.

The term “binding protein” as used herein refers to a non-naturally occurring or recombinant or engineered molecule, such as non-naturally occurring or recombinant or engineered antibody, that specifically binds to at least one target antigen, e.g., a CD38 polypeptide, or a CD47 polypeptide of the present disclosure.

A “recombinant” molecule is one that has been prepared, expressed, created, or isolated by recombinant means.

One embodiment of the disclosure provides binding proteins having biological and immunological specificity to between one and three target antigens. Another embodiment of the disclosure provides nucleic acid molecules comprising nucleotide sequences encoding polypeptide chains that form such binding proteins. Another embodiment of the disclosure provides expression vectors comprising nucleic acid molecules comprising nucleotide sequences encoding polypeptide chains that form such binding proteins. Yet another embodiment of the disclosure provides host cells that express such binding proteins (i.e., comprising nucleic acid molecules or vectors encoding polypeptide chains that form such binding proteins).

The term “antigen” or “target antigen” or “antigen target” as used herein refers to a molecule or a portion of a molecule that is capable of being bound by a binding protein, and additionally is capable of being used in an animal to produce antibodies capable of binding to an epitope of that antigen. A target antigen may have one or more epitopes. With respect to each target antigen recognized by a binding protein, the binding protein is capable of competing with an intact antibody that recognizes the target antigen. The antigen is bound by a binding protein, such as an antibody, via an antigen binding site, also referred herein as “binding portion” or “binding domain”.

“CD38” is cluster of differentiation 38 polypeptide, a glycoprotein found on the surface of many immune cells. In some embodiments, a binding protein of the present disclosure binds the extracellular domain of one or more CD38 polypeptide. Exemplary CD38 extracellular domain polypeptide sequences include, but are not limited to, the extracellular domain of human CD38 (e.g., as represented by SEQ ID NO: 5) and the extracellular domain of cynomolgus monkey CD38 (e.g., as represented by SEQ ID NO: 6).

“CD47” is cluster of differentiation 47, a transmembrane protein that in humans is encoded by the CD47 gene. CD47 belongs to the immunoglobulin superfamily, it partners with membrane integrins and also binds the ligands thrombospondin-1 (TSP-1) and signal-regulatory protein alpha (SIRPα). CD47 acts as a don't eat me signal to macrophages of the immune system. Human CD47 is encoded by SEQ ID NO: 7 and cynomolgus monkey CD47 is encoded by SEQ ID NO: 8.

The term “monospecific binding protein” refers to a binding protein that specifically binds to one antigen target.

The term “monovalent binding protein” refers to a binding protein that has one antigen binding site.

The term “bispecific binding protein” refers to a binding protein that specifically binds to two different antigen targets. In some embodiments, a bispecific binding protein binds to two different antigens. In some embodiments, a bispecific binding protein binds to two different epitopes on the same antigen.

The term “bivalent binding protein” refers to a binding protein that has two antigen binding sites.

The term “trispecific binding protein” refers to a binding protein that specifically binds to three different antigen targets. In some embodiments, a trispecific binding protein binds to three different antigens. In some embodiments, a trispecific binding protein binds to one, two, or three different epitopes on the same antigen.

The term “trivalent binding protein” refers to a binding protein that has three binding sites. In particular embodiments the trivalent binding protein can bind to one antigen target (i.e. monospecific trivalent binding protein”). In other embodiments, the trivalent binding protein can bind to two antigen targets (i.e. bispecific trivalent binding protein”). In other embodiments, the trivalent binding protein can bind to three antigen targets (i.e. trispecific trivalent binding protein”). In a more particular embodiment, the trivalent binding protein that bind to two antigen targets comprises a binding portion that binds to a first antigen target and two binding portions that bind to the same epitope of a second antigen target; such binding protein is referred to as “monoparatopic”. In another more particular embodiment, the trivalent binding protein that bind to two antigen targets comprises a binding portion that binds to a first antigen target and two binding portions that bind to two distinct epitopes of a second antigen target; such binding protein is referred to as “biparatopic”. An “isolated” binding protein is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes. In some embodiments, the binding protein will be purified: (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding proteins include the binding protein in situ within recombinant cells since at least one component of the binding protein's natural environment will not be present.

The terms “substantially pure” or “substantially purified” as used herein refer to a compound or species that is the predominant species present {i.e., on a molar basis it is more abundant than any other individual species in the composition). In some embodiments, a substantially purified fraction is a composition wherein the species comprises at least about 50%) (on a molar basis) of all macromolecular species present. In other embodiments, a substantially pure composition will comprise more than about 80%>, 85%>, 90%, 95%, or 99% of all macromolar species present in the composition. In still other embodiments, the species is purified to essential homogeneity (contaminant species cannot be detected in the composition by conventional detection methods) wherein the composition consists essentially of a single macromolecular species.

A nucleic acid is “isolated” or “substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art, see e.g. F. Ausubel, et al., ed. (1987) Current Protocols in Molecular Biology, Greene Publishing and Wiley Interscience, New York. A nucleic acid of the invention can be, for example, DNA or RNA and may or may not contain intron sequences.

The term “epitope” includes any determinant, preferably a polypeptide determinant, capable of specifically binding to an immunoglobulin or T-cell receptor. In certain embodiments, epitope determinants include chemically active surface groupings of molecules such as amino acids, sugar side chains, phosphoryl groups, or sulfonyl groups, and, in certain embodiments, may have specific three-dimensional structural characteristics and/or specific charge characteristics. An epitope is a region of an antigen that is bound by an antibody or binding protein. In certain embodiments, a binding protein is said to specifically bind an antigen when it preferentially recognizes its target antigen in a complex mixture of proteins and/or macromolecules. In some embodiments, a binding protein is said to specifically bind an antigen when the equilibrium dissociation constant is <10⁸M, more preferably when the equilibrium dissociation constant is <10⁹M, and most preferably when the dissociation constant is <10¹⁰M.

The dissociation constant (KD) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the BIAcore system (GE). Surface plasmon analysis can also be performed by immobilizing the analyte (binding protein on a biosensor matrix) and presenting the ligand (target antigen). The term “KD,” as used herein refers to the dissociation constant of the interaction between a particular binding protein and a target antigen.

The term “binds to” as used herein in reference to a binding protein refers to the ability of a binding protein or an antigen-binding fragment thereof to bind to an antigen containing an epitope with an KD of at least about 1×10⁻⁶M, 1×10⁻⁷M, 1×10⁻⁸M, 1×10⁻⁹M, 1×10⁻¹⁰M, 1×10⁻¹¹ M, 1×10⁻²M, or less and/or to bind to an epitope with an affinity that is at least two-fold greater than its affinity for a nonspecific antigen. In some embodiments, a binding protein of the present disclosure binds to two or more antigens, e.g., a human and a cynomolgus monkey CD38 polypeptide.

In some embodiments, an antigen binding domain and/or binding protein of the present disclosure “cross reacts” with human and cynomolgus monkey CD38 polypeptides, e.g., CD38 extracellular domains, such as SEQ ID NO: 295 (human CD38 isoform 28907-1), SEQ ID NO: 294 (human CD38 isoform 28907-2), SEQ ID NO: 296 (human CD38 isoform 28907-E) and SEQ ID NO:6 (cynomolgus monkey CD38). In some embodiments, a binding protein or antigen-binding fragment thereof cross-reacts with human CD47 (e.g., SEQ ID NO: 300 (human CD47 isoform OA3-323), SEQ ID NO: 301 (human CD47 isoform OA3-293), SEQ ID NO: 302 (human CD47 isoform OA3-305), and SEQ ID NO: 303 (human CD47 isoform OA3-312)) and cynomolgus monkey CD47. A binding protein binding to antigen 1 is “cross-reactive” to antigen 2 when the EC50S are in a similar range for both antigens.

The term “linker” as used herein refers to one or more amino acid residues inserted between immunoglobulin domains to provide sufficient mobility for the domains of the light and heavy chains to fold into cross over dual variable region immunoglobulins. A linker is inserted at the transition between variable domains or between variable and constant domains, respectively, at the sequence level. The transition between domains can be identified because the approximate size of the immunoglobulin domains are well understood. The precise location of a domain transition can be determined by locating peptide stretches that do not form secondary structural elements such as beta-sheets or alpha-helices as demonstrated by experimental data or as can be assumed by techniques of modeling or secondary structure prediction.

The term “vector” as used herein refers to any molecule (e.g., nucleic acid, plasmid, or virus) that is used to transfer coding information to a host cell. The term “vector” includes a nucleic acid molecule that is capable of transporting another nucleic acid to which it has been linked. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms “plasmid” and “vector” may be used interchangeably herein, as a plasmid is the most commonly used form of vector. However, the disclosure is intended to include other forms of expression vectors, such as viral vectors (e.g., replication defective retroviruses, adenoviruses, and adeno-associated viruses), which serve equivalent functions.

The phrase “recombinant host cell” (or “host cell”) as used herein refers to a cell into which a recombinant expression vector has been introduced. A recombinant host cell or host cell is intended to refer not only to the particular subject cell, but also to the progeny of such a cell. Because certain modifications may occur in succeeding generations due to either mutation or environmental influences, such progeny may not, in fact, be identical to the parent cell, but such cells are still included within the scope of the term “host cell” as used herein. A wide variety of host cell expression systems can be used to express the binding proteins, including bacterial, yeast, baculoviral, and mammalian expression systems (as well as phage display expression systems). An example of a suitable bacterial expression vector is pUC19. To express a binding protein recombinantly, a host cell is transformed or transfected with one or more recombinant expression vectors carrying DNA fragments encoding the polypeptide chains of the binding protein such that the polypeptide chains are expressed in the host cell and, preferably, secreted into the medium in which the host cells are cultured, from which medium the binding protein can be recovered.

The term “transformation” as used herein refers to a change in a cell's genetic characteristics, and a cell has been transformed when it has been modified to contain a new DNA. For example, a cell is transformed where it is genetically modified from its native state. Following transformation, the transforming DNA may recombine with that of the cell by physically integrating into a chromosome of the cell, or may be maintained transiently as an episomal element without being replicated, or may replicate independently as a plasmid. A cell is considered to have been stably transformed when the DNA is replicated with the division of the cell. The term “transfection” as used herein refers to the uptake of foreign or exogenous DNA by a cell, and a cell has been “transfected” when the exogenous DNA has been introduced inside the cell membrane. A number of transfection techniques are well known in the art. Such techniques can be used to introduce one or more exogenous DNA molecules into suitable host cells.

The term “naturally occurring” as used herein and applied to an object refers to the fact that the object can be found in nature and has not been manipulated by man. For example, a polynucleotide or polypeptide that is present in an organism (including viruses) that can be isolated from a source in nature and that has not been intentionally modified by man is naturally-occurring. Similarly, “non-naturally occurring” as used herein refers to an object that is not found in nature or that has been structurally modified or synthesized by man.

As used herein, the twenty conventional amino acids and their abbreviations follow conventional usage. Stereoisomers (e.g., D-amino acids) of the twenty conventional amino acids; unnatural amino acids and analogs such as α-, α-di substituted amino acids, N-alkyl amino acids, lactic acid, and other unconventional amino acids may also be suitable components for the polypeptide chains of the binding proteins. Examples of unconventional amino acids include: 4-hydroxyproline, γ-carboxyglutamate, ε-N,N,N-trimethyllysine, ε-N-acetyllysine, O-phosphoserine, N-acetyl serine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxyl-terminal direction, in accordance with standard usage and convention.

Naturally occurring residues may be divided into classes based on common side chain properties:
(1) hydrophobic: Met, Ala, Val, Leu, Ile, Phe, Trp, Tyr, Pro;
(2) polar hydrophilic: Arg, Asn, Asp, Gln, Glu, His, Lys, Ser, Thr;
(3) aliphatic: Ala, Gly, Ile, Leu, Val, Pro;
(4) aliphatic hydrophobic: Ala, Ile, Leu, Val, Pro;
(5) neutral hydrophilic: Cys, Ser, Thr, Asn, Gln;
(6) acidic: Asp, Glu;
(7) basic: His, Lys, Arg;
(8) residues that influence chain orientation: Gly, Pro;
(9) aromatic: His, Trp, Tyr, Phe; and
(10) aromatic hydrophobic: Phe, Trp, Tyr.

Conservative amino acid substitutions may involve exchange of a member of one of these classes with another member of the same class. Non-conservative substitutions may involve the exchange of a member of one of these classes for a member from another class.

A skilled artisan will be able to determine suitable variants of the polypeptide chains of the binding proteins using well-known techniques. For example, one skilled in the art may identify suitable areas of a polypeptide chain that may be changed without destroying activity by targeting regions not believed to be important for activity. Alternatively, one skilled in the art can identify residues and portions of the molecules that are conserved among similar polypeptides. In addition, even areas that may be important for biological activity or for structure may be subject to conservative amino acid substitutions without destroying the biological activity or without adversely affecting the polypeptide structure.

The term “patient” as used herein includes human and animal subjects.

The terms “treatment” or “treat” as used herein refer to both therapeutic treatment and prophylactic or preventative measures. Those in need of treatment include those having a disorder as well as those prone to have the disorder or those in which the disorder is to be prevented. In particular embodiments, binding proteins can be used to treat humans with cancer, or humans susceptible to cancer, or ameliorate cancer in a human subject. The binding proteins can also be used to prevent cancer in a human patient. In particular embodiments, the cancer is multiple myeloma, acute lymphoblastic leukemia, chronic lymphocytic leukemia, acute myeloid leukemia, lymphoma, breast cancer such as Her2+ breast cancer, prostate cancer, cervival cancer, germinal center B-cell lympohoma or B-cell acute lymphoblastic leukemia, Chronic lymphocytic leukemia (CLL), Non-Hodgkin lymphoma, diffuse large B-cell lymphoma, non small cell lung cancer (NSCLC), Hepatocellular carcinoma (HCC), High-grade serous ovarian carcinoma, peritoneal cancer.

The terms “pharmaceutical composition” or “therapeutic composition” as used herein refer to a compound or composition capable of inducing a desired therapeutic effect when properly administered to a patient.

The term “pharmaceutically acceptable carrier” or “physiologically acceptable carrier” as used herein refers to one or more formulation materials suitable for accomplishing or enhancing the delivery of a binding protein.

The terms “effective amount” and “therapeutically effective amount” when used in reference to a pharmaceutical composition comprising one or more binding proteins refer to an amount or dosage sufficient to produce a desired therapeutic result. More specifically, a therapeutically effective amount is an amount of a binding protein sufficient to inhibit, for some period of time, one or more of the clinically defined pathological processes associated with the condition being treated. The effective amount may vary depending on the specific binding protein that is being used, and also depends on a variety of factors and conditions related to the patient being treated and the severity of the disorder. For example, if the binding protein is to be administered in vivo, factors such as the age, weight, and health of the patient as well as dose response curves and toxicity data obtained in preclinical animal work would be among those factors considered. The determination of an effective amount or therapeutically effective amount of a given pharmaceutical composition is well within the ability of those skilled in the art.

One embodiment of the disclosure provides a pharmaceutical composition comprising a pharmaceutically acceptable carrier and a therapeutically effective amount of a binding protein.

### Anti-CD38 Binding Proteins

Certain aspects of the present disclosure relate to binding proteins that comprise an antigen binding site that binds a CD38 polypeptide {e.g., human and cynomolgus monkey CD38 polypeptides). In some embodiments, the binding proteins are monospecific and/or monovalent, bispecific and/or bivalent, trispecific and/or trivalent, or multispecific and/or multivalent.

A variety of features of exemplary monospecific, bispecific, or trispecific binding proteins are described herein. For example, in some embodiments, a binding protein or antigen-binding fragment thereof cross-reacts with human CD38 (e.g., SEQ ID NO: 295 (human CD38 isoform 28907-1), SEQ ID NO: 294 (human CD38 isoform 28907-2), SEQ ID NO: 296 (human CD38 isoform 28907-E)) and cynomolgus monkey CD38. In some embodiments, a binding protein induces apoptosis of a CD38+ cell.

In some embodiments, the binding protein is bivalent and/or bispecific. In some embodiments, the binding protein is multivalent, such as trivalent or tetravalent. In some embodiments, at least one of the antigen binding sites binds a CD38 polypeptide (e.g., the extracellular domain of human and/or cynomolgus monkey CD38 polypeptides).

In any of the bispecific binding proteins described supra, the target antigen other than CD38 can be any of the following exemplary antigen targets: A2AR, APRIL, ATPDase, BAFF, BAFFR, BCMA, BIYS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-la), CCL4 (also known as MIP-Ib), CCL5 (also known as RANTES), CCL7 (also known as MCP-3), CCL8 (also known as mcp-2), CCL11 (also known as eotaxin), CCL15 (also known as MIP-Id), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CCR7, CD19, CD20, CD23 (also known as FCER2, a receptor for IgE), CD24, CD27, CD28, CD38, CD39, CD40, CD47, CD48, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDLI), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, GPRC5D, Her2, HHLA2, HMGB1, HVEM, ICOSLG, IDO, IFNa, IgE, IGF1R, IL2Rbeta, IL1, ILIRAP, ILILIA, IL1B, IL1F10, IL2, IL4, IL4Ra, IL5, IL5R, IL6, IL7, IL7Ra, I18, IL9, RAP, IL9R, IL10, rIL10, IL12, IL13, IL13Ral, IL13Ra2, IL15, IL17, IL17Rb (also known as a receptorfor IL25), IL18, IL22, IL23, IL25, IL27, IL33, IL35, ITGB4 (also known as b4 integrin), ITK, KIR, LAG3, LAMP1, leptin, LPFS2, MHC class II, NCR3LG1, KG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1), XCL1 and XCL2. In some embodiments, one or more of the above antigen targets are human antigen targets.

In any of the bispecific binding proteins described supra, any linker or combination of linkers described herein may be used.

In certain particular embodiments, the present invention relates to a bispecific antibody comprising at least one binding portion which binds to human CD38 comprising a CDR set selected from the group comprising: SEQ ID NO: 80, SEQ ID NO: 140, SEQ ID NO: 200; SEQ ID NO: 81, SEQ ID NO: 141, SEQ ID NO: 201; SEQ ID NO: 82, SEQ ID NO: 142, SEQ ID NO: 202; SEQ ID NO: 83, SEQ ID NO: 143, SEQ ID NO: 203; SEQ ID NO: 84, SEQ ID NO: 144, SEQ ID NO: 204; SEQ ID NO: 85, SEQ ID NO: 145, SEQ ID NO: 205; SEQ ID NO: 86, SEQ ID NO: 146, SEQ ID NO: 206; SEQ ID NO: 87, SEQ ID NO: 147, SEQ ID NO: 207, SEQ ID NO: 88, SEQ ID NO: 148, SEQ ID NO: 208; SEQ ID NO: 89, SEQ ID NO: 149, SEQ ID NO: 209; SEQ ID NO: 90, SEQ ID NO: 150, SEQ ID NO: 210; SEQ ID NO: 91, SEQ ID NO: 151, SEQ ID NO: 211; SEQ ID NO: 92, SEQ ID NO: 152, SEQ ID NO: 212; SEQ ID NO: 93, SEQ ID NO: 153, SEQ ID NO: 213; SEQ ID NO: 94, SEQ ID NO: 154, SEQ ID NO: 214; SEQ ID NO: 95, SEQ ID NO: 155, SEQ ID NO: 215; SEQ ID NO: 96, SEQ ID NO: 156, SEQ ID NO: 216; SEQ ID NO: 97, SEQ ID NO: 157, SEQ ID NO: 217; SEQ ID NO: 98, SEQ ID NO: 158, SEQ ID NO: 218; SEQ ID NO: 99, SEQ ID NO: 159, SEQ ID NO: 219; SEQ ID NO: 100, SEQ ID NO: 160, SEQ ID NO: 220; SEQ ID NO: 101, SEQ ID NO: 161, SEQ ID NO: 221; SEQ ID NO: 102, SEQ ID NO: 162, SEQ ID NO: 222; SEQ ID NO: 103, SEQ ID NO: 163, SEQ ID NO: 223; SEQ ID NO: 104, SEQ ID NO: 164, SEQ ID NO: 224; SEQ ID NO: 105, SEQ ID NO: 165, SEQ ID NO: 225; SEQ ID NO: 106, SEQ ID NO: 166, SEQ ID NO: 226; SEQ ID NO: 107, SEQ ID NO: 167, SEQ ID NO: 227; SEQ ID NO: 108, SEQ ID NO: 168, SEQ ID NO: 228; SEQ ID NO: 109, SEQ ID NO: 169, SEQ ID NO: 229; SEQ ID NO: 110, SEQ ID NO: 170, SEQ ID NO: 230; SEQ ID NO: 111, SEQ ID NO: 171, SEQ ID NO: 231; SEQ ID NO: 112, SEQ ID NO: 172, SEQ ID NO: 232; SEQ ID NO: 113, SEQ ID NO: 173, SEQ ID NO: 233, SEQ ID NO: 114, SEQ ID NO: 174, SEQ ID NO: 234; SEQ ID NO: 115, SEQ ID NO: 176, SEQ ID NO: 235; SEQ ID NO: 116, SEQ ID NO: 175, SEQ ID NO: 236; SEQ ID NO: 117, SEQ ID NO: 177, SEQ ID NO: 237; SEQ ID NO: 118, SEQ ID NO: 178, SEQ ID NO: 238; SEQ ID NO: 119, SEQ ID NO: 179, SEQ ID NO: 239; SEQ ID NO: 120, SEQ ID NO: 180, SEQ ID NO: 240; SEQ ID NO: 121; SEQ ID NO: 181, SEQ ID NO: 241; SEQ ID NO: 122, SEQ ID NO: 182, SEQ ID NO: 242; SEQ ID NO: 123, SEQ ID NO: 183, SEQ ID NO: 243; SEQ ID NO: 124, SEQ ID NO: 184, SEQ ID NO: 244; SEQ ID NO: 125, SEQ ID NO: 185, SEQ ID NO: 245; SEQ ID NO: 126, SEQ ID NO: 186, SEQ ID NO: 246; SEQ ID NO: 127, SEQ ID NO: 187, SEQ ID NO: 247; SEQ ID NO: 128, SEQ ID NO: 188, SEQ ID NO: 248; SEQ ID NO: 129, SEQ ID NO: 189, SEQ ID NO: 249; SEQ ID NO: 130, SEQ ID NO: 190, SEQ ID NO: 250. In a preferred embodiment, the bispecific antibody according to the present invention comprises at least one binding portion which binds to human CD38 comprising a CDR set selected from the group comprising SEQ ID NO: 112, SEQ ID NO: 172, SEQ ID NO: 232; SEQ ID NO: 115, SEQ ID NO: 176, SEQ ID NO: 235; SEQ ID NO: 117, SEQ ID NO: 177, SEQ ID NO: 237; SEQ ID NO: 103, SEQ ID NO: 163, SEQ ID NO: 223, and SEQ ID NO: 121; SEQ ID NO: 181, SEQ ID NO: 241.

In preferred aspects, the antibody for use disclosed herein further comprises a common light chain. More in particular, the bispecific antibody for use disclosed hereon further comprises a common light chain comprising an amino acid sequence of SEQ ID NO: 10. In another preferred aspects, the antibody for use disclosed herein further comprises a common light chain CDRs set comprising SEQ ID NO: 304, 305, and 306.

### Anti-CD47 Binding Proteins

Certain aspects of the present disclosure relate to binding proteins that comprise an antigen binding site that binds a CD47 polypeptide {e.g., human and cynomolgus monkey CD47 polypeptides). In some embodiments, the binding proteins are monospecific and/or monovalent, bispecific and/or bivalent, trispecific and/or trivalent, or multispecific and/or multivalent.

A variety of features of exemplary monospecific, bispecific, or trispecific binding proteins are described herein. For example, in some embodiments, a binding protein or antigen-binding fragment thereof cross-reacts with human CD47 (e.g., SEQ ID NO: 300 (human CD47 isoform OA3-323), SEQ ID NO: 301 (human CD47 isoform OA3-293), SEQ ID NO: 302 (human CD47 isoform OA3-305), and SEQ ID NO: 303 (human CD47 isoform OA3-312)) and cynomolgus monkey CD47.

In some embodiments, the binding protein is bivalent and/or bispecific. In some embodiments, the binding protein is multivalent, such as trivalent or tetravalent. In some embodiments, at least one of the antigen binding sites binds a CD47 polypeptide (e.g., the extracellular domain of human and/or cynomolgus monkey CD47 polypeptides).

In any of the bispecific binding proteins described supra, the target antigen other than CD47 can be any of the following exemplary antigen targets: A2AR, APRIL, ATPDase, BAFF, BAFFR, BCMA, BIYS, BTK, BTLA, B7DC, B7H1, B7H4 (also known as VTCN1), B7H5, B7H6, B7H7, B7RP1, B7-4, C3, C5, CCL2 (also known as MCP-1), CCL3 (also known as MIP-la), CCL4 (also known as MIP-Ib), CCL5 (also known as RANTES), CCL7 (also known as MCP-3), CCL8 (also known as mcp-2), CCL11 (also known as eotaxin), CCL15 (also known as MIP-Id), CCL17 (also known as TARC), CCL19 (also known as MIP-3b), CCL20 (also known as MIP-3a), CCL21 (also known as MIP-2), CCL24 (also known as MPIF-2/eotaxin-2), CCL25 (also known as TECK), CCL26 (also known as eotaxin-3), CCR3, CCR4, CD3, CCR7, CD19, CD20, CD23 (also known as FCER2, a receptor for IgE), CD24, CD27, CD28, CD38, CD39, CD40, CD47, CD48, CD70, CD80 (also known as B7-1), CD86 (also known as B7-2), CD122, CD137 (also known as 41BB), CD137L, CD152 (also known as CTLA4), CD154 (also known as CD40L), CD160, CD272, CD273 (also known as PDL2), CD274 (also known as PDLI), CD275 (also known as B7H2), CD276 (also known as B7H3), CD278 (also known as ICOS), CD279 (also known as PD-1), CDH1 (also known as E-cadherin), chitinase, CLEC9, CLEC91, CRTH2, CSF-1 (also known as M-CSF), CSF-2 (also known as GM-CSF), CSF-3 (also known as GCSF), CX3CL1 (also known as SCYD1), CXCL12 (also known as SDF1), CXCL13, CXCR3, DNGR-1, ectonucleoside triphosphate diphosphohydrolase 1, EGFR, ENTPD1, FCER1A, FCER1, FLAP, FOLH1, Gi24, GITR, GITRL, GM-CSF, GPRC5D, Her2, HHLA2, HMGB1, HVEM, ICOSLG, IDO, IFNa, IgE, IGF1R, IL2Rbeta, IL1, ILIRAP, ILILIA, IL1B, IL1F10, IL2, IL4, IL4Ra, IL5, IL5R, IL6, IL7, IL7Ra, I18, IL9, RAP, IL9R, IL10, rIL10, IL12, IL13, IL13Ral, IL13Ra2, IL15, IL17, IL17Rb (also known as a receptorfor IL25), I18, IL22, IL23, IL25, IL27, IL33, IL35, ITGB4 (also known as b4 integrin), ITK, KIR, LAG3, LAMP1, leptin, LPFS2, MHC class II, NCR3LG1, KG2D, NTPDase-1, OX40, OX40L, PD-1H, platelet receptor, PROM1, S152, SISP1, SLC, SPG64, ST2 (also known as a receptor for IL33), STEAP2, Syk kinase, TACI, TDO, T14, TIGIT, TIM3, TLR, TLR2, TLR4, TLR5, TLR9, TMEF1, TNFa, TFRSF7, Tp55, TREM1, TSLP (also known as a co-receptor for IL7Ra), TSLPR, TWEAK, VEGF, VISTA, Vstm3, WUCAM, and XCR1 (also known as GPR5/CCXCR1), XCL1 and XCL2. In some embodiments, one or more of the above antigen targets are human antigen targets.

In certain particular embodiments, the present invention relates to a bispecific antibody comprising a binding portion which binds to human CD47 comprising a CDR set selected from the group comprising: SEQ ID NO: 71, SEQ ID NO: 131, SEQ ID NO: 191; SEQ ID NO: 72, SEQ ID NO: 132, SEQ ID NO: 192; SEQ ID NO: 73, SEQ ID NO: 133, SEQ ID NO: 193; SEQ ID NO: 74, SEQ ID NO: 134, SEQ ID NO: 194; SEQ ID NO: 75, SEQ ID NO: 135, SEQ ID NO: 195; SEQ ID NO: 76, SEQ ID NO: 136, SEQ ID NO: 196; SEQ ID NO: 77, SEQ ID NO: 137, SEQ ID NO: 197; SEQ ID NO: 78, SEQ ID NO: 138, SEQ ID NO: 198; SEQ ID NO: 79, SEQ ID NO: 139, SEQ ID NO: 199; SEQ ID NO: 80, SEQ ID NO: 140, SEQ ID NO: 200; SEQ ID NO: 81, SEQ ID NO: 141, SEQ ID NO: 201. In a preferred embodiment, the bispecific antibody according to the present invention comprises a binding portion which binds to human CD47 comprising a CDR set comprising SEQ ID NO: 75, SEQ ID NO: 135, SEQ ID NO: 195.

In preferred aspects, the antibody for use disclosed herein further comprises a common light chain. More in particular, the bispecific antibody for use disclosed hereon further comprises a common light chain comprising an amino acid sequence of SEQ ID NO: 10. In another preferred aspects, the antibody for use disclosed herein further comprises a common light chain CDRs set comprising SEQ ID NO: 304, 305, and 306.

### Bispecific Anti-CD47 and Anti-CD38 Proteins

The present invention relates to a bispecific antibody comprising at least two binding portions, at least one of which binds to human CD38 and at least one of which binds to human CD47.

In particular the at least one binding portion which binds to human CD38 comprises a CDR set selected from the group comprising: SEQ ID NO: 80, SEQ ID NO: 140, SEQ ID NO: 200; SEQ ID NO: 81, SEQ ID NO: 141, SEQ ID NO: 201; SEQ ID NO: 82, SEQ ID NO: 142, SEQ ID NO: 202; SEQ ID NO: 83, SEQ ID NO: 143, SEQ ID NO: 203; SEQ ID NO: 84, SEQ ID NO: 144, SEQ ID NO: 204; SEQ ID NO: 85, SEQ ID NO: 145, SEQ ID NO: 205; SEQ ID NO: 86, SEQ ID NO: 146, SEQ ID NO: 206; SEQ ID NO: 87, SEQ ID NO: 147, SEQ ID NO: 207, SEQ ID NO: 88, SEQ ID NO: 148, SEQ ID NO: 208; SEQ ID NO: 89, SEQ ID NO: 149, SEQ ID NO: 209; SEQ ID NO: 90, SEQ ID NO: 150, SEQ ID NO: 210; SEQ ID NO: 91, SEQ ID NO: 151, SEQ ID NO: 211; SEQ ID NO: 92, SEQ ID NO: 152, SEQ ID NO: 212; SEQ ID NO: 93, SEQ ID NO: 153, SEQ ID NO: 213; SEQ ID NO: 94, SEQ ID NO: 154, SEQ ID NO: 214; SEQ ID NO: 95, SEQ ID NO: 155, SEQ ID NO: 215; SEQ ID NO: 96, SEQ ID NO: 156, SEQ ID NO: 216; SEQ ID NO: 97, SEQ ID NO: 157, SEQ ID NO: 217; SEQ ID NO: 98, SEQ ID NO: 158, SEQ ID NO: 218; SEQ ID NO: 99, SEQ ID NO: 159, SEQ ID NO: 219; SEQ ID NO: 100, SEQ ID NO: 160, SEQ ID NO: 220; SEQ ID NO: 101, SEQ ID NO: 161, SEQ ID NO: 221; SEQ ID NO: 102, SEQ ID NO: 162, SEQ ID NO: 222; SEQ ID NO: 103, SEQ ID NO: 163, SEQ ID NO: 223; SEQ ID NO: 104, SEQ ID NO: 164, SEQ ID NO: 224; SEQ ID NO: 105, SEQ ID NO: 165, SEQ ID NO: 225; SEQ ID NO: 106, SEQ ID NO: 166, SEQ ID NO: 226; SEQ ID NO: 107, SEQ ID NO: 167, SEQ ID NO: 227; SEQ ID NO: 108, SEQ ID NO: 168, SEQ ID NO: 228; SEQ ID NO: 109, SEQ ID NO: 169, SEQ ID NO: 229; SEQ ID NO: 110, SEQ ID NO: 170, SEQ ID NO: 230; SEQ ID NO: 111, SEQ ID NO: 171, SEQ ID NO: 231; SEQ ID NO: 112, SEQ ID NO: 172, SEQ ID NO: 232; SEQ ID NO: 113, SEQ ID NO: 173, SEQ ID NO: 233, SEQ ID NO: 114, SEQ ID NO: 174, SEQ ID NO: 234; SEQ ID NO: 115, SEQ ID NO: 176, SEQ ID NO: 235; SEQ ID NO: 116, SEQ ID NO: 175, SEQ ID NO: 236; SEQ ID NO: 117, SEQ ID NO: 177, SEQ ID NO: 237; SEQ ID NO: 118, SEQ ID NO: 178, SEQ ID NO: 238; SEQ ID NO: 119, SEQ ID NO: 179, SEQ ID NO: 239; SEQ ID NO: 120, SEQ ID NO: 180, SEQ ID NO: 240; SEQ ID NO: 121; SEQ ID NO: 181, SEQ ID NO: 241; SEQ ID NO: 122, SEQ ID NO: 182, SEQ ID NO: 242; SEQ ID NO: 123, SEQ ID NO: 183, SEQ ID NO: 243; SEQ ID NO: 124, SEQ ID NO: 184, SEQ ID NO: 244; SEQ ID NO: 125, SEQ ID NO: 185, SEQ ID NO: 245; SEQ ID NO: 126, SEQ ID NO: 186, SEQ ID NO: 246; SEQ ID NO: 127, SEQ ID NO: 187, SEQ ID NO: 247; SEQ ID NO: 128, SEQ ID NO: 188, SEQ ID NO: 248; SEQ ID NO: 129, SEQ ID NO: 189, SEQ ID NO: 249; SEQ ID NO: 130, SEQ ID NO: 190, SEQ ID NO: 250; and the at least one binding portion which binds to human CD47 comprises a CDR set selected from the group comprising: SEQ ID NO: 71, SEQ ID NO: 131, SEQ ID NO: 191; SEQ ID NO: 72, SEQ ID NO: 132, SEQ ID NO: 192; SEQ ID NO: 73, SEQ ID NO: 133, SEQ ID NO: 193; SEQ ID NO: 74, SEQ ID NO: 134, SEQ ID NO: 194; SEQ ID NO: 75, SEQ ID NO: 135, SEQ ID NO: 195; SEQ ID NO: 76, SEQ ID NO: 136, SEQ ID NO: 196; SEQ ID NO: 77, SEQ ID NO: 137, SEQ ID NO: 197; SEQ ID NO: 78, SEQ ID NO: 138, SEQ ID NO: 198; SEQ ID NO: 79, SEQ ID NO: 139, SEQ ID NO: 199; SEQ ID NO: 80, SEQ ID NO: 140, SEQ ID NO: 200; SEQ ID NO: 81, SEQ ID NO: 141, SEQ ID NO: 201.

More specifically, the at least one binding portion which binds to human CD38 comprises a CDR set selected from the group comprising: SEQ ID NO: 112, SEQ ID NO: 172, SEQ ID NO: 232; SEQ ID NO: 115, SEQ ID NO: 176, SEQ ID NO: 235; SEQ ID NO: 117, SEQ ID NO: 177, SEQ ID NO: 237; SEQ ID NO: 103, SEQ ID NO: 163, SEQ ID NO: 223, and SEQ ID NO: 121; SEQ ID NO: 181, SEQ ID NO: 241; and the at least one binding portion which binds to human CD47 comprises a CDR set comprising SEQ ID NO: 75, SEQ ID NO: 135, SEQ ID NO: 195. Even more specifically, the at least one binding portion which binds to human CD38 comprises a CDR set comprising SEQ ID NO: 117, SEQ ID NO: 177, SEQ ID NO: 237; and the at least one binding portion which binds to human CD47 comprises a CDR set selected from the group comprising set comprising SEQ ID NO: 75, SEQ ID NO: 135, SEQ ID NO: 195.

In exemplary embodiments, the present invention relates to bispecific antibodies comprising at least one binding portion which binds to human CD47 and at least two binding portions which bind to human CD38. The at least one binding portion which binds to human CD47 may comprise any of the CDRs set described above and in paragraph “Anti-CD47 Binding Proteins”. The at least two binding portions which bind to human CD38 may comprise any of the CDRs set described above and in paragraph “Anti-CD38 Binding Proteins”.

In certain embodiments, the at least two CD38 binding portions are monoparatopic, i.e. they bind to the same epitope of their antigen target. In other embodiments, the at least two CD38 binding portions are biparatopic, i.e. they bind to different epitopes their antigen target. In particular, the antibody according to certain embodiments of the present invention comprises at least two binding portions which binds to human CD38: one Fc proximal binding portion which binds to a first epitope of human CD38, and one Fc distal binding portion which binds to a second epitope of human CD38. In particular embodiments, at least one of the at least two binding portions which binds to human CD38 comprises a CDR set selected from the group comprising SEQ ID NO: 112, SEQ ID NO: 172, SEQ ID NO: 232; SEQ ID NO: 115, SEQ ID NO: 176, SEQ ID NO: 235; SEQ ID NO: 117, SEQ ID NO: 177, SEQ ID NO: 237; SEQ ID NO: 103, SEQ ID NO: 163, SEQ ID NO: 223, and SEQ ID NO: 121; SEQ ID NO: 181, SEQ ID NO: 241. In more specific embodiments, at least one of the at least two binding portions which binds to human CD38 comprises a CDR set selected form the group comprising SEQ ID NO: 103, SEQ ID NO: 163, SEQ ID NO: 223, and SEQ ID NO: 121; SEQ ID NO: 181, SEQ ID NO: 241; and at least one of the at least two binding portions which binds to human CD38 comprises the CDR set SEQ ID NO: 117, SEQ ID NO: 177, SEQ ID NO: 237. In a preferred embodiment, the Fc proximal binding portion which binds to a first epitope of human CD38 comprises a CDR set selected from the group comprising SEQ ID NO: 103, SEQ ID NO: 163, SEQ ID NO: 223, and SEQ ID NO: 121; SEQ ID NO: 181, SEQ ID NO: 241; and the Fc distal binding portion which binds to a second epitope of human CD38 comprises the CDR set SEQ ID NO: 117, SEQ ID NO: 177, SEQ ID NO: 237. In a more preferred embodiment, the Fc proximal binding portion which binds to a first epitope of human CD38 comprises a CDR set selected form the group comprising SEQ ID NO: 103, SEQ ID NO: 163, SEQ ID NO: 223, and SEQ ID NO: 121; SEQ ID NO: 181, SEQ ID NO: 241; the Fc distal binding portion which binds to a second epitope of human CD38 comprises the CDR set SEQ ID NO: 117, SEQ ID NO: 177, SEQ ID NO: 237; further the bispecific antibody described above comprises at least one binding portion which binds to human CD47 comprising a CDR set comprising SEQ ID NO: 75, SEQ ID NO: 135, SEQ ID NO: 195. Preferably, the bispecific antibody of the present invention comprises at least one binding portion which binds to human CD38 comprises a CDR set comprising SEQ ID NO: 117, SEQ ID NO: 177, SEQ ID NO: 237, at least a second binding portion which binds to human CD38 comprises a CDR set selected from the group comprising SEQ ID NO: 103, SEQ ID NO: 163, SEQ ID NO: 223, and SEQ ID NO: 121; SEQ ID NO: 181, SEQ ID NO: 241; and at least one binding portion which binds to human CD47 comprising a CDR set comprising SEQ ID NO: 75, SEQ ID NO: 135, SEQ ID NO: 195.

In preferred aspects, the bispecific antibody for use disclosed herein further comprises a common light chain. More in particular, the bispecific antibody for use disclosed hereon further comprises a common light chain comprising an amino acid sequence of SEQ ID NO: 10. In another preferred aspects, the antibody for use disclosed herein further comprises a common light chain CDRs set comprising SEQ ID NO: 304, 305, and 306.

In certain embodiments of the present invention, the bispecific antibody is a full-length antibody, wherein the at least one binding portion which binds to human CD38 and/or the at least one of binding portion which binds to human CD47 is a Fab region. The term “Fab” or “Fab region” or “Fab domain” as used herein includes the polypeptides that comprise the VH, CH1, VL, and CL immunoglobulin domains. Fab may refer to this region in isolation, or this region in the context of a full-length antibody or antibody fragment. The present invention also provides an antibody fragment that binds to a human CD38 or to a human Cd47. Antibody fragments include, but are not limited to, (i) the Fab fragment consisting of VL, VH, CL and CH1 domains, including Fab′ and Fab′-SH, (ii) the Fd fragment consisting of the VH and CH1 domains, (iii) the Fv fragment consisting of the VL and VH domains of a single antibody; (iv) the dAb fragment (Ward E S et al., (1989) Nature, 341: 544-546) which consists of a single variable, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science 242: 423-426; Huston J S et al., (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion Tomlinson I & Hollinger P (2000) Methods Enzymol. 326: 461-79; WO94/13804; Holliger P et al., (1993) Proc. Natl. Acad. Sci. USA, 90: 6444-48) and (ix) scFv genetically fused to the same or a different antibody (Coloma MJ & Morrison SL (1997) Nature Biotechnology, 15(2): 159-163).

In certain embodiments the bispecific antibody of the present invention comprises at least one binding portion which binds to human CD47 and it is a Fab comprising and amino acid sequence selected from the group comprising SEQ ID NOs: 11, 12, 13, 14, 15, 16, 17, 18, 19, 20, 21, 299; in a particular embodiment the Fab which binds to human CD47 comprises a VH chain with a G65S mutation (Kabat numbering), in more particular embodiments the Fab which binds to human CD47 comprises an amino acid sequence of SEQ ID NO: 15 or 299. In a preferred embodiment, the bispecific antibody of the present invention comprises at least a Fab which binds to human CD47 having SEQ ID NO: 15 and comprising a VH chain with a G65S mutation (Kabat numbering), in particular the bispecific antibody of the present invention comprises at least a Fab which binds to human CD47 comprising an amino acid sequence of SEQ ID NO: 299. In certain embodiments the bispecific antibody of the present invention comprises at least one binding portion which binds to human CD38 and is a Fab comprising and amino acid sequence selected from the group comprising SEQ ID NOs: 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70. Preferably the amino acid sequence of the Fab which binds to human CD38 is selected from the group comprising SEQ ID NOs: 43, 52, 55, 57 and 61.

In particular embodiments of the present invention, the bispecific antibody is constructed using the BEAT® heavy chain (Hc) heterodimerization technology previously described (Skegro et al., (2017) J Biol Chem 292(23): 9745-9759 and Stutz et al., (2020) J Biol Chem 295(28): 9392-9408, WO2012131555), wherein the BEAT(A) chain, also referred herein to as BEAT(A), and the BEAT (B) chain, also referred herein to as BEAT(B). More specifically, the BEAT (A) Hc encompasses a VH domain with mutation G65S (Kabat numbering) a CH1 γ1 region, a γ1 hinge region, a γ1 CH2 region, and a γ3 based BEAT (A) CH3 domain assembled with a common light chain (cLc); in preferred embodiment the common light chain comprises an amino acid sequence of SEQ ID NO: 10. BEAT (B) Hc encompasses a VH domain, a CH1 γ1 region, a γ1 hinge region, a γ1 CH2 region, and a γ1 based BEAT (B) CH3 domain assembled with a cLc. According to the present invention the BEAT(A) heavy chain (Hc) comprises an amino acid sequence selected from the group comprising SEQ ID NOs: 259, 274, 276, 280, 281, 283, 285 and 278; the BEAT(B) Hc comprises an amino acid sequence selected from the group comprising SEQ ID NOs: 260, 261, 262, 275, 277, 279, 282, 284, 286, 287, 288, 289, 290, 291, 292 and 293. The cLc comprises an amino acid sequence of SEQ ID NO: 10. In preferred embodiments the bispecific antibody according to the present invention comprises a BEAT(A) chain comprising the amino acid sequence of SEQ ID NO: 259, a BEAT(B) chain comprising the amino acid sequence of SEQ ID NO: 260, and a cLc comprising the amino acid sequence of SEQ ID NO: 10; or a BEAT(A) chain comprising the amino acid sequence of SEQ ID NO: 259, a BEAT(B) chain comprising the amino acid sequence of SEQ ID NO: 261, and a cLc comprising the amino acid sequence of SEQ ID NO: 10; or a BEAT(A) chain comprising the amino acid sequence of SEQ ID NO: 259, a BEAT(B) chain comprising the amino acid sequence of SEQ ID NO: 262, and a cLc comprising the amino acid sequence of SEQ ID NO: 10; a BEAT(A) chain comprising the amino acid sequence of SEQ ID NO: 274, a BEAT(B) chain comprising the amino acid sequence of SEQ ID NO: 275, and a cLc comprising the amino acid sequence of SEQ ID NO: 10; or a BEAT(A) chain comprising the amino acid sequence of SEQ ID NO: 276, a BEAT(B) chain comprising the amino acid sequence of SEQ ID NO: 277, and a cLc comprising the amino acid sequence of SEQ ID NO: 10; or a BEAT(A) chain comprising the amino acid sequence of SEQ ID NO: 278, a BEAT(B) chain comprising the amino acid sequence of SEQ ID NO: 279, and a cLc comprising the amino acid sequence of SEQ ID NO: 10; or a BEAT(A) chain comprising the amino acid sequence of SEQ ID NO: 280, a BEAT(B) chain comprising the amino acid sequence of SEQ ID NO: 277, and a cLc comprising the amino acid sequence of SEQ ID NO: 10; or a BEAT(A) chain comprising the amino acid sequence of SEQ ID NO: 281, a BEAT(B) chain comprising the amino acid sequence of SEQ ID NO: 282, and a cLc comprising the amino acid sequence of SEQ ID NO: 10; or a BEAT(A) chain comprising the amino acid sequence of SEQ ID NO: 283, a BEAT(B) chain comprising the amino acid sequence of SEQ ID NO: 284, and a cLc comprising the amino acid sequence of SEQ ID NO: 10; or a BEAT(A) chain comprising the amino acid sequence of SEQ ID NO: 285, a BEAT(B) chain comprising the amino acid sequence of SEQ ID NO: 286, and a cLc comprising the amino acid sequence of SEQ ID NO: 10; or a BEAT(A) chain comprising the amino acid sequence of SEQ ID NO: 281, a BEAT(B) chain comprising the amino acid sequence of SEQ ID NO: 287, and a cLc comprising the amino acid sequence of SEQ ID NO: 10; or a BEAT(A) chain comprising the amino acid sequence of SEQ ID NO: 278, a BEAT(B) chain comprising the amino acid sequence of SEQ ID NO: 288, and a cLc comprising the amino acid sequence of SEQ ID NO: 10; or a BEAT(A) chain comprising the amino acid sequence of SEQ ID NO: 281, a BEAT(B) chain comprising the amino acid sequence of SEQ ID NO: 290, and a cLc comprising the amino acid sequence of SEQ ID NO: 10; or a BEAT(A) chain comprising the amino acid sequence of SEQ ID NO: 278, a BEAT(B) chain comprising the amino acid sequence of SEQ ID NO: 291, and a cLc comprising the amino acid sequence of SEQ ID NO: 10; or a BEAT(A) chain comprising the amino acid sequence of SEQ ID NO: 280, a BEAT(B) chain comprising the amino acid sequence of SEQ ID NO: 292, and a cLc comprising the amino acid sequence of SEQ ID NO: 10; or a BEAT(A) chain comprising the amino acid sequence of SEQ ID NO: 280, a BEAT(B) chain comprising the amino acid sequence of SEQ ID NO: 293, and a cLc comprising the amino acid sequence of SEQ ID NO: 10. In preferred embodiments, the bispecific antibody disclosed herein comprises a BAET(A) chain that binds to CD47 and comprises the amino acid sequence of SEQ ID NO: 280; a BEAT(B) chain that binds to CD38 and comprises an amino acid sequence of SEQ ID NOs: 292 or 293, more preferably the amino acid sequence of SEQ ID NO: 293; and a cLc comprising the amino acid sequence of SEQ ID NO: 10.

In certain aspects the present invention discloses a bispecific antibody comprising at least two binding portions, at least one of which binds to human CD38 and at least one of which binds to human CD47 wherein the at least one binding portion which binds to human CD47 has an affinity to human CD47 lower than the affinity that the at least one binding portion which binds to human CD38 has against human CD38. In particular, the at least one binding portion which binds to human CD47 has an affinity to human CD47 of about 1000 nM or less, or about 900 nM or less, or about 800 nM or less, or about 850 nM or less, or about 700 nM or less, or about 600 nM or less, or about 500 nM or less, or about 400 nM or less, or about 300 nM or less, 200 nM or less, or of about 150 nM or less, or of about 140 or less, or of about 130 nM or less, or of about 120 nM or less, preferably of about 110 nM or less, more preferably of 105 nM or less, for instance of about 104 nM; or preferably between about 700 nM and about 1000 nM, more preferably between about 798 nM and about 958 nM, more preferably about 878.3 nM. In particular, the at least one binding portion which binds to human CD38 has an affinity to human CD38 of about 100 nM or less, or of about 50 nM or less, or of about 30 or less, or of about 20 or less, or of about 10 or less, preferably of about 10 nM or less, of about 9 nM or less, of about 8 nM or less, of about 7 nM or less, of about 6 nM or less, of about 5 nM or less, of about 4 nM or less, of about 3 nM or less, of about 2 nM or less, preferably of about 1 nM or less, for instance of about 16.22 nM, about 17.6 nM, about 6.13 nM, about 2.3 nM, about 3.5 nM, about 0.55 nM, about 0.9 nM.

The present invention also discloses bispecific antibodies comprising at least one binding portion which binds to human CD47 and at least two binding portions which bind to human CD38, particular biparatopic bispecific antibodies wherein the affinity of the Fc proximal binding portion to a first epitope of human CD38 is about the same, or the same or different than the affinity of the Fc distal binding portion to a second epitope of human CD38.

### Nucleic Acids

Standard recombinant DNA methodologies are used to construct the polynucleotides that encode the polypeptides which form the binding proteins, incorporate these polynucleotides into recombinant expression vectors, and introduce such vectors into host cells. See e.g., Sambrook et al., 2001, MOLECULAR CLONING: A LABORATORY MANUAL (Cold Spring Harbor Laboratory Press, 3rd ed.). Enzymatic reactions and purification techniques may be performed according to manufacturer's specifications, as commonly accomplished in the art, or as described herein. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

Other aspects of the present disclosure relate to isolated nucleic acid molecules comprising a nucleotide sequence encoding any of the binding proteins described herein. In some embodiments, the isolated nucleic acid molecules comprise a sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical.

Certain aspects of the present disclosure relate to kits of polynucleotides. In some embodiments, one or more of the polynucleotides is a vector {e.g., an expression vector). The kits may find use, inter alia, in producing one or more of the binding proteins described herein, e.g., a bi-, or trispecific binding protein of the present disclosure. In some embodiments, the kit comprises one, two, three, or four polynucleotides.

In some embodiments, the isolated nucleic acid is operably linked to a heterologous promoter to direct transcription of the binding protein-coding nucleic acid sequence. A promoter may refer to nucleic acid control sequences which direct transcription of a nucleic acid. A first nucleic acid sequence is operably linked to a second nucleic acid sequence when the first nucleic acid sequence is placed in a functional relationship with the second nucleic acid sequence. For instance, a promoter is operably linked to a coding sequence of a binding protein if the promoter affects the transcription or expression of the coding sequence. Examples of promoters may include, but are not limited to, promoters obtained from the genomes of viruses (such as polyoma virus, fowlpox virus, adenovirus (such as Adenovirus 2), bovine papilloma virus, avian sarcoma virus, cytomegalovirus, a retrovirus, hepatitis-B virus, Simian Virus 40 (SV40), and the like), from heterologous eukaryotic promoters (such as the actin promoter, an immunoglobulin promoter, from heat-shock promoters, and the like), the CAG-promoter (Niwa et al., Gene 108(2): 193-9, 1991), the phosphogly cerate kinase (PGK)-promoter, a tetracycline-inducible promoter (Masui et al., Nucleic Acids Res. 33: e43, 2005), the lac system, the tip system, the tac system, the trc system, major operator and promoter regions of phage lambda, the promoter for 3-phosphoglycerate kinase, the promoters of yeast acid phosphatase, and the promoter of the yeast alpha-mating factors. Polynucleotides encoding binding proteins of the present disclosure may be under the control of a constitutive promoter, an inducible promoter, or any other suitable promoter described herein or other suitable promoter that will be readily recognized by one skilled in the art.

In some embodiments, the isolated nucleic acid is incorporated into a vector. In some embodiments, the vector is an expression vector. Expression vectors may include one or more regulatory sequences operatively linked to the polynucleotide to be expressed. The term “regulatory sequence” includes promoters, enhancers and other expression control elements (e.g., polyadenylation signals). Examples of suitable enhancers may include, but are not limited to, enhancer sequences from mammalian genes (such as globin, elastase, albumin, a-fetoprotein, insulin and the like), and enhancer sequences from a eukaryotic cell virus (such as SV40 enhancer on the late side of the replication origin (bp 100-270), the cytomegalovirus early promoter enhancer, the polyoma enhancer on the late side of the replication origin, adenovirus enhancers, and the like). Examples of suitable vectors may include, for example, plasmids, cosmids, episomes, transposons, and viral vectors (e.g., adenoviral, vaccinia viral, Sindbis-viral, measles, herpes viral, lentiviral, retroviral, adeno-associated viral vectors, etc.). Expression vectors can be used to transfect host cells, such as, for example, bacterial cells, yeast cells, insect cells, and mammalian cells. Biologically functional viral and plasmid DNA vectors capable of expression and replication in a host are known in the art and can be used to transfect any cell of interest.

Other aspects of the present disclosure relate to a vector system comprising one or more vectors encoding a first, second, third, and fourth polypeptide chain of any of the binding proteins described herein. In some embodiments, the vector system comprises a first vector encoding the first polypeptide chain of the binding protein, a second vector encoding the second polypeptide chain of the binding protein, a third vector encoding the third polypeptide chain of the binding protein, and a fourth vector encoding the fourth polypeptide chain of the binding protein. In some embodiments, the vector system comprises a first vector encoding the first and second polypeptide chains of the binding protein, and a second vector encoding the third and fourth polypeptide chains of the binding protein. In some embodiments, the vector system comprises a first vector encoding the first and third polypeptide chains of the binding protein, and a second vector encoding the second and fourth polypeptide chains of the binding protein. In some embodiments, the vector system comprises a first vector encoding the first and fourth polypeptide chains of the binding protein, and a second vector encoding the second and third polypeptide chains of the binding protein. In some embodiments, the vector system comprises a first vector encoding the first, second, third, and fourth polypeptide chains of the binding protein. The one or more vectors of the vector system may be any of the vectors described herein. In some embodiments, the one or more vectors are expression vectors.

### Isolated Host Cells

Other aspects of the present disclosure relate to an isolated host cell comprising one or more isolated polynucleotides, polynucleotide kits, vectors, and/or vector systems described herein. In some embodiments, the host cell is a bacterial cell (e.g., a E. coli cell). In some embodiments, the host cell is a yeast cell (e.g., an S. cerevisiae cell). In some embodiments, the host cell is an insect cell. Examples of insect host cells may include, for example, Drosophila cells (e.g., S2 cells), Trichoplusia ni cells (e.g., High Five™ cells), and Spodoptera frugiperda cells (e.g., Sf21 or Sf9 cells). In some embodiments, the host cell is a mammalian cell. Examples of mammalian host cells may include, for example, human embryonic kidney cells (e.g., 293 or 293 cells subcloned for growth in suspension culture), Expi293™ cells, CHO cells, baby hamster kidney cells (e.g., BHK, ATCC CCL 10), mouse Sertoli cells (e.g., TM4 cells), monkey kidney cells (e.g., CV1 ATCC CCL 70), African green monkey kidney cells (e.g., VERO-76, ATCC CRL-1587), human cervical carcinoma cells (e.g., HELA, ATCC CCL 2), canine kidney cells (e.g., MDCK, ATCC CCL 34), buffalo rat liver cells (e.g., BRL 3A, ATCC CRL 1442), human lung cells (e.g., W138, ATCC CCL 75), human liver cells (e.g., Hep G2, HB 8065), mouse mammary tumor cells (e.g., MMT 060562, ATCC CCL51), TRI cells, MRC 5 cells, FS4 cells, a human hepatoma line (e.g., Hep G2), and myeloma cells (e.g., NSO and Sp2/0 cells).

Other aspects of the present disclosure relate to a method of producing any of the binding proteins described herein. In some embodiments, the method includes a) culturing a host cell (e.g., any of the host cells described herein) comprising an isolated nucleic acid, vector, and/or vector system (e.g., any of the isolated nucleic acids, vectors, and/or vector systems described herein) under conditions such that the host cell expresses the binding protein; and b) isolating the binding protein from the host cell. Methods of culturing host cells under conditions to express a protein are well known to one of ordinary skill in the art. Methods of isolating proteins from cultured host cells are well known to one of ordinary skill in the art, including, for example, by affinity chromatography (e.g., two step affinity chromatography comprising protein A affinity chromatography followed by size exclusion chromatography).

In some embodiments, a binding protein of the present disclosure is purified by protein A affinity chromatography.

### Linkers

In some embodiments, the linkers L1, L2, L3 and L4 range from no amino acids (length=0) to about 100 amino acids long, or less than 100, 50, 40, 30, 20, or 15 amino acids or less. The linkers can also be 10, 9, 8, 7, 6, 5, 4, 3, 2, or 1 amino acids long. L1, L2, L3 and L4 in one binding protein may all have the same amino acid sequence or may all have different amino acid sequences.

Examples of suitable linkers include a single glycine (Gly) residue; a diglycine peptide (Gly-Gly); a tripeptide (Gly-Gly-Gly); a peptide with four glycine residues; a peptide with five glycine residues; a peptide with six glycine residues; a peptide with seven glycine residues; and a peptide with eight glycine residues. Other combinations of amino acid residues may be used such as the peptide GGGT (SEQ IS NO: 297), or the GGGGS, or repetitions of said peptides, such as the peptide GGGGS GGGGS GGGGS (SEQ ID NO: 298). In a preferred embodiment, the linker has an amino acid sequence of SEQ ID NO: 298.

The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins. For additional descriptions of linker sequences, see, e.g., WO2012135345 and International Application No. PCT/US2017/027488.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

### Fc Regions and Constant Domains

The present invention also relates to a bispecific antibody comprising an Fc region. In some embodiments, a binding protein of the present disclosure comprises an antibody fragment, including but not limited to antibody F(ab), F(ab′)2, Fab′-SH, Fv, or scFv fragments. In some embodiments, a binding protein of the present disclosure comprises an antibody fragment, including but not limited to antibody F(ab), F(ab′)2, Fab′-SH, Fv, or scFv fragments, comprising an Fc region.

In some embodiments, a binding protein of the present disclosure comprises a full-length antibody heavy chain or a polypeptide chain comprising an Fc region. In some embodiments, the Fc region is a human Fc region, e.g., a human IgG1, IgG2, IgG3, or IgG4 Fc region. In some embodiments, the Fc region includes an antibody hinge, CH1, CH2, CH3, and optionally CH4 domains. In some embodiments, the Fc region is a human IgG1 Fc region. In some embodiments, the Fc region is a human IgG4 Fc region. In some embodiments, the Fc region includes one or more of the mutations described herein.

In some embodiments, a binding protein of the present disclosure includes one or two Fc variants. The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “parent antibody” or “parent immunoglobulin” as used herein includes an unmodified antibody that is subsequently modified to generate a variant. Said parent antibody may be a naturally occurring antibody, a non-naturally occurring antibody, or a variant or engineered version of a naturally occurring antibody. Parent antibody may refer to the antibody itself, compositions that comprise the parent antibody, or the amino acid sequence that encodes it. In preferred embodiments of the present invention, the parent antibody comprises an Fc region. More specifically, the Fc region of the parent antibody according to the present invention is a human IgG1, IgG2, IgG3, or IgG4 Fc region; in some embodiments the Fc region of the parent antibody according to the present invention is a modified or not modified IgG1 Fc region.

In certain aspects, the bispecific antibody of the present invention comprises a variant Fc region which comprises at least one amino acid modification relative to the Fc region of the parent antibody, whereas the antibody comprising the variant Fc region exhibits altered effector function compared to the parent antibody. More specifically the CH2 domain of the Fc region comprises at least one amino acid modification.

In particular embodiments of the present invention, the bispecific antibody is constructed using the BEAT® heavy chain (Hc) heterodimerization technology previously described (Skegro et aL., (2017) J Biol Chem 292(23): 9745-9759 and Stutz et aL., (2020) J Biol Chem 295(28): 9392-9408, WO2012131555), wherein the BEAT(A) chain, also referred herein to as BEAT(A), and the BEAT (B) chain, also referred herein to as BEAT(B), have been engineered to as to increase the Fc effector function. More specifically the CH2 domain of the Fc region has been engineered so as to comprise at least one amino acid modification.

More specifically BEAT(A) comprises one or more substitutions at a position selected from the group comprising: 324, 334, 269, 298, 239, 332 and 333; and BEAT(B) comprises one or more substitutions at a position selected from the group comprising: 324, 334, 269, 298, 239, 332 and 333; preferably comprising 324, 334, 269, 289, 298, 333. Even more specifically BEAT(A) comprises one or more substitutions selected from the group comprising: S324N, K334E, K334A, E269D, S298A, S239D, 1332E and E333A; and BEAT(B) comprises one or more substitutions at a position selected from the group comprising: S324N, K334E, K334A, E269D, S289A, K334A, E333A. In certain particular embodiments, BEAT(A) comprises a set of mutations selected from the group comprising: S324N; or S324N and K334E; or E269D, S298A, S324N and K334A; or S239D, 1332E and S324N; or E269D, S298A, S324N and E334A; or S298A, S324N and E333A; or S298A, S324N and K334A; or S324N, S298A, E269D and E333A; or S324N, S298A, E269D and K334A. In other particular embodiments, BEAT(B) comprises a set of mutations selected from the group comprising: S324N; or S324N and K334E; or E269D, S298A, S324N and K334A; or S239D, 1332E and S324N; or E269D; or E269D, S298A, S324N and E334A; or S298A, S324N and E333A; or S298A, S324N and K334A; or S324N, S298A, E269D and E333A; or S324N, S298A, E269D and K334A. In certain preferred embodiments, BEAT(A) and BEAT(B) comprises the mutation S324N; or the mutations S324N and K334E; or the mutations E269D, S298A, S324N and K334A; or the mutations E269D, S298A, S324N and E334A; or the mutations S298A, S324N and E333A; or the mutations S298A, S324N and K334A; or the mutations S324N and K334E. In particularly preferred embodiments, BEAT(A) comprises the mutations S239D, 1332E and S324N, and BEAT(B) comprises the mutation S324N.

In some embodiments, the Fc region comprises one or more mutations that reduce or eliminate Fc receptor binding and/or effector function of the Fc region (e.g., Fc receptor-mediated antibody-dependent cellular phagocytosis (ADCP), complement-dependent cytotoxicity (CDC), and/or antibody-dependent cellular cytotoxicity (ADCC)).

In some embodiments, the Fc region is a human IgG1 Fc region comprising one or more amino acid substitutions at positions corresponding to positions 234, 235, and/or 329 of human IgG1 according to EU Index. In some embodiments, the amino acid substitutions are L234A, L235A, and/or P329A. In some embodiments, the Fc region is a human IgG1 Fc region comprising amino acid substitutions at positions corresponding to positions 298, 299, and/or 300 of human IgG1 according to EU Index. In some embodiments, the amino acid substitutions are S298N, T299A, and/or Y300S.

In some embodiments, the Fc region is a human IgG4 Fc region comprising one or more mutations that reduce or eliminate FcγI and/or FcγII binding. In some embodiments, the Fc region is a human IgG4 Fc region comprising one or more mutations that reduce or eliminate FcγI and/or FcγII binding but do not affect FcRn binding. In some embodiments, the Fc region is a human IgG4 Fc region comprising amino acid substitutions at positions corresponding to positions 228 and/or 409 of human IgG4 according to EU Index. In some embodiments, the amino acid substitutions are S228P and/or R409K. In some embodiments, the Fc region is a human IgG4 Fc region comprising amino acid substitutions at positions corresponding to positions 234 and/or 235 of human IgG4 according to EU Index. In some embodiments, the amino acid substitutions are F234A and/or L235A. In some embodiments, the Fc region is a human IgG4 Fc region comprising amino acid substitutions at positions corresponding to positions 228, 234, 235, and/or 409 of human IgG4 according to EU Index. In some embodiments, the amino acid substitutions are S228P, F234A, L235A, and/or R409K. In some embodiments, the Fc region is a human IgG4 Fc region comprising amino acid substitutions at positions corresponding to positions 233-236 of human IgG4 according to EU Index. In some embodiments, the amino acid substitutions are E233P, F234V, L235A, and a deletion at 236. In some embodiments, the Fc region is a human IgG4 Fc region comprising amino acid mutations at substitutions corresponding to positions 228, 233-236, and/or 409 of human IgG4 according to EU Index. In some embodiments, the amino acid mutations are S228P; E233P, F234V, L235A, and a deletion at 236; and/or R409K.

In some embodiments, a binding protein of the present disclosure comprises one or more mutations to improve purification, e.g., by modulating the affinity for a purification reagent. For example, it is known that heterodimeric binding proteins can be selectively purified away from their homodimeric forms if one of the two Fc regions of the heterodimeric form contains mutation(s) that reduce or eliminate binding to Protein A, because the heterodimeric form will have an intermediate affinity for Protein A-based purification than either homodimeric form and can be selectively eluted from Protein A, e.g., by use of a different pH (See e.g., Smith, E. J. et al. (2015) Sci. Rep. 5: 17943). In some embodiments, the mutation comprises substitutions at positions corresponding to positions 435 and 436 of human IgG1 or IgG4 according to EU Index, wherein the amino acid substitutions are H435R and Y436F. In some embodiments, the binding protein comprises a second polypeptide chain further comprising a first Fc region linked to CH1, the first Fc region comprising an immunoglobulin hinge region and CH2 and CH3 immunoglobulin heavy chain constant domains, and a third polypeptide chain further comprising a second Fc region linked to CH1, the second Fc region comprising an immunoglobulin hinge region CH2 and CH3 immunoglobulin heavy chain constant domains; and wherein only one of the first and the second Fc regions comprises amino acid substitutions at positions corresponding to positions 435 and 436 of human IgG1 or IgG4 according to EU Index, wherein the amino acid substitutions are H435R and Y436F. In some embodiments, a binding protein of the present disclosure comprises knob and hole mutations and one or more mutations to improve purification. In some embodiments, the first and/or second Fc regions are human IgG1 Fc regions. In some embodiments, the first and/or second Fc regions are human IgG4 Fc regions.

To improve the yields of some binding proteins (e.g., bispecific or trispecific binding proteins), the CH domains can be altered by the BEAT technology which is described in detail with several examples in and in International Publication No. WO2012131555.

In some embodiments, a binding protein of the present disclosure comprises one or more mutations to improve serum half-life (See e.g., Hinton, P R. et al. (2006) J. Immunol. 176(1):346-56). In some embodiments, the mutation comprises substitutions at positions corresponding to positions 428 and 434 of human IgG1 or IgG4 according to EU Index, wherein the amino acid substitutions are M428L and N434S.

In some embodiments, the binding protein comprises a second polypeptide chain further comprising a first Fc region linked to CH1, the first Fc region comprising an immunoglobulin hinge region and CH2 and CH3 immunoglobulin heavy chain constant domains, and a third polypeptide chain further comprising a second Fc region linked to CH1, the second Fc region comprising an immunoglobulin hinge region and CH2 and CH3 immunoglobulin heavy chain constant domains, wherein the first and/or second Fc regions comprise amino acid substitutions at positions corresponding to positions 428 and 434 of human IgG1 or IgG4 according to EU Index, wherein the amino acid substitutions are M428L and N434S. In some embodiments, a binding protein of the present disclosure comprises knob and hole mutations and one or more mutations to improve serum half-life. In some embodiments, the first and/or second Fc regions are human IgG1 Fc regions. In some embodiments, the first and/or second Fc regions are human IgG4 Fc regions.

In some embodiments, a binding protein of the present disclosure comprises one or more mutations to reduce effector function, e.g., Fc receptor-mediated antibody-dependent cellular phagocytosis (ADCP), complement-dependent cytotoxicity (CDC), and/or antibody-dependent cellular cytotoxicity (ADCC). In some embodiments, the second polypeptide chain further comprises a first Fc region linked to CH1, the first Fc region comprising an immunoglobulin hinge region and CH2 and CH3 immunoglobulin heavy chain constant domains; wherein the third polypeptide chain further comprises a second Fc region linked to CH1, the second Fc region comprising an immunoglobulin hinge region and CH2 and CH3 immunoglobulin heavy chain constant domains; wherein the first and second Fc regions are human IgG1 Fc regions; and wherein the first and the second Fc regions each comprise amino acid substitutions at positions corresponding to positions 234 and 235 of human IgG1 according to EU Index, wherein the amino acid substitutions are L234A and L235A. In some embodiments, the Fc regions of the second and the third polypeptide chains are human IgG1 Fc regions, and wherein the Fc regions each comprise amino acid substitutions at positions corresponding to positions 234 and 235 of human IgG1 according to EU Index, wherein the amino acid substitutions are L234A and L235A. In some embodiments, the second polypeptide chain further comprises a first Fc region linked to CH1, the first Fc region comprising an immunoglobulin hinge region and CH2 and CH3 immunoglobulin heavy chain constant domains; wherein the third polypeptide chain further comprises a second Fc region linked to CH1, the second Fc region comprising an immunoglobulin hinge region and CH2 and CH3 immunoglobulin heavy chain constant domains; wherein the first and second Fc regions are human IgG1 Fc regions; and wherein the first and the second Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG1 Fc regions, and wherein the Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG4 Fc regions, and the Fc regions each comprise amino acid substitutions at positions corresponding to positions 234 and 235 of human IgG4 according to EU Index, wherein the amino acid substitutions are F234A and L235A. In some embodiments, the binding protein comprises a second polypeptide chain further comprising a first Fc region linked to CH1, the first Fc region comprising an immunoglobulin hinge region and CH2 and CH3 immunoglobulin heavy chain constant domains, and a third polypeptide chain further comprising a second Fc region linked to CH1, the second Fc region comprising an immunoglobulin hinge region and CH2 and CH3 immunoglobulin heavy chain constant domains; and wherein the first and the second Fc regions each comprise amino acid substitutions at positions corresponding to positions 234 and 235 of human IgG4 according to EU Index, wherein the amino acid substitutions are F234A and L235A.

In some embodiments, a binding protein of the present disclosure comprises knob and hole mutations and one or more mutations to reduce effector function. In some embodiments, the first and/or second Fc regions are human IgG1 Fc regions. In some embodiments, the first and/or second Fc regions are human IgG4 Fc regions. For further description of Fc mutations at position 329, see, e.g., Shields, R. L. et al. (2001) J. Biol. Chem. 276:6591-6604 and WO 1999051642.

### Application of the Binding Protein

The binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in certain embodiments, binding proteins can be labeled with a detectable moiety. The detectable moiety can be any one that is capable of producing, either directly or indirectly, a detectable signal. For example, the detectable moiety can be a radioisotope, such as 3H,14C,32P,35S,1251,99Tc,U11n, or67Ga; a fluorescent or chemiluminescent compound, such as fluorescein isothiocyanate, rhodamine, or luciferin; or an enzyme, such as alkaline phosphatase, 3-galactosidase, or horseradish peroxidase.

The binding proteins are also useful for in vivo imaging. A binding protein labeled with a detectable moiety can be administered to an animal, preferably into the bloodstream, and the presence and location of the labeled antibody in the host assayed. The binding protein can be labeled with any moiety that is detectable in an animal, whether by nuclear magnetic resonance, radiology, or other detection means known in the art.

For clinical or research applications, in certain embodiments, binding proteins can be conjugated to a cytotoxic agent. A variety of antibodies coupled to cytotoxic agents {i.e., antibody-drug conjugates) have been used to target cytotoxic payloads to specific tumor cells. Cytotoxic agents and linkers that conjugate the agents to an antibody are known in the art; see, e.g., Parslow, A. C. et al. (2016) Biomedicines 4: 14 and Kalim, M. et al. (2017) Drug Des. Devel. Ther. 11: 2265-2276.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper pierceable by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, a binding protein of the present disclosure is administered to a patient in need thereof for the treatment or prevention of cancer. In some embodiments, the present disclosure relates to a method of preventing and/or treating a proliferative disease or disorder (e.g., cancer). In some embodiments, the method comprises administering to a patient a therapeutically effective amount of the heterodimeric antibody, or pharmaceutical compositions related thereto, described herein. In some embodiments, the present disclosure relates to uses of the heterodimeric antibody, or pharmaceutical compositions related thereto, described herein for preventing and/or treating a proliferative disease or disorder (e.g., cancer) in a patient in need thereof. In some embodiments, the present disclosure relates to the heterodimeric antibody, or pharmaceutical compositions related thereto, described herein for use in the manufacture of a medicament for preventing and/or treating a proliferative disease or disorder (e.g., cancer) in a patient in need thereof. In some embodiments, the patient is a human. In some embodiments, the binding protein comprises one antigen binding site that binds a T-cell surface protein and another antigen binding site that binds the extracellular domain of a human CD38 polypeptide. In some embodiments, the binding protein comprises an antigen binding site that binds the extracellular domain of a human CD38 polypeptide and an antigen binding site that binds a human CD47 polypeptide.

In some embodiments, cells of the cancer express a human CD38 1 polypeptide on their cell surface (e.g., comprising the amino acid sequence of SEQ ID NO: 1). In some embodiments, cells of the cancer express a human CD38 isoform SEQ ID NO: 295 (human CD38 isoform 28907-1), SEQ ID NO: 294 (human CD38 isoform 28907-2), SEQ ID NO: 296 (human CD38 isoform 28907-E). In some embodiments, the patient is selected for treatment on the basis that the cells of the cancer express a human CD38 isoform SEQ ID NO: 295 (human CD38 isoform 28907-1), SEQ ID NO: 294 (human CD38 isoform 28907-2), SEQ ID NO: 296 (human CD38 isoform 28907-E).

In certain embodiments, the cancer is selected from the group comprising: for use in treating multiple myeloma, acute lymphoblastic leukemia, chronic lymphocytic leukemia, acute myeloid leukemia, lymphoma, breast cancer such as Her2+ breast cancer, prostate cancer, cervival cancer, germinal center B-cell lympohoma or B-cell acute lymphoblastic leukemia, Chronic lymphocytic leukemia (CLL), Myelodisplastic syndrome (MDS), Non-Hodgkin lymphoma, diffuse large B-cell lymphoma, non small cell lung cancer (NSCLC), Hepatocellular carcinoma (HCC), High-grade serous ovarian carcinoma, peritoneal cancer. In a specific embodiment the cancer is multiple myeloma. Anti-CD38 antibodies have been tested for the treatment of multiple myeloma, such as Daratumumab, also called herein Darzalex® and isatuximab. However, while multiple myeloma is considered treatable, relapse is inevitable in almost all patients, leading to the development of treatment-refractory disease. In some embodiments, the cancer is relapsed or refractory multiple myeloma. In some embodiments, the patient has been treated with a prior multiple myeloma treatment. In some embodiments, a binding protein of the present disclosure is administered to the patient as a 1st, 2nd, or 3rdline treatment for multiple myeloma.

In certain embodiments, the bispecific antibody according to the present invention which has at least one binding portion that binds to CD47 allows blocking the interaction between CD47 and SIRPα, avoiding the transmission of a “Don't eat me” signal due to CD47-SIRPα interaction, which inhibits macrophages phagocytosis. An exemplary clinical benchmark monoclonal antibody which binds to CD47 is Magrolimab, also called herein 5F9. In the present invention the terms “Magrolimab” and “5F9” are used interchangeably. In certain embodiments the tested molecule is a “59F-like” (also called herein “Magrolimab-like”) antibody which is the same as the clinical Magrolimab molecule but it is produced in house. In the present invention the terms “Magrolimab”, “5F9”, “59F-like” and “Magrolimab-like” are interchangeable. More in particular the bispecific antibody comprising at least two binding portions, at least one of which binds to human CD38 and at least one of which binds to human CD47, blocks the CD47-SIRPα interaction and therefore the “Don't eat me” signal selectively in CD38 expressing cells.

In certain embodiments, the bispecific antibody according to the present invention shows higher binding to CD38 high tumor cell lines (such as Daudi, Raji, KMS-12-PE) as compared to benchmark Darzalex and 5F9, in vitro. In other embodiments the bispecific antibody according to the present invention shows higher binding to CD38 low tumor cell lines (NCI-H929 and KMS-12-BM) as compared to Darzalex. In certain embodiments, the bispecific antibodies according to the present invention show a comparable efficiency as that of 5F9 at inducing phagocytosis of both CD38^{high} or CD38^{low} expressing tumor cells, in vitro. In certain embodiments, the bispecific antibodies according to the present invention show higher tumor cells killing by CDC as compared to Darzalex, in vitro. In certain embodiments, the bispecific antibodies according to the present invention show comparable killing by ADCC of CD^{high} or CD38^{low} expressing tumor cells as that induced by Darzalex and on CD38^{low} expressing tumor cells higher potency, in vitro. In certain embodiments, the bispecific antibodies according to the present invention show higher potency as compared to the combination of Darzalex and 5F9, in vitro.

### Binding Protein Therapeutic Compositions and Administration Thereof

Therapeutic or pharmaceutical compositions comprising binding proteins are within the scope of the disclosure. Such therapeutic or pharmaceutical compositions can comprise a therapeutically effective amount of a binding protein, or binding protein-drug conjugate, in admixture with a pharmaceutically or physiologically acceptable formulation agent selected for suitability with the mode of administration. Acceptable formulation materials preferably are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles.

Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery or subcutaneous. Alternatively, the compositions can be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polygly colic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

In one embodiment, a pharmaceutical composition can be formulated for inhalation. For example, a binding protein can be formulated as a dry powder for inhalation. Binding protein inhalation solutions can also be formulated with a propellant for aerosol delivery. In yet another embodiment, solutions can be nebulized.

It is also contemplated that certain formulations can be administered orally. In one embodiment of the disclosure, binding proteins that are administered in this fashion can be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents can be included to facilitate absorption of the binding protein. Diluents, flavorings, low melting point waxes, vegetable oils, lubricants, suspending agents, tablet disintegrating agents, and binders can also be employed.

Another pharmaceutical composition can involve an effective quantity of binding proteins in a mixture with non-toxic excipients that are suitable for the manufacture of tablets. By dissolving the tablets in sterile water, or another appropriate vehicle, solutions can be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(−)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

The disclosure also encompasses kits for producing a single-dose administration unit. The kits can each contain both a first container having a dried protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

The disclosure also provides a method of treating multiple myeloma, AML and/or T-ALL in a subject in need thereof. The method employs a heterodimeric antibody that co-engages CD47 and CD38 in such a manner so as to transiently connect malignant cells with T cells, thereby inducing T cell mediated killing of the bound malignant cell. The method described herein utilizes a heterodimeric antibody that binds CD47 and CD38 in such a manner so as to maximize destruction of target cells while reducing unwanted side effects (e.g., uncontrolled cytokine release).

In particular the present disclosure provides a bispecific antibody comprising at least one binding portion which binds to human CD47 and at least two binding portions which bind to human CD38, for use in the treatment of multiple myeloma (MM), relapsed refractory multiple myeloma (RRMM), Acute myeloid leukemia (AML) orT-cell acute lymphoblastic leukemia (T-ALL).

The present disclosure also provides a method of treatment multiple myeloma (MM), relapsed refractory multiple myeloma (RRMM), Acute myeloid leukemia (AML) orT-cell acute lymphoblastic leukemia (T-ALL). using a bispecific antibody comprising at least one binding portion which binds to human CD47 and at least two binding portions which bind to human CD38.

The present invention also relates to the use of the bispecific antibody comprising at least one binding portion which binds to human CD47 and at least two binding portions which bind to human CD38 disclosed inhere to treat multiple myeloma (MM), relapsed refractory multiple myeloma (RRMM), Acute myeloid leukemia (AML) orT-cell acute lymphoblastic leukemia (T-ALL).

### Therapeutic Regimen

The heterodimeric antibody is administered to a subject in need thereof, e.g., a human subject suffering from multiple myeloma, such as relapsed/refractory multiple myeloma, acute myeloid leukemia (AML) or T-cell acute lymphoblastic leukemia. Relapsed myeloma is characterized as a recurrence of disease after prior response. Examples of laboratory and radiological criteria signaling the disease include, but are not limited to, >25% increase of the serum or urine monoclonal protein (M-protein) or >25% difference between involved and uninvolved serum free light chains from nadir, respectively, or the development of new plasmacytomas or hypercalciemia. Sonneveld et al., Haematologica. 2016 April; 101(4): 396-406.

In non-secretory disease patients, relapse is characterized by an increase of the bone marrow plasma cells. A signal for relapsed disease also is characterized by the appearance or reappearance of one or more CRAB criteria or a rapid and consistent biochemical relapse. Refractory myeloma is myeloma that is not responsive to treatment. Relapsed/refractory multiple myeloma refers to the disease which becomes non-responsive or progressive on therapy or within 60 days of the last treatment in patients who previously achieved at least a minimal response on previous therapy. Sonneveld, supra; Anderson et al., Leukemia. 2008; 22(2):231-239.

The method of the disclosure comprises administering to the subject a dose of about 0.05 mg to about 200 mg of the heterodimeric antibody. The dose is, in various embodiments, about 0.5 mg to about 200 mg, about 0.5 to about 150 mg, about 1 mg to about 150 mg, about 10 mg to about 100 mg, about 10 mg to about 200 mg, about 4 mg to about 200 mg, about 12 mg to about 200 mg, about 12 mg to about 100 mg, about 36 mg to about 200 mg, about 36 mg to about 100 mg, or about 100 mg to about 200 mg. In various aspects of the method, the dose administered to the subject is about 0.05 mg, about 0.15 mg, about 0.45 mg, about 1.35 mg, about 4 mg, about 12 mg, about 36 mg, about 100 mg, or about 200 mg.

In alternative aspects, a single dose of heterodimeric antibody is at least about 0.05 mg, at least about 0.15 mg, at least about 0.45 mg, at least about 1.35 mg, at least about 4 mg, at least about 12 mg, at least about 36 mg, or at least about 100 mg. In various aspects, a single dose of heterodimeric antibody is no more than about 200 mg (e.g., no more than about 100 mg or no more than about 36 mg). It will be appreciated that a single dose may be administered via multiple administrations (i.e., a divided dose), such that the multiple administrations combine to the dose recited herein. For example, multiple administrations (e.g., two or more injections) combine to be at least about 0.05 mg, at least about 0.15 mg, at least about 0.45 mg, at least about 1.35 mg, at least about 4 mg, at least about 12 mg, at least about 36 mg, or at least about 100 mg. In various aspects, the multiple administrations of a single dose of heterodimeric antibody combine to be no more than about 200 mg (e.g., no more than about 100 mg or no more than about 36 mg).

In various aspects of the method, the dose is adjusted over the course of treatment. For example, the subject is administered an initial dose at one or more administrations, and a higher dose is used in one or more subsequent administrations. Put another way, the disclosure contemplates increasing the dose of heterodimeric antibody at least once over the course of treatment. Alternatively, the dose may be decreased over the course of treatment, such that amount of heterodimeric antibody is reduced as treatment progresses.

The disclosure contemplates a method wherein multiple (i.e., two or more) doses of the heterodimeric antibody are administered over the course of a treatment period. The individual doses may be administered at any interval, such as once a week, twice a week, three times a week, four times a week, or five times a week. Individual doses may be administered every two weeks, every three weeks, or every four weeks. In other words, in some aspects, a waiting period of at two weeks passes between heterodimeric antibody administrations to the subject. The waiting period between administrations of the doses need not be consistent over the course of the treatment period. In other words, the interval between doses can be adjusted over the course of treatment. In some aspects, the method comprises administering two doses of heterodimeric antibody per week to the subject in the first and second weeks of treatment (i.e., twice a week for weeks 1 and 2), administering one dose of heterodimeric antibody per week to the subject in the third and fourth weeks of treatment (i.e., once a week for weeks 3 and 4), and administering one dose of heterodimeric antibody every two weeks starting in week 5 through the end of treatment (i.e., there is a waiting period of two weeks between doses starting in week 5 through the end of treatment). While not wishing to be bound to any particular theory, the shorter interval between doses for the first administrations (e.g., two doses per week) promotes rapid target cell clearance. Increasing the interval between doses as set forth herein maintains cell clearance while minimizing unwanted side effects associated with immunotherapy.

Alternatively, in various aspects, the method comprises administering one dose of heterodimeric antibody per week for weeks 1-4 of treatment, and optionally administering one dose of the heterodimeric antibody every two weeks starting in week 5 through the end of treatment.

The multiple doses of heterodimeric antibody are administered over treatment period of, e.g., three months to about 18 months, or about three months to about 12 months, or about three months to about nine months, or about three months to about six months, or about three months to about eight months, or about six months to about 18 months, or about six months to about 12 months, or about eight months to about 12 months, or about six months to about eight months, or about eight months to about 12 months (e.g., about eight months). Optionally, the multiple (i.e., two or more) doses of the heterodimeric antibody are administered over a treatment period of about 12 weeks to about 52 weeks, or about 12 weeks to about 36 weeks, or about 24 weeks to about 32 weeks, with doses administered twice a week, once a week, once every two weeks, or once every four weeks.

By “treating” multiple myeloma is meant achievement of any positive therapeutic response with respect to the disease. For example, a positive therapeutic response includes one or more of the following improvements in the disease: (1) a reduction in the number of neoplastic cells; (2) an increase in neoplastic cell death; (3) inhibition of neoplastic cell survival; (4) reduction in paraprotein production by tumor cells; (5) inhibition (i.e., slowing to some extent, preferably halting) of tumor growth; (6) an increased patient survival rate; and (7) some relief from one or more symptoms associated with the disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation. A complete therapeutic response (i.e., absence of clinically detectable disease with normalization of any previously abnormal radiographic studies, bone marrow, and cerebrospinal fluid (CSF) or abnormal monoclonal protein) is not required; any degree of improvement is contemplated. Various additional parameters associated with disease treatment and improvement are set forth in the Examples.

The heterodimeric antibody may be administered via any suitable means to the subject, e.g., via intravenous, intraarterial, intralymphatic, intrathecal, intracerebral, intraperitoneal, intracerobrospinal, intradermal, subcutaneous, intraarticular, intrasynovial, oral, topical, or inhalation routes. For example, the heterodimeric antibody may be administered via intravenous administration as a bolus or by continuous infusion over a period of time. In various aspects, the method comprises administering the heterodimeric antibody via intravenous infusion over a period of about 30 minutes to about four hours. Optionally, the time for infusion is decreased in subsequent administrations. For example, in one embodiment, the first dose of heterodimeric antibody is administered over a period of about four hours, and subsequent doses are administered over a period of two hours or less. In this regard, the first dose of heterodimeric antibody is optionally administered over a period of about four hours, the second dose of heterodimeric antibody is optionally administered over a period of about two hours, and subsequent doses are optionally administered over a period of about 30 minutes.

In some instances, the subject has previously been treated for multiple myeloma. For example, the subject may have previously been administered an immunomodulatory drug (thalidomide, lenalidomide, pomalidomide), a proteasome inhibitor (such as pomalidomide, bortezomib, or carfilzomib), dexamethasone, doxorubicin, or combinations thereof.

Optionally, the subject was previously treated with an anti-CD38 monospecific antibody, such as daratumumab (DARZALEX®). In various embodiments, the subject is relapsed or refractory with prior anti-CD38 monospecific antibody treatment. When the patient has been treated with anti-CD38 monospecific antibody, the initial dose of the heterodimeric antibody is preferably administered following a wash-out period sufficient to reduce systemic concentration of the anti-CD38 monospecific antibody to 0.2 pg/ml or less. Put another way, the method comprises a waiting period between the previous administration of anti-CD38 monospecific antibody and administration of the heterodimeric antibody. In various embodiments, the method comprises ceasing treatment with the anti-CD38 monospecific antibody for at least 12 weeks (e.g., about 13 to about 15 weeks) prior to administering an initial dose of the heterodimeric antibody.

### Co-Therapy

Optionally, the heterodimeric antibody is part of a therapeutic regimen that comprises administration of one or more other therapeutic agents, radiation therapy, stem cell transplantation, and the like.

The method of the disclosure optionally further comprises administering dexamethasone to the subject. The dexamethasone may be administered by any route, such as the routes described here. Preferably, the dexamethasone is administered intravenously or orally. When the dexamethasone is administered intravenously, it is optionally administered to the subject within one hour prior to administration of the heterodimeric antibody. The dexamethasone is optionally administered in an amount of about 8 mg or about 4 mg.

In various embodiments, the method of the disclosure further comprises administering a chemotherapeutic agent. Non-limiting examples of DNA damaging chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea).

Chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib, CEP-18770, MG132, peptide vinyl sulfones, peptide epoxyketones (such as epoxomicin and carfilzomib), beta-lactone inhibitors (such as lactacystin, MLN 519, NPI-0052, Salinosporamide A), compounds that create dithiocarbamate complexes with metals (such as Disulfiram), and certain antioxidants (such as Epigallocatechin-3-gallate, catechin-3-gallate, and Salinosporamide A); NF-kB inhibitors, including inhibitors of IKB kinase; antibodies which bind to proteins overexpressed in cancers and thereby downregulate cell replication (e.g., trastuzumab, rituximab, cetuximab, and bevacizumab); and other inhibitors of proteins or enzymes known to be upregulated, over-expressed or activated in cancers, the inhibition of which downregulates cell replication.

The therapeutic regimen may comprise administration of other antibody therapeutics, such as elotuzumab (a humanized monoclonal against SLAMF7; Tai et al., Blood, 2008; 112:1329-37); daratumumab, MOR202, and isatuximab that target CD38; nBT062-SMCC-DMI, nBT062-SPDB-DM4, and nBT062-SPP-DMI that target CD138; lucatumumab (also known as HCD122) and dacetuzumab (also known as SGN-40) that target CD40; Lorvotuzumab which targets CD56. For a review of antibody therapeutics for the treatment of multiple myeloma, see, e.g., Tandon et al., Oncology & Hematology Review, 2015; 11(2):115-21, and Sondergeld et al., Clinical Advances in Hematology & Oncology, 2015; 13(9), 599, both incorporated by reference.

In some embodiments, the heterodimeric antibody is administered prior to, concurrent with, or after treatment with Velcade® (bortezomib), Thalomid™ (thalidomide), Aredia™ (pamidronate), or Zometa™ (zoledronic acid).

All cited references are herein expressly incorporated by reference in their entirety. Whereas particular embodiments of the invention have been described above for purposes of illustration, it will be appreciated by those skilled in the art that numerous variations of the details may be made without departing from the invention as described in the appended claims.

### FIGURES

FIG. 1. Cumulative analyses of Maximum phagocytosis of ADCP assay performed with CD38int (MOLM-13, left panel) or CD38low (KG-1, right panel) AML tumor cell lines. Mean of n=6-8 experiments+/−SD is represented. BEAT CD38/47 induces higher phagocytosis of AML cell lines with intermediate or low CD38 expression as compared to daratumumab.

FIG. 2. Cumulative analyses of ADCC (A) or MMoAK (B) assays performed with CD38int (MOLM-13) AML tumor cell line. Mean of n=6-8 experiments+/−SD is represented. BEAT CD38/47 shows higher potency than daratumumab in ADCC and induces higher killing of AML cell line in MMoAK assays as compared to anti-CD47 (5F9).

FIG. 3. Cumulative analyses of Maximum phagocytosis of ADCP assay (A) performed with CD38ihigh (ALL-SIL) or CD38low (PEER) T-ALL tumor cell lines. ADCC assay (B) performed with CD38low (PEER) T-ALL tumor cell lines. Mean of n=6-8 experiments+/−SD is represented. BEAT CD38/47 induces higher killing of T-ALL cell lines with intermediate or low CD38 expression as compared to daratumumab as measured by ADCC or phagocytosis.

FIG. 4. (A-B) Tumor killing assay performed on ex vivo AML bone marrow aspirates. (A) BEAT CD38/47 induces detectable killing of AML blast in 5 out of 12 samples tested using autologous killing assay of patients' samples ex vivo. (B) A higher trend in efficacy was mediated by BEAT CD38/47 in 5 out of the 6 samples where killing was detectable ex vivo as compared to isotype control and benchmarks.

FIG. 5. (A) Absolute quantification of CD38 by sABC on AML cell lines and on CD34+CD45low cells from AML patient samples. (B) Absolute quantification of CD38 by sABC on CD34+CD45low cells from 38 AML patient samples. (C) Compiled analysis of AML blast cell killing induced by BEAT CD38/47 treatment used at 100 nM as compared to isotype control used at 100 nM. (n=20 bone-marrow samples from AML patients; only samples for which killing was detected were included). Stats: Paired T-Test. *p<0.05. (D) Maximal Phagocytosis induced by BEAT CD38/47 as compared to isotype control antibody on 11 AML patient samples. Stats: Paired T-Test. ***p<0.001.

### EXAMPLE 1: BEAT CD38/47 INDUCES HIGHER PHAGOCYTOSIS OF AML CELL LINES WITH INTERMEDIATE (CD38INT) OR LOW CD38 (CD38LOW) EXPRESSION AS COMPARED TO DARATUMUMAB

### Material and Methods

The potential of BEAT CD38/47 to induce phagocytosis of AML cell lines has been measured with an Antibody-Dependent-Cellular Phagocytosis (ADCP) assay using the pH-rodo readout, using human Monocyte-Derived-Macrophages as effector cells and pH-rodo-labelled tumor cell lines as target cells, in a 1/3 E/T ratio.

### Monocyte Isolation and Differentiation in MO-MDM

7 days before the assay, Peripheral Blood Mononuclear Cells (PMBCs) from Healthy Volunteers were harvested from buffy coats obtained from Bern Transfusion Center. Buffy coats were diluted 1/2 in sterile PBS and then transferred in 3 SepMate tubes containing 12 ml Ficoll. Isolation was performed following manufacturer's instructions. The mononuclear layer was transferred into 50 ml falcon tubes. PBMCs were washed 3 times in PBS (300 g for 10 min), counted and finally suspended at a concentration of 50×106 cells/ml in Easysep Buffer for subsequent monocyte isolation. Human monocytes were isolated from PBMC using EasySep Monocytes Isolation kit according to the manufacturer's instructions. Isolated monocytes were then differentiated into Monocyte-Derived-Macrophages by culturing them in complete medium containing 50 ng/mL human M-CSF for 7 days at a density of 0.5×106 cells/ml.

### Antibody Dependent Cellular Phagocytosis Assay

On the day of the experiment, Monocyte-Derived-Macrophages were detached using Cell-Dissociation-Buffer, washed with PBS and then labelled with 2.5 μM Cell trace violet. Tumor cells were washed with PBS and labelled with 0.5 pg/mL pH-rodo Red. 9×103 pH-rodo-labelled tumor cells were then plated with 3×103 Cell-Trace Violet-labeled monocytes-derived-macrophages (effector: target ratio=1:3) in each well of a 384-well clear-bottom plate. Increasing concentration of test antibody was subsequently added to the well and incubated for 1h30 at 37 °C, 5% C02. Phagocytosis was then evaluated using CelllnsightCX5 High Content Screening Platform and analyzed using HCS Studio Cell Analysis Software. Phagocytosis was quantified using image-based analysis as the average number of pHrodo-bright tumor cells for 100 cell trace violet-positive macrophages (Phagocytosis Index). Relative Phagocytosis index was calculated as following: Phagocytosis Index (treatment)-Phagocytosis Index (untreated condition).

Statistics: Ordinary One-Way Anova with Multiple comparisons, Tukey's corrections. *p<0.05; **p<0.01, ***p<0.001.

### Results and Conclusions

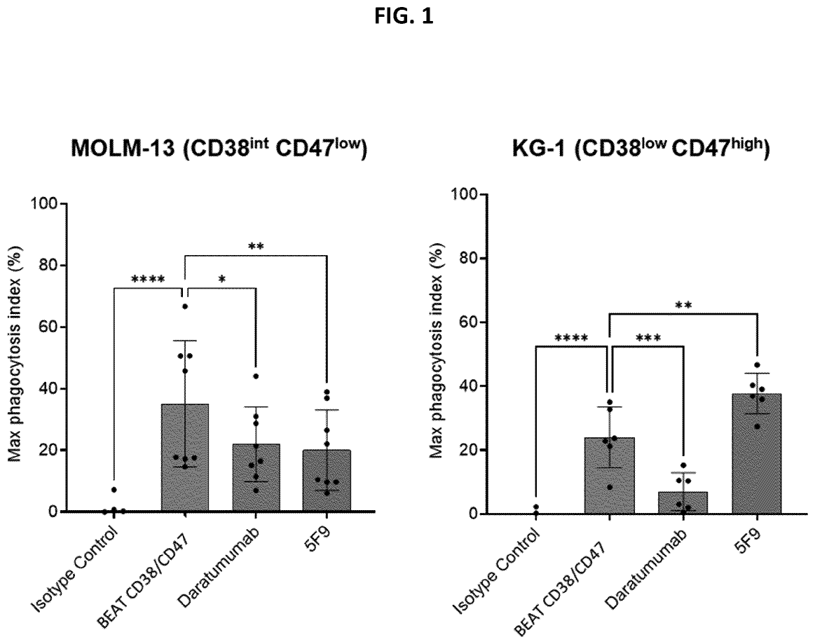

We tested the capacity of BEAT CD38/47 to induce phagocytosis of AML tumor cell lines having different expression of CD38 and CD47 (FIG. 1). We found that BEAT CD38/47 could kill AML cell lines through antibody dependent cell phagocytosis (ADCP) as compared to isotype control. In addition, we compared the phagocytic activity of BEAT CD38/47 with that of mono-specific anti-CD38 or anti-CD47 antibodies, daratumumab or anti-CD47 (5F9) respectively. We found that BEAT CD38/47 was inducing higher ADCP of AML cell lines as compared to daratumumab and anti-CD47 (5F9) on AML cell line expressing intermediate or low level of CD38 and CD47.

These data suggest that BEAT CD38/47 is capable to induce phagocytosis of AML cell line of other origin than multiple myeloma.

### Example 2: BEAT CD38/47 Shows Higher Potency than Daratumumab in ADCC and Induces Higher Killing of AML Cell Line in MMoAK Assays as Compared to Anti-CD47 (5F9)

### Material and Methods

The potential of BEAT CD38/47 to induce ADCC of AML cell lines has been measured by quantifying the number of live tumor cells using a flow-cytometry read-out, with NK cells as effector cells and eF670-labelled tumor cells as target cells, in a 5/1 E/T ratio.

### NK Isolation

The day before the experiment, Peripheral Blood Mononuclear Cells (PMBCs) from Healthy Volunteers were harvested from buffy coats obtained from the Bern Transfusion Center. Buffy coats were diluted 1/2 in sterile PBS and then transferred in 3 SepMate tubes previously loaded with 12 ml Ficoll. Isolation was performed following manufacturer's instructions (centrifugation at 1200 g for 10 minutes with brake). The mononuclear layer was transferred into 50 ml falcon tubes, the PBMCs were washed 3 times in PBS (300 g for 10 min), counted and finally suspended at a concentration of 50×106 cells/ml in EasySep buffer for subsequent NK isolation. NK cells were purified using EasySep NK cell enrichment kit according to the manufacturer's instructions. Purified NK cells were then resuspended at 1×106/ml in complete RPMI medium and incubated overnight at 37 °C.

### ADCC

The day of the experiment, tumor cells were washed in PBS and then labelled with 1 μM eFluor670. 1×104 tumor cells were plated in a 96-well plate with increasing concentrations of BEAT CD38/47 or control antibodies. 5×104 purified NK cells were added to 1×104 eFluor670 labelled tumor cells/well to make a final Effector:Target (E:T) ratio of 5:1. The plates were incubated at 37 °C, 5% C02. After 4h30 incubation, the plates were centrifugated for 5 min at 350 g. Cells were then resuspended in 100 μl FACS buffer+azide containing either Sytox (1/2000) or Dapi (1/50000). Flow-cytometric analysis was then performed with a Cytoflex (Beckman Coulter). Viable tumor cells were identified as positive for eFluor670 and negative for Sytox dead cell stain. Absolute number of live tumor cells were then reported and % of Killing and % Specific ADCC were calculated with the following formulas: % Killing=100*(1−(Abs count/well of sample/Average of Abs count/well of no Ab)).

### MMoAK

Monocyte isolation and Monocyte-Derived-Macrophages Differentiation (MDM) 7 days before the day of the experiment, human monocytes were isolated from frozen PBMC using EasySep Monocytes Isolation kit according to the manufacturer's instructions. Isolated monocytes were differentiated into Monocyte-Derived-Macrophages by culturing them in complete medium containing 50 ng/mL human M-CSF for 7 days at a density of 0.5×106 cells/ml.

### PBMC Isolation

One day before the day of the experiment, autologous human PBMC were thawed and cultured at 1.106 cells/ml in complete RPMI-10% FBS and incubated overnight at the incubator.

### Killing Assay

On the day of the experiment, 5×104 PBMCs+5×103 autologous Monocyte-Derived-Macrophages were plated with 1×104 tumor cells labelled with 1 μM eFluor 670 in presence of 50% human serum in ultra-low attachment 96-well plates. Increasing concentration of test antibody and control antibodies was then added to the well and incubated for further 48 h at 37 °C, 5% C02. After completion of the assay, cells were centrifuged at 350 g for 5 minutes and resuspended in 100 μl FACS buffer+azide containing either Sytox (1/2000) or Dapi (1/50000). Samples were acquired using Cytoflex flow cytometer and analyzed using Flowjo software (tree star). Viable tumor cells were identified as positive for eFluor-670 and negative for Sytox dead cell stain. Absolute number of live tumor cells were then reported and % Killing was calculated with the following formula: % Killing=100*(1−(Abs.count/well of sample/Average of Abs.count/well of Target only)).

### Results and Conclusions

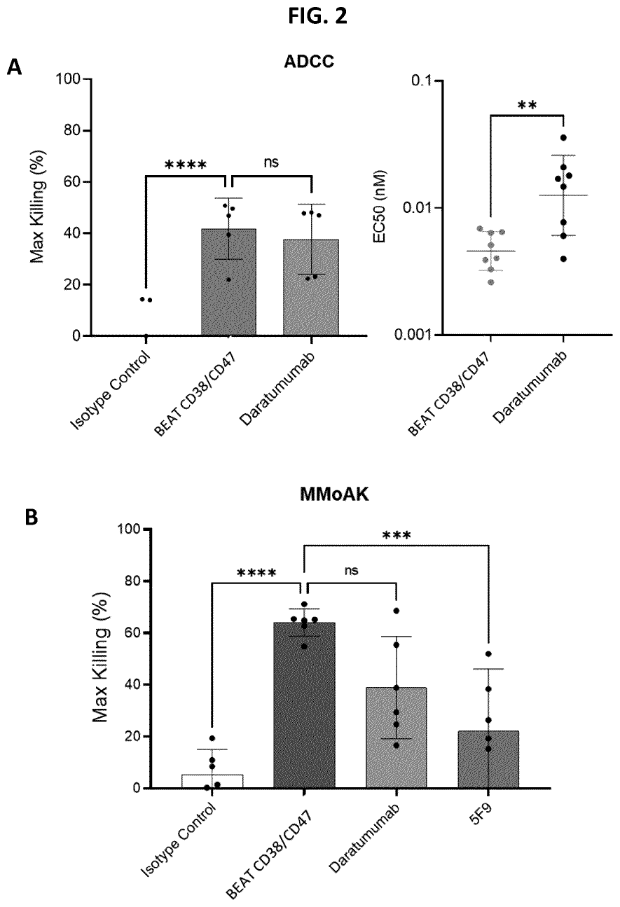

The Fc portion of BEAT CD38/47 is engineered to enhance antibody dependent cell cytotoxicity (ADCC) and complement dependent cytotoxicity (CDC). For this reason, we tested the potency of BEAT CD38/47 in vitro to induce ADCC of AML tumor cell lines (FIG. 2A). We found that BEAT CD38/47 was able to kill AML cell lines through ADCC and when compared to mono-specific anti-CD38 daratumumab, BEAT CD38/47 showed a higher potency as measured by a lower EC50 of killing.

Due to the low expression of CD38 and the high expression of complement regulatory proteins in AML cell lines, we could not detect any CDC activity induce by BEAT CD38/47 or by daratumumab (data not shown).

To characterize the complex mechanisms of action of BEAT CD38/47 in a single assay, a multiple mode of action of killing (MMoAK) in vitro assay was established where autologous macrophages and PBMCs are incubated with AML or T-ALL tumor cells and human serum. With this approach, tumor cells can be targeted simultaneously by NK cells from PBMCs (ADCC Mechanism of Action (MOA)), macrophages (ADCP MOA), and complement from human serum (CDC MOA). BEAT CD38/47 induced higher killing of AML and T-ALL cell lines in MMoAK assays as compared to daratumumab or anti-CD47 (5F9) (FIG. 2B).

Altogether these data suggest that BEAT CD38/47 is capable to kill AML cell lines via multiple mode of action of killing dependent on its enhanced Fc functionality.

### Example 3: BEAT CD38/47 Induces Higher Killing of T-ALL Cell Lines with Intermediate or Low CD38 Expression as Compared to Daratumumab as Measured by ADCC or Phagocytosis

### Material and Methods

Material and methods are the same as described in Examples 1 and 2.

### Results and Conclusions

T-ALL is characterized with high CD38 expression and daratumumab in combination with chemotherapy is under investigation (NCT03384654) for this indication. In addition, co-targeting of CD38 and CD47 by combination of antibodies has recently demonstrated efficacy in multiple preclinical models of refractory T-ALL.

Based on the above, BEAT CD38/47 has the potential to treat T-ALL patients with its first-in-class molecular attributes of co-targeting CD38 and CD47 by a single antibody with enhanced Fc effector functions.

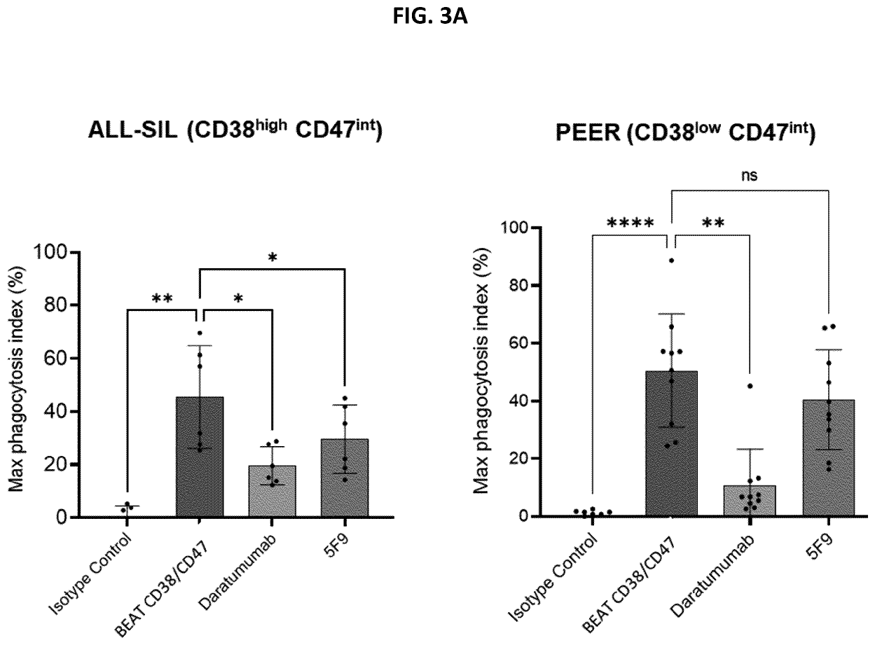

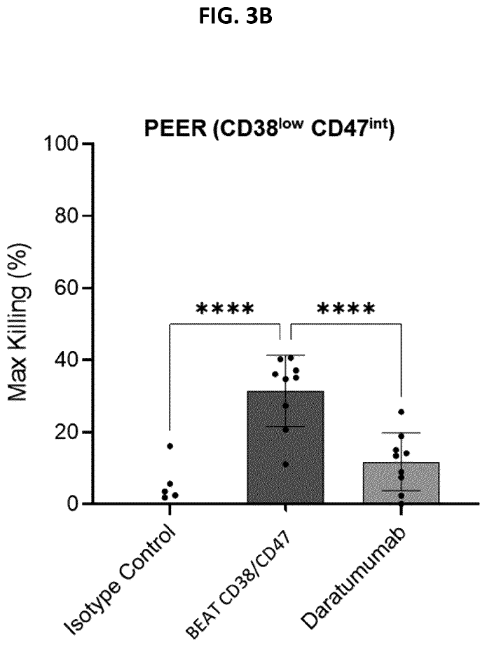

We tested the capacity of BEAT CD38/47 to induce phagocytosis of and ADCC of T-ALL tumor cell lines having different expression of CD38 and CD47. We found that BEAT CD38/47 could kill AML cell lines through antibody dependent cell phagocytosis (ADCP) as compared to isotype control. In addition, we compared the phagocytic activity of BEAT CD38/47 with that of mono-specific anti-CD38 or anti-CD47 antibodies, daratumumab or anti-CD47 (5F9) respectively. We found that BEAT CD38/47 was inducing higher ADCP of T-ALL cell lines as compared to daratumumab on cell line expressing intermediate or low level of CD38 and CD47 (FIG. 3A).

We also determined the ADCC activity against T-ALL cell lines and found that BEAT CD38/47 induces higher killing of tumor cells mediated by NK cells as compared to that induced by daratumumab (FIG. 3B). Altogether these data suggest that BEAT CD38/47 is capable to induce killing of T-ALL cell lines with multiple mode of action, including phagocytosis and ADCC supporting the development of BEAT CD38/47 in this tumor indication.

### Example 4: BEAT CD38/47 Induces Higher Killing of T-ALL Cell Lines with Intermediate or Low CD38 Expression as Compared to Daratumumab as Measured by ADCC or Phagocytosis

### Material and Methods

A killing assay with frozen BMMC was set-up to assess BEAT CD38/47 potency on AML patient sample. Briefly, BMMN were thawed in 5 ml IMDM containing 20% FBS and 0.1 mg/ml Dnasel/Stemcell technology). Cells were incubated at room temperature until use in subsequent assays.

BMMC were then counted, centrifugated and resuspended at 1×106/ml in StemSpan II (StemCell) containing CC100 (2× concentrated, from StemCell) and 10% human serum (from Bern Transfusion Center). 100 ul of cell suspension was distributed in each well of a U-bottom 96w plate. Antibodies were prepared at 2× in StemSpam II medium from 100 nM, diluted 1/10 on 8 dilutions down. 100 ul/well antibody were distributed on top of cells. Cells were incubated with antibodies for 20-24 h at 37 °C, 5% CO2. After 20-24 h culture at 37 °C, cells were centrifugated and stained with 50 ul/well of FACS buffer containing a mix of antibodies (CD117-BV650, CD45-APC-Fire 750, CD19-AF405 and CD34-pecya7). After 15-20 min incubation with antibodies, cells were washed 2 times and finally resuspended in 200 ul/well FACS Buffer containing Dapi (diluted 1/50000). Remaining live CD34+CD45low blast cells were quantified in each well. % Killing vs untreated condition was determined by the formula: % Killing=100*(1-absolute number cells treated condition/absolute number cells untreated condition).

### Results and Conclusions

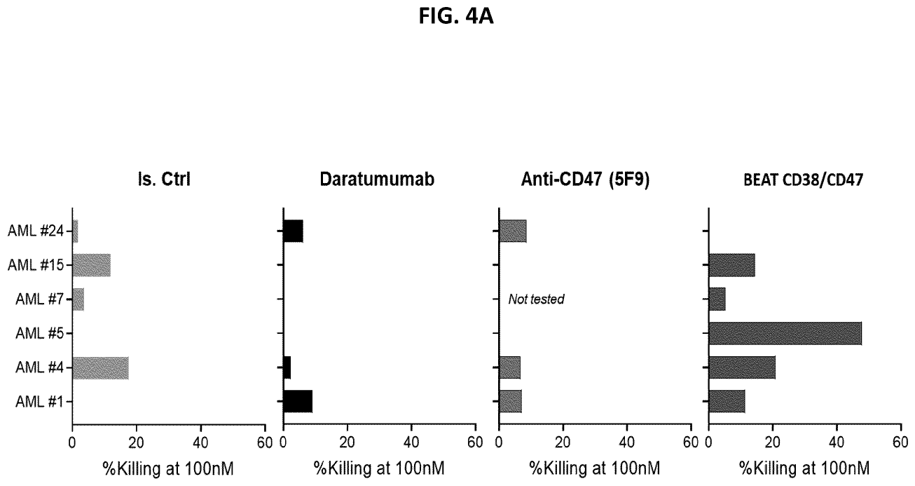

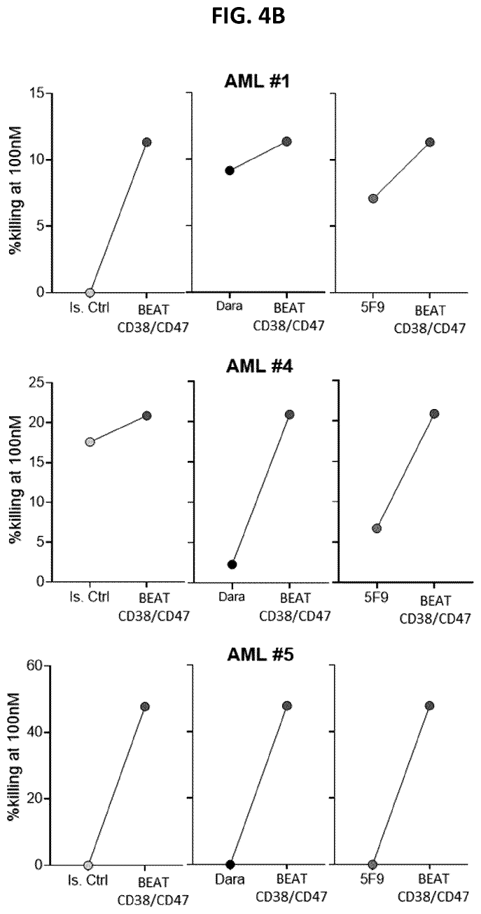

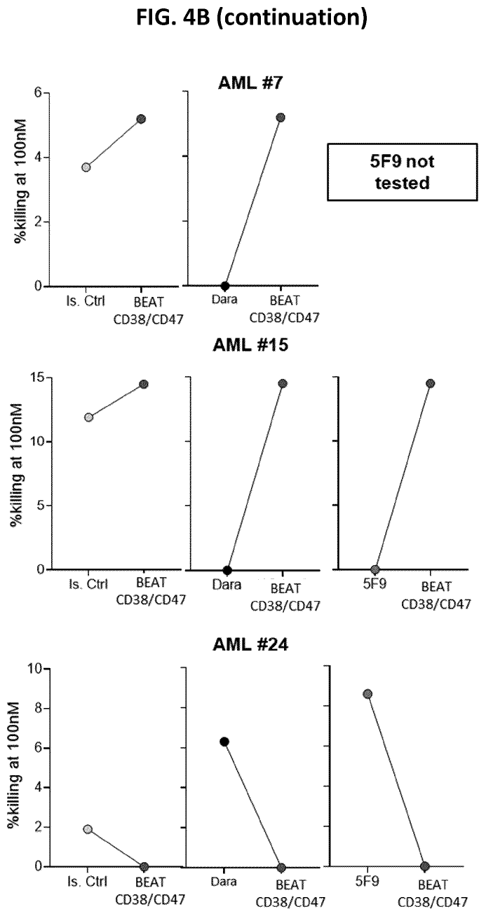

To get a better clinical relevance of the BEAT CD38/47 potency for AML indication, we tested the activity of BEAT CD38/47 ex vivo using bone marrow aspirates of AML patients. BEAT CD38/47 was tested in a direct killing assay head-to-head to mono-specific anti-CD38 or anti-CD47 monoclonal antibodies, using daratumumab or anti-CD47 (5F9) respectively. In such an assay, tumor cells a depleted by antibodies based on the tumor contextures present in the aspirate. Most of the samples showed low or absent effector cells and were primarily composed by AML blasts. In addition, multiple immune suppressive mechanisms hamper the activity of direct killing in such a direct assay ex vivo. For these reasons, we detected activities of BEAT CD38/47 or control antibodies only in 5 out of 12 patients samples tested (data not shown). Among the samples where AML blast killing was detectable, BEAT CD38/47 induced killing in 5 out of 6 samples tested, as compared to daratumumab or anti-CD47 (5F9) that induced killing only in 3 out of 6 samples (FIG. 4A).

In addition, when looking at each single patient samples, BEAT CD38/47 induced a trend of a higher killing of AML blast as compared to daratumumab and anti-CD47 (5F9) in 5 out of 6 samples (FIG. 4B).

Altogether these data suggest that BEAT CD38/47 can induce killing of AML blast in a translational relevant direct ex vivo killing approach.

### Example 5: BEAT CD38/47 Induced Killing of Primary AML Cells Ex Vivo

### Materials and Methods

Human frozen AML samples were obtained from MDACC. Briefly, BMMN were thawed in IMDM containing 20% FBS and 0.1 mg/ml Dnasel (Stemcell technology). BMMC were then centrifugated and resuspended in StemSpan II (StemCell) containing CC100 (StemCell) and 10% human serum (Bern Transfusion Center). Cells were then incubated with increasing dose of antibodies at 37 °C, 5% C02. After 20-24 h culture, cells were centrifugated and stained with a mix of antibodies composed of CD117-BV650 (BD), CD45-APC-Fire 750 (Biolegend) and CD34-pecya7 (BD). After 20 min incubation with antibodies, cells were washed 2 times and finally resuspended in PBS containing 2.5% FCS 2 mM EDTA and 0.05% NaN3 and a viability dye. (Dapi). Live CD34+CD45low AML blast cells were quantified for each condition. % Killing vs untreated condition was determined by the formula: % Killing=100*(1-absolute number cells treated condition/absolute number cells untreated condition).

### Results and Conclusions

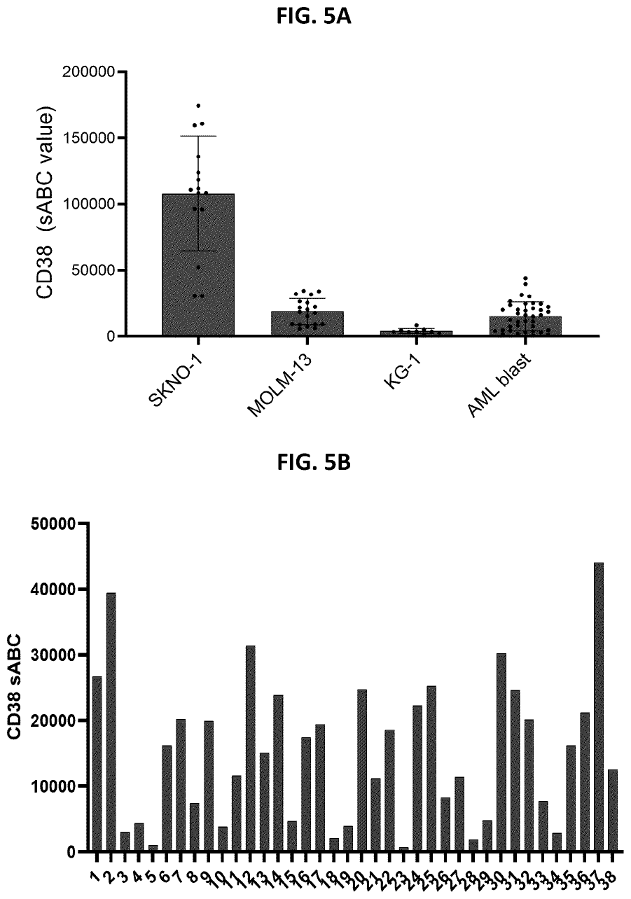

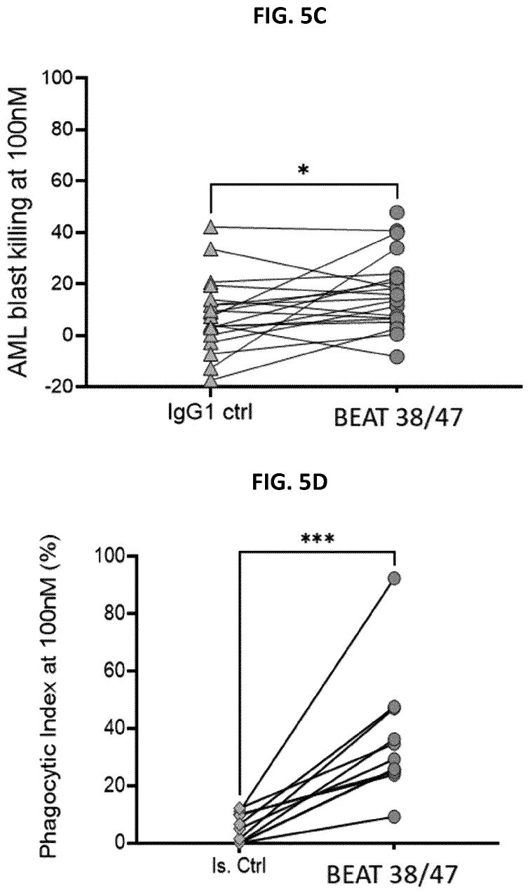

The potency of BEAT CD38/47 ex vivo using bone marrow (BM) aspirates from AML patients was investigated. CD38 expression on AML blasts was heterogeneous between different samples analyzed (FIG. 5A-B).

Similar to what was found in vitro, BEAT CD38/47 induced killing of AML blasts ex vivo compared to isotype control (FIG. 5C). Because bone marrow aspirates might not contain tissue resident macrophages, which are a major cell type driving the BEAT CD38/47 MOA, we implemented ex vivo killing assays with monocytes derived macrophages (MDM) and checked depletion of AML blasts by flow cytometry. Addition of MDM further enhanced the percentage of AML primary blast cell killing (FIG. 5D). Using the same approach, two primary MM samples were also tested ex vivo and showed a similar trend of enhanced killing of CD138+MM cells when MDM were added as effector cells (data not reported). This suggests that in AML primary samples, absence of tissue resident macrophages could hamper BEAT CD38/47 activities detected ex vivo.

1. A bispecific antibody comprising at least one binding portion which binds to human CD47 and at least two binding portions which bind to human CD38, for use in the treatment of a subject having Acute myeloid leukemia or T-cell acute lymphoblastic leukemia.

2. The bispecific antibody for use according to claim 1, wherein said at least two CD38 binding portions are biparatopic.

3. The bispecific antibody for use according to any one of claim 1 or 2, wherein said at least one binding portion which binds to human CD38 comprises a CDR set selected from the group comprising: SEQ ID NO: 103, SEQ ID NO: 163, SEQ ID NO: 223; SEQ ID NO: 117, SEQ ID NO: 177, SEQ ID NO: 237; SEQ ID NO: 121; SEQ ID NO: 181, SEQ ID NO: 241.

4. The bispecific antibody for use according to any one of the preceding claims, wherein said at least one binding portion which binds to human CD38 comprises a CDR set comprising the amino acid sequence of SEQ ID NO: 117, SEQ ID NO: 177, SEQ ID NO: 237; and a CDR set comprising the amino acid sequence of SEQ ID NO: 121; SEQ ID NO: 181, SEQ ID NO: 241.

5. The bispecific antibody for use according to any one of the preceding claims, wherein said at least one binding portion which binds to human CD47 comprises a CDR set comprising the amino acid sequence of SEQ ID NO: 75, SEQ ID NO: 135, SEQ ID NO: 195.

6. The bispecific antibody for use according to any one of the preceding claims, further comprising a common light chain.

7. The bispecific antibody for use according to any one of the preceding claims, wherein said common light chain comprises an amino acid sequence of SEQ ID NO: 10.

8. The bispecific antibody for use according to any one of the preceding claims, wherein said bispecific antibody comprises a Fc region.

9. The bispecific antibody for use according to claim 8, wherein said Fc region is a variant which comprises at least one amino acid modification relative to the Fc region of the parent antibody, whereas the antibody comprising the variant Fc region exhibits altered effector function compared to the parent antibody.

10. The bispecific antibody for use of any one of the preceding claims, wherein said at least one binding portion which binds to human CD47 has an affinity to human CD47 lower than the affinity that said at least one binding portion which binds to human CD38 has to human CD38.

11. A pharmaceutical formulation comprising the bispecific antibody of any one of the claims 1 to 10 and a pharmaceutically acceptable carrier.
